(19) United States
(12) Patent Application Publication
(10) Pub. No.: US 2006/0246438 A1
(43) Pub. Date: Nov. 2, 2006

(54) Methods for the chemical and physical modification of nanotubes, methods for linking the nanotubes, methods for the directed positioning of nanotubes, and uses thereof

(76) Inventors: Maxine McCall, New South Wales (AU); Minoo Moghaddam, New South Wales (AU)

Correspondence Address: MCDERMOTT WILL & EMERY LLP, 600 13TH STREET, N.W., WASHINGTON, DC, 20005-3096, US

(21) Appl. No.: 10/526,050

(22) PCT Filed: Aug. 29, 2003
(86) PCT No.: PCT/AU03/01118

§ 371 Date: Feb. 6, 2006

(30) Foreign Application Priority Data
Aug. 30, 2002 (AU) 2002951274

(51) Int. Cl. C12Q 1/68; C07H 21/04

(52) U.S. Cl. 435/6; 977/924; 536/24.3

(57) ABSTRACT

The invention relates to methods for chemically and physically modifying nanotubes with nucleic acid, and uses thereof. In particular, the invention relates to one or more nanotube(s) with one or more numcleic acid molecule(s) with one or more nucleic acid molecule(s) attached thereto.

### FIELD OF THE INVENTION

The invention relates to methods for chemically and physically modifying nanotubes with nucleic acid, and uses thereof. The invention also relates to linked nanotubes, in particular methods for controlling the linking of such nanotubes. The invention also relates to devices and applications which require the placement of nanotubes in specific locations, in particular methods for controlling the directed positioning of such nanotubes. The invention also relates to the DNA patterning on nanotubes and a method for placing multiple layers of nanoparticles on the surface of nanotubes.

### BACKGROUND OF THE INVENTION

Nanotubes are typically small cylinders made of organic or inorganic materials. For example, known types of nanotubes include peptidyl nanotubes and carbon nanotubes.

Carbon nanotubes are cylindrical shells of graphitic sheets typically having diameters of 1-300 nanometers and lengths of 1-100 μm and sometimes up to mm in size. They offer unique physical properties that are potentially useful in a variety of nanometer-scale devices and technologies. Indeed, carbon nanotubes have been proposed as new materials for electron field emitters in panel displays, single-molecular transistors, scanning probe microscope tips, gas and electrochemical energy storage, catalyst and protein/DNA supports, molecular-filtration membranes, and energy-absorbing materials (see, for example, Dekker, “Carbon nanotubes as molecular quantum wires,” Physics Today, May 1999, M. Dresselhaus, et al., Phys. World, Jan. 33, 1998; P. M. Ajayan, and T. W. Ebbesen, Rep. Prog. Phys., 60, 1027, 1997; R. Dagani, CE News, Jan. 11, 31, 1999).

However, most of the envisioned applications require that the nanotubes are grown in a highly controlled fashion, i.e., with their diameter, length, location and microstructure, controllable and reproducible. Unfortunately, carbon nanotubes synthesised by most of the common techniques, such as arc discharge and catalytic pyrolysis, often exist in a randomly entangled state (see, for example, T. W. Ebbesen and P. M. Ajayan, Nature 358, 220, 1992; M. Endo et al., J. Phys. Chem. Solids, 54, 1841, 1994; V. Ivanov et al., Chem. Phys. Lett., 223, 329, 1994). In recent times, these problems have largely been overcome by preparing aligned carbon nanotubes either by post-synthesis manipulation (see, for example, M. Endo, et al., J. Phys. Chem. solids, 54, 1841, 1994; V. Ivanov, et al., Chem. Phys. Lett., 223, 329, 1994; H Takikawa, et al., Jpn. J. Appl. Phys., 37, L187, 1998), or by synthesis-induced alignment (see, for example, W. Z. Li, Science, 274, 1701, 1996; Che. G., Nature, 393, 346, 1998; Z. G. Ren, et al., Science, 282, 1105, 1998; C. N., Rao, et al., J. C. S., Chem. Commun., 1525, 1998).

Aligned nanotubes have also been synthesised using porous templates (W. Z. Li et al., “Large Scale Synthesis of Aligned Carbon Nanotubes,” Science, Vol. 274, 1701 (1996); S. Fan et al., “Self-oriented regular arrays of carbon nanotubes and their field emission properties,” Science, Vol. 283, 512 (1999); J. Li et al., “Highly ordered carbon nanotubes arrays for electronic applications”, Appl. Phys. Lett., Vol. 75, 367 (1999)). Other papers on growing aligned nanotubes have described DC plasma assisted hot filament deposition (Z. F. Ren et al., “Synthesis of large arrays of well-aligned carbon nanotubes on glass,” Science, Vol. 282, page 1105 (1998)).

Despite all of the developments in the growth of nanotubes, some of the more useful applications for this technology remain elusive, as they require not only regular growth of nanotubes, but also the linking of the nanotubes either side-to-side or end-to-end.

### SUMMARY OF THE INVENTION

The inventors have now developed a process capable of linking nanotubes. Importantly, the inventors have developed a process, which allows linkage of nanotubes either side-to-side or end-to-end, thereby dramatically increasing their usefulness. The inventors have also developed a process of physically modifying the walls of nanotubes, while preserving the sp² structure of the nanotubes and thus their electronic characteristics. The inventors have also developed a method for locating nanotubes to specific targets. The inventors have also developed techniques which allow DNA patterning on nanotubes as well as the creation of multiple layers of nanoparticles on the surface of nanotubes.

In its broadest aspect, the invention provides a method of chemically attaching nucleic acid molecules to one or more nanotubes. The invention also provides a method of physically attaching nucleic acid molecules to one or more nanotubes. The invention also provides a method of linking these nanotubes. Further, the invention provides a process whereby nanotubes may be directed to specific locations.

Accordingly, in a first aspect, the present invention provides a nanotube with one or more nucleic acid molecule(s) attached thereto.

In a second aspect, the invention provides a method of chemically modifying a nanotube comprising the steps of:

(a) chemically attaching at least one linker attached to one or more nucleic acid molecules to an optionally functionalised nanotube, wherein said linker consists wholly or partly of a functional group with the proviso that when the nanotube is functionalised with CO₂H, then the linker is not a primary aliphatic alkyl amine; or

(b) chemically attaching at least one linker attached to one or more nucleic acid molecule to an optionally functionalised nanotube, wherein said linker consists wholly or partly of a functional group; and

(c) synthesising at least two nucleic acid molecules, by sequential addition of nucleotides in situ, starting from said one or more nucleic acid molecules; or

(d) chemically attaching at least one linker to an optionally functionalised nanotube, wherein said linker consists wholly or partly of a functional group; and

(e) attaching one or more nucleic acid molecules to said optionally functionalised nanotube via said functional group on said linker; or

(f) synthesising one or more nucleic acid molecules, by sequential addition of nucleotides in situ, starting from said functional group on said linker.

In a third aspect, the invention provides a method of chemically modifying a nanotube comprising the steps of:

a) photochemically attaching at least one linker attached to one or more nucleic acid molecules to an optionally functionalised nanotube, wherein said linker consists wholly or partly of a functional group; or

b) photochemically attaching at least one linker attached to one or more nucleic acid molecules to an optionally functionalised nanotube, wherein said linker consists wholly or partly of a functional group; and

c) synthesising at least two nucleic acid molecules by sequential addition of nucleotides in situ, starting from said one or more nucleic acid molecules; or

d) photochemically attaching at least one linker to an optionally functionalised nanotube, wherein the linker consists wholly or partly of a functional group; and

e) attaching one or more nucleic acid molecules to said optionally functionalised nanotube via said functional group on said linker; or

f) synthesising one or more nucleic acid molecules, by sequential addition of nucleotides in situ, starting from said functional group on said linker.

In a fourth aspect, the invention provides a method of physically modifying a nanotube comprising the steps of:

a) physically adsorbing at least one anchor attached to one or more nucleic acid molecules to the surface of an optionally functionalised nanotube, wherein said anchor consists wholly or partly of a functional group; or

b) physically adsorbing at least one anchor attached to one or more nucleic acid molecules to the surface of an optionally functionalised nanotube, wherein said anchor consists wholly or partly of a functional group; and

c) synthesising at least two nucleic acid molecules by sequential addition of nucleotides in situ, starting from said functional group on said anchor; or

d) physically adsorbing at least one anchor to the surface of an optionally functionalised nanotube, wherein said anchor consists wholly or partly of a functional group; and

e) chemically attaching one or more nucleic acid molecules to said functional group on said anchor adsorbed on the optionally functionalised nanotube; or

f) synthesising one or more nucleic acid molecules, by sequential addition of nucleotides in situ, starting from said functional group on said anchor.

In a fifth aspect, the invention provides a plurality of linked nanotubes.

In a sixth aspect, the present invention provides a method of linking nanotubes comprising the steps of:

attaching a first nucleic acid molecule of a first base sequence to a first optionally functionalised nanotube; and

hybridising the first nucleic acid molecule with a second nucleic acid molecule of a second base sequence attached on a second optionally functionalised nanotube, wherein the base sequence of the second nucleic acid molecule is substantially complementary to the base sequence of the first nucleic acid molecule.

In a seventh aspect, the present invention provides a method of linking nanotubes comprising the steps of:

a) attaching a first nucleic acid molecule of a first base sequence to optionally functionalised nanotubes; and

b) hybridising the first nucleic acid molecule with a second nucleic acid molecule which comprises a base sequence substantially complementary to the first base sequence and further comprises a second or a third base sequence which is/are not complementary to the first base sequence, but is/are complementary to each other.

In an eighth aspect, the present invention provides a method of linking nanotubes comprising the steps of:

a) attaching a first nucleic acid molecule of a first base sequence to a first optionally functionalised nanotube;

b) attaching a second nucleic acid molecule of a second base sequence to a second optionally functionalised nanotube;

c) hybridising the first nucleic acid molecule to a third nucleic acid molecule which comprises a base sequence substantially complementary to the base sequence of the first nucleic acid molecule and which further comprises at least 5 nucleotides which are not complementary to the base sequence of the first or second nucleic acid molecules;

d) hybridising the second nucleic acid molecule to a fourth nucleic acid molecule which comprises a base sequence substantially complementary to the base sequence of the second nucleic acid molecule and which further comprises at least 5 nucleotides which are not complementary to the base sequences of the first or second nucleic acid molecules;

wherein the base sequences of the third and fourth nucleic acid molecules are substantially complementary such that under stringent hybridisation conditions said third and fourth nucleic acid molecules hybridise thereby linking said first and second optionally functionalised nanotubes.

In a ninth aspect, the present invention further provides linked nanotubes produced by the method of the sixth, seventh and eighth aspects linked together to form a string of linked nanotubes. The method of linking being the same method as that disclosed in the sixth, seventh and eighth aspects, respectively, but subsequently repeated.

In a tenth aspect, the present invention provides a method of linking nanotubes comprising the steps of:

a) providing a plurality of optionally functionalised nanotubes with attached nucleic acid molecules, wherein said nucleic acid molecules have the same or different base sequences;

b) exposing said optionally functionalised nanotubes to a nucleotide strand which comprises a base sequence substantially complementary to one or more of the base sequences of said nucleic acid molecules; and

c) incubating said optionally functionalised nanotubes and nucleotide strand under appropriate hybridisation conditions wherein said optionally functionalised nanotubes are linked via hybridisation of the nucleic acid molecules with the nucleotide strand.

In an eleventh aspect, the present invention provides a method for directing nanotubes to specific targets comprising the steps of:

a) attaching a first nucleic acid molecule of a first base sequence to optionally functionalised nanotubes;

b) attaching a second nucleic acid molecule of a second base sequence which is substantially complementary to the first base sequence to a target; and

c) hybridising said first and second nucleic acid molecules.

In a twelfth aspect, the present invention provides a method for directing nanotubes to specific targets comprising the steps of:

a) attaching a first nucleic acid molecule of a first base sequence to an optionally functionalised nanotube;

b) attaching a second nucleic acid molecule of a second base sequence to a target;

c) exposing said nanotube and target to a third nucleic acid molecule which comprises a base sequence which is substantially complementary to both the first and second nucleic acid molecules; and

d) incubating said optionally functionalised nanotube and target under appropriate hybridisation conditions wherein said optionally functionalised nanotube and target are linked via hybridisation of the first and second nucleic acid molecule via the third nucleic acid molecule.

In a thirteenth aspect, the present invention provides a method for directing nanotubes to specific targets comprising the steps of:

a) attaching a first nucleic acid molecule of a first base sequence to an optionally functionalised nanotube;

b) attaching a second nucleic acid molecule of a second base sequence to a target;

c) hybridising the first nucleic acid molecule to a third nucleic acid molecule which comprises a base sequence substantially complementary to the base sequence of the first nucleic acid molecule and which further comprises at least 5 nucleotides which are not complementary to the base sequence of the first or second nucleic acid molecules;

d) hybridising the second nucleic acid molecule to a fourth nucleic acid molecule which comprises a base sequence substantially complementary to the base sequence of the second nucleic acid molecule and which further comprises at least 5 nucleotides which are not complementary to the base sequences of the first or second nucleic acid molecules;

wherein the base sequences of the third and fourth nucleic acid molecules are substantially complementary such that under stringent hybridisation conditions said third and fourth nucleic acid molecules hybridise thereby directing said optionally functionalised nanotube to said target.

In a fourteenth aspect, the present invention provides a method for directing nanotubes to specific targets comprising the steps of:

a) attaching a first nucleic acid molecule of a first base sequence to optionally functionalised nanotubes;

b) attaching a second nucleic acid molecule of a second base sequence to a target, where the second base sequence is not complementary to the first base sequence, and where the second base sequence may or may not be the same as the first base sequence, and

c) adding a third nucleic acid molecule which has in one part a base sequence substantially complementary to the base sequence of the first nucleic acid molecule and in another part a base sequence substantially complementary to the base sequence of the second nucleic acid molecule; and

d) hybridising the third nucleic acid molecule to the first and the second nucleic acid molecules, thus linking the optionally functionalised nanotube to the target.

In a fifteenth aspect, the invention provides a nucleic acid sensor comprising a nanotube with one or more nucleic acid molecule(s) attached thereto, wherein the base sequence of the said attached nucleic acid molecule is substantially complementary to all or a portion of the base sequence of the nucleic acid molecules being detected.

In one embodiment, the sensor consists of an array of groups of one or more nanotubes, each group having one or more nucleic acid molecules of the same base sequence attached to each nanotube in the group, and where the base sequence of the nucleic acid molecules attached to the nanotubes in one group differs from those in other groups so that a number of different target DNA molecules may be detected.

In a sixteenth aspect, the invention provides an actuator comprising one or more nanotubes with one or more nucleic acid molecule(s) attached thereto and a membrane support to which the DNA-modified nanotubes are attached.

In a seventeenth aspect there is provided a conductor or semi-conductor comprising one or more nanotubes with one or more nucleic acid molecule(s) attached thereto.

The conductor may be a metallic conductor.

In one embodiment, the conductor is a nanowire comprised of nanotubes linked together via nucleic acid hybridisation. The nanowire may further comprise nanoparticles or coating of conductive material.

In one embodiment, one or more nanotubes comprising one or more nucleic acid molecule(s) attached thereto are exposed to nanoparticles comprising a plurality of complementary nucleic acid molecules attached thereto, wherein said nanoparticles hybridize to the nucleic acid molecules on the surface of the nanotube(s) as well as self-anneal to other nanoparticles thereby forming one or more coated nanotubes.

The nanotubes may be made from any suitable material already known in the art. The nanotubes may be carbon nanotubes. The carbon nanotubes may be grown using any known procedure in the art; for example, Arc discharge method, chemical vaporisation deposition method (CVD), plasma enhanced chemical vaporisation deposition method (PECVD), laser ablation/vaporization, pyrolysis, thermal chemical vapour deposition, electrolysis, flame synthesis, or a combination of these techniques for the manufacture of either multi-walled nanotubes (MWNTs) or single-walled nanotubes (SWNTs). The type of nanotube used depends partly on the end use of the nanotube. For example, the nanotubes may be aligned, aligned and patterned, or dispersed nanotubes. The nanotubes may also be SWNTs or MWNTs.

Each of the first, second, third or fourth nucleic acid molecules may be DNA, cDNA, RNA, oligonucleotide, oligoribonucleotide, modified oligonucleotide, modified oligoribonucleotide, peptide nucleic acid (PNA), or hybrid molecules thereof. In one embodiment, the nucleic acid molecule is an oligonucleotide.

The nucleic acid molecule may be synthesised in a DNA synthesiser or produced by enzymatic digestion or enzymatic polymerisation and then attached on to the nanotube by any method known including by reacting the nucleic acid molecule with or without a linker to a nanotube modified with a functional group or with a nanotube physically modified with an anchor containing a functional group. Alternatively, the nucleic acid molecule may be synthesised in situ onto a functionalised nanotube or onto a nanotube physically modified with an anchor containing a functional group.

In one embodiment, the synthesised nucleic acid molecule is attached to a nanotube modified with carboxyl groups either by oxidation or by photo-irradiation of an azido linker containing carboxyl groups. The carboxyl group on the nanotube or on the azido linker forms an amide bond with 5′ or 3′amino modified DNA. This amide bond might be extended by incorporating a spacer between the DNA and the linker by using difunctional reagents such as standard amino acids or non-standard amino acids (C3-C12), for example, 11-amino undecanoic acid and peptides.

In another embodiment the nucleic acid molecule is synthesised in situ by either oxidizing nanotubes to form hydroxyl groups or attaching functional hydroxyl groups to the nanotubes using photochemical reaction of azido compounds such as azido thymidine or azidoadenosine via the azide functional group to the nanotube. The DNA molecule is built up by sequentially adding nucleotides by phosphoramidite chemistry used in automated DNA synthesis.

In yet a further embodiment, the DNA is physically attached to the nanotube via a covalent linkage to an anchor which is physically adsorbed to the surface of the nanotube. The DNA may be pre-synthesised or synthesised in situ. The anchor typically contains a hydrophobic domain such as a pyrenyl, porphyrin or acridine derivative which interact strongly with the hydrophobic walls of the nanotube, and a functional group to which the DNA can be attached or built-up from. Alternatively the anchor may be an oligonucleotide spacer such as oligo thymidine or oligo guanidine which physically adsorbs to the nanotube walls and from which extends the hybridizing DNA.

The nucleic acid molecule may be attached to the walls(s) or side(s) and/or the tip(s) of the nanotube.

The carbon nanotubes can be linked end-to-end, side-to-side, or combinations thereof and the linking process utilises the unique self-annealing properties of nucleic acids. In one embodiment, the linking process involves the attachment of a single-stranded nucleic acid molecule to the side or end of a first nanotube, and the attachment of a complementary single-stranded nucleic acid molecule to the side or end of a second nanotube, wherein, under appropriate hybridisation conditions, the nucleic acid molecules hybridise together thereby linking the nanotubes.

In another embodiment, the linking process involves the attachment of a first single-stranded nucleic acid molecule to the side or end of carbon nanotubes. As all of the carbon nanotubes comprise the same single-stranded nucleic acid, there is no self-annealing. The nanotubes are then exposed to a second nucleic acid molecule, which comprises a segment of single-stranded nucleic acid, which is complementary to the first nucleic acid molecule. The second nucleic acid molecule also comprises a further segment of nucleic acid sequence which is either the “positive” strand or “negative” (complementary) strand of nucleic acid. Accordingly, in this embodiment, the complementary strands of the first and second nucleic acids hybridise and then the positive and negative strands of the second nucleic acid, which are complementary, hybridize thereby linking the nanotubes by a “bridge structure”. This “bridge structure” may be used to increase the distance between nanotubes without requiring the synthesis of long strands of nucleic acid, which may suffer problems of self-complementarity and the like. It will be appreciated by those skilled in the art that further variations on this embodiment may be created such as the second nucleic acid having non-complementary nucleic acid segments, but having complementary sequences with a third, fourth or more nucleic acid molecules.

In a further embodiment, the nanotubes may be linked by hybridising nanotubes comprising a first attached nucleic acid molecule to a second nucleic acid molecule, which comprises two or more complementary nucleic acid sequences as contiguous repeats or non-contiguous repeats. Under appropriate hybridisation conditions a string of nanotubes, joined via the second nucleic acid molecule, is produced.

The linked carbon nanotube may also include other nanoparticles including spheres, rods, octahedrons, which may be made of any material including transition metals, for example, gold, silver, and cadmium sulphide (CdS). The other nanoparticles may be incorporated in and/or coated on to the nanotube, nucleic acid molecule, nanotube with the attached nucleic acid molecule and/or the linked nanotube.

### BRIEF DESCRIPTION OF THE FIGURES

FIG. 1 shows Scheme 1 which illustrates the chemical attachment of DNA to nanotubes via hydroxyl groups and (a) DNA synthesis in situ or (b) attachment of pre-synthesised DNA. Here, XY is the coupling product formed by reacting X and Y functional groups.

FIG. 2 shows Scheme 2 which illustrates the chemical attachment of DNA to uncoated nanotubes via carboxyl groups and (a) attachment of pre-synthesised DNA or (b) DNA synthesis in situ.

FIG. 3 shows Scheme 3 which illustrates the photochemical modification of nanotubes via azidothymidine and (a) DNA synthesis in situ or (b) attachment of pre-synthesised DNA. Here, XY is the coupling product formed by reacting X and Y functional groups.

FIG. 4 shows Scheme 4 which illustrates the photochemical modification of nanotubes via photo-etching linkers and (a) attachment of pre-synthesised DNA or (b) DNA synthesis in situ. NHS is N-hydroxysuccinimide.

FIG. 5 shows a schematic diagram of gold nanoparticles functionalised with DNA. The gold nanoparticles are depicted by balls, and the oligonucleotides by black lines.

FIG. 6 shows a TEM image of gold nanoparticles functionalised with DNA. The DNA cannot be seen in this image.

FIG. 7 shows TEM images of multi-walled nanotubes and gold nanoparticles with and without DNA, showing hybridization of DNA-modified gold nanoparticles to DNA chemically attached to the walls of MWNTs, and controls. The DNA has been chemically attached to the MWNTs by a photochemical reaction of azidothymidine with the nanotubes, followed by in situ synthesis of DNA using phosphoramidite chemistry. The DNA (NT3′) chemically attached to the nanotubes, and its complementary DNA′ (Gold2A-SH3′) bound to the gold nanoparticles, each are 16 nucleotides long. (a) NT-DNA+AuNP-DNA′, (b) NT-DNA+AuNP, (c) NT+AuNP-DNA′ (d) NT+AuNP. NT represents MWNT, AuNP represents gold nanoparticle.

FIG. 8 shows TEM images of multi-walled nanotubes and gold nanoparticles with and without DNA, showing hybridization of DNA-modified gold nanoparticles to DNA chemically attached to the walls of MWNTs, and controls. The DNA is chemically attached to the MWNTs by a photochemical reaction of azidothymidine with the nanotubes, followed by in situ synthesis of DNA using phosphoramidite chemistry. Here, the DNA (NT3′-T₁₉) chemically attached to the carbon nanotubes is 35 nucleotides long. The DNA bound to the gold nanoparticles is either 16 (Gold2A-SH3′) or 35 (Gold3A-SH3′) nucleotides long. The 16-nucleotide NT3′ portion of NT3′-T₁₉ has a base sequence which is complementary to Gold2A-SH3′, and to 16 nucleotides of the 35-nucleotide Gold3A-SH3′. (a) NT-DNA+AuNP-Gold2A-SH3′ (sample I), (b) NT+AuNP-Gold2A-SH3′ (sample L), (c) NT-DNA+AuNP-Gold3A-SH3′ (sample J), (d) NT+AuNP-Gold3A-SH3′ (sample M), (e) NT-DNA+AuNP (sample K), (f) NT+AuNP (sample N). NT represents MWNT, AuNP represents gold nanoparticle.

FIG. 9 shows TEM images of single-walled nanotubes and gold nanoparticles with and without DNA, showing hybridization of DNA-modified gold nanoparticles to DNA chemically attached to SWNTs, most probably at their tips, plus controls. Here, pre-synthesised DNA with an amine linker on its 3′ end is chemically attached through an amide bond to the SWNTs which had been functionalised with carboxyl groups. The DNA (NT3′-NH₂3′) attached to the nanotubes is 16 nucleotides long. The DNA′ (Gold2A-SH3′) bound to the gold nanoparticles is also 16 nucleotides long and has a base sequence complementary to that of NT3′. (a) NT-DNA+AuNP-DNA′ (sample C), (b) NT-DNA+AuNP (sample D), (c) NT+AuNP-DNA′ (sample E), (d) NT+AuNP (sample F). NT represents SWNT, AuNP represents gold nanoparticle.

FIG. 10 shows TEM images showing hybridization of DNA-modified gold nanoparticles to DNA physically attached to MWNTs (sample 1) and to DNA chemically attached to MWNTs (sample 2), and controls. Here, the DNA (NT3′-T19) synthesized in situ in the presence of sample 1 (unmodified, aligned MWNT) and sample 2 (aligned MWNT modified by a photochemical reaction with azidothymidine) is 35 nucleotides long. Sample 3 (unmodified, aligned MWNT) has not been subjected to DNA synthesis. The 16-nucleotide DNA molecule (Gold2A-SH3′) attached to the gold nanoparticles has a base sequence complementary to the 16-nucleotide NT3′ portion of NT3′-T19. (a) sample 1 with gold nanoparticles modified with Gold2A-SH3′ (b) sample 1 with gold nanoparticles alone, (c) sample 2 with gold nanoparticles modified with Gold2A-SH3′, (d) sample 2 with gold nanoparticles alone, (e) sample 3 with gold nanoparticles modified with Gold2A-SH3′, (f) sample 3 with gold nanoparticles alone.

FIG. 11 shows TEM images of gold nanoparticles and bundled SWNTs indicating the attachment of DNA to SWNTs in mats using the ANB-NOS method. SWNT mats are photoetched with ANB-NOS, and then coupled to DNA with amine linkers through an amide bond. The DNA attached to the SWNT mats is the 35-nucleotide NT3′T₁₉NH₂3′ ((A) and (B)), and the 16-nucleotide NT3′NH₂3′ ((C) and (D)). There is no DNA attached to the SWNT mats in (E) and (F). Gold nanoparticles modified with DNA (the 35-nucleotide Gold3A-SH3′) binding to the SWNTs in (a)-(d) indicate that DNA is present on the surfaces of these SWNTs, and that it is functional. There are very few gold nanoparticles bound to the sample of unmodified SWNTs (compare (e) with (a) and (c)), indicating that the gold nanoparticles are directed to the surfaces of the SWNTs through specific DNA hybridization. Occasionally, some gold nanoparticles are located non-specifically on the surfaces of the unmodified SWNTs; an example of this is shown in (f). The scale bars in (a), (c) and (e) on the left of FIG. 9 are 500 nm. (b), (d) and (f) on the right of the figure show portions of (a), (c) and ((e), respectively, at higher magnification (scale bars are 100 nm).

FIG. 12 shows TEM images of gold nanoparticles and MWNTs indicating the attachment of DNA to aligned MWNTs using the ANB-NOS method. Aligned MWNTs on gold foil are photoetched with ANB-NOS, and then coupled to DNA with amine linkers through an amide bond. The DNA attached to the MWNTs in (b) is the 35-nucleotide NT3′T₁₉NH₂3′. There is no DNA attached to the MWNTs in (a). Gold nanoparticles modified with DNA (the 35-nucleotide Gold3A-SH3′) binding to the MWNTs in (b) indicate that DNA is present on the surfaces of these MWNTs, and that it is functional. While there are a few gold nanoparticles seen on the surfaces of the unmodified MWNTs in the control sample (a), the numbers are significantly less than in (b), indicating that physical adsorption of the DNA-modified gold nanoparticles on the MWNTs is not significant. The scale bars in (a) and (b) are 100 nm.

FIG. 13 shows TEM images of DNA-modified gold nanoparticles (16 nm diameter) bound to SWNTs functionalised with DNA at their tips or at defects in their sidewalls. (a) Attachment of gold nanoparticles to the tips of SWNTs shown here in bundles. Scale bar 200 nm. (b) Two gold nanoparticles at the tip of a SWNT or a small bundle of SWNTs (scale bar 50 nm). (c) A gold nanoparticle attached to a SWNT, either at a defect on the sidewall, or possibly linking the tips of two SWNTs (scale bar 50 nm). (d) A string of SWNTs possibly connected end-to-end by DNA-modified gold nanoparticles linked to the tips of two SWNTs through DNA hybridisation (scale bar 200 nm).

FIG. 14A shows an overlay of consecutive TEM images taken across a thin cross-section of a sample of DNA-modified aligned MWNTs to which DNA-modified gold nanoparticles had been added. The gold nanoparticles are close to the surfaces of the nanotubes throughout the sample indicating that attachment of DNA to MWNTs by the ANB-NOS method was successful. FIG. 14B shows an enlargement of image h in FIG. 14A.

FIG. 15 shows an SEM image of the gap separating the gold electrodes sitting on a Nb thin film.

FIG. 16 shows radiographs from a PhosphorImager for typical experiments to determine conditions for binding DNA molecules with disulphide linkers to gold surfaces for optimal hybridization with ³²P-labelled DNA molecules of complementary base sequence. Dark areas indicate the presence of ³²P-labeled DNA molecules. (A) (upper image) Finding the optimal concentration of Gold2A-SH3′ in 0.5M sodium phosphate buffer, pH 8, for binding in 2 hours to the gold surface, followed by soaking for 1 hour in 1 mM mercaptohexanol in ethanol. The ³²P labeled NT3′ was added. (A) (lower image) Testing the same conditions as in (A) (upper image) but with soaking in 10 mM mercaptohexanol in ethanol. The data show that 3 μM Gold2A-SH3′ is optimal under these conditions. (B) Finding the optimal concentration of sodium phosphate buffer for immobilizing 1 μM Gold2A-SH3′ or 1 μM Gold3A-SH3′ in 2 hours, followed by soaking in 1 mM mercaptohexanol for 2 hours. The data show that optimal binding of the shorter Gold2A-SH3′ is more sensitive to the buffer concentration than the longer Gold3A-SH3′.

FIG. 17(A) shows an STM image of DNA-modified MWNTs spin-coated on to a flat gold surface. STS measurements were made at four positions on a DNA-modified MWNT specified by the points A, B, C and D.

FIG. 17(B) shows current versus voltage (I-V) curves, A, B, C and D, measured by Scanning Tunnelling Spectroscopy at the specified locations A, B, C and D, respectively, on the DNA-modified MWNT shown in FIG. 17 (A). Each curve represents the average of 5-10 measurements.

FIG. 17(C) shows an STM image of unmodified MWNTs spin-coated on to a flat gold surface.

FIG. 17(D) shows current versus voltage (I-V) curves measured by Scanning Tunnelling Spectroscopy for an unmodified MWNT lying on the flat gold surface shown in FIG. 17(C). The I-V curve for the gold surface is included for comparison.

FIG. 18 shows a graph measuring the current-voltage curve down the length of two DNA-modified MWNTs by STS. Here the MWNT is lifted off the gold surface by the STM tip, while the other end remains in contact with the gold surface.

FIG. 19 (A-C) shows DNA-directed assembly of gold nanoparticles of different sizes on the surfaces of MWNTs. DNA attached to the surfaces of MWNTs binds to complementary DNA bound to gold nanoparticles of 16 nm diameter, forming a monolayer of gold nanoparticles on the surfaces of the nanotubes. The remaining unbound single strands of DNA on the gold nanoparticles can bind to complementary single strands of DNA bound to gold nanoparticles of 38 nm diameter, thus forming a second layer of nanoparticles over the first layer directly attached to the nanotubes. All images are of different nanotubes. (a) Full-length view of a MWNT showing coverage of the smaller and larger gold nanoparticles over its entire length. Scale bar 500 nm. (b) and (c) Views at higher magnification of different MWNTs, showing the larger gold nanoparticles binding directly to the smaller gold nanoparticles which are bound directly to the surfaces of the nanotubes. Scale bars 100 nm.

FIG. 20 shows a radiograph from a PhosphorImager comparing the extent of non-specific and specific binding of ³²P-labelled DNA molecules to mats of DNA-modified SWNTs. The SWNT mats were photoetched with ANB-NOS, which was then coupled through amide bonds to pre-made DNA molecules with amine linkers. Non-complementary ³²P-labelled DNA added to sample (a) on the left indicates a very low level of non-specific binding to the DNA-modified SWNT mats. In contrast, a high level of specific hybridization is seen in sample (b) on the right where ³²P-labelled DNA with complementary base sequence to the DNA attached to the SWNT mats was added. The area of the sample on the left is ˜8 mm², while the area of the sample on the right is ˜10 mm².

### ABBREVIATIONS USED

AZT Azidothymidine

ANB-NOS N-5-Azidonitrobenzoyloxysuccinimide

CPG Controlled Pore Glass

DMF N,N,Dimethylformamide

DCM Dichloromethane

DIEA N,N,Diisopropylethylamine

EDTA Ethylenediaminetetraacetic acid

Fmoc-Cl Fluorenylmethoxycarbonyl chloride

Fmoc Fluorenylmethoxycarbonyl

Fmoc-HDA N-Fluorenylmethoxycarbonyl-1,6-diaminohexane

NHS N-hydroxysuccinimide

PECVD Plasma Enhanced Chemical Vapour Deposition

MWNT Multi-walled nanotube

SWNT Single-walled nanotube

PNA Peptide nucleic acid

TEM Transmission Electron Microscopy

XPS X-ray Photoelectron Spectroscopy

### DETAILED DESCRIPTION OF THE INVENTION

The practice of the present invention employs, unless otherwise indicated, conventional molecular biology or chemistry techniques within the skill of the art. Such techniques are well known to the skilled worker, and are explained fully in the literature. See, e.g., Maniatis, Fritsch & Sambrook, “Molecular Cloning: A Laboratory Manual” (1982); “Immobilised Cells and Enzymes” (IRL Press, 1986); B. Perbal, “A Practical Guide to Molecular Cloning” (1984); Sambrook, et al., “Molecular Cloning: a Laboratory Manual” (1989); Ausubel, F. et al., 1989-1999, “Current Protocols in Molecular Biology” (Green Publishing, New York); P. Y. Brucce, “Organic Chemistry” (1995); and J. McMurry, “Organic Chemistry” (1988).

All references, including any patents or patent applications, cited in this specification are hereby incorporated by reference. No admission is made that any reference constitutes prior art. The discussion of the references states what their authors assert, and the applicant reserves the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

For the purposes of this specification it will be clearly understood that the word “comprising” means “including but not limited to”, and that the word “comprises” has a corresponding meaning.

According to one aspect of the invention, carbon-containing material is formed into carbon nanotubes and then one or more nucleic acid molecules are attached thereto.

Any means of growing carbon nanotubes may be used.

The carbon-containing material may be any compound or substance which includes carbon and which is capable of forming carbon nanotubes when subjected to pyrolysis in the presence of a suitable catalyst. Examples of suitable carbon-containing materials include alkanes, alkenes, alkynes or aromatic hydrocarbons and their derivatives, for example, methane, acetylene, benzene, transition metal phthalocyanines, such as Fe (II) phthalocyanine, and other organometallic compounds such as ferrocene and nickel dicyclopentadiene.

The catalyst may be any compound, element or substance suitable for catalysing the conversion of a carbon-containing material to aligned or dispersed carbon nanotubes under pyrolytic conditions. The catalyst may be a transition metal, such as Fe, Co, Al, Ni, Mn, Pd, Cr or alloys thereof in any suitable oxidation state.

The catalyst may be incorporated into the substrate or may be included in the carbon-containing material. Examples of carbon-containing materials which include a transition metal catalyst are Fe (II) phthalocyanine, Ni (II) phthalocyanine, nickel dicyclopentadiene and ferrocene. When the catalyst and carbon-containing material are included in the same material it may be necessary to provide sources of additional catalyst or additional carbon-containing material. For example, when ferrocene is used as the catalyst and a source of carbon, it is necessary to provide an additional carbon source, such as ethylene, to obtain the required nanotube growth.

The pyrolysis condition employed will depend on the type of carbon-containing material employed and the type of catalyst, as well as the length and density of the nanotubes required.

In this regard it is possible to vary the pyrolysis conditions, such as the temperature, time, pressure or flow rate through the pyrolysis reactor, to obtain nanotubes having different characteristics.

For example, performing the pyrolysis at a higher temperature may produce nanotubes having different base-end structures relative to those prepared at a lower temperature. The pyrolysis will generally be performed within a temperature range of 800° C. to 1100° C. Similarly lowering the flow rate through a flow-type pyrolysis reactor may result in a smaller packing density of the nanotubes and vice versa. A person skilled in the art would be able to select and control the conditions of pyrolysis to obtain nanotubes having the desired characteristics.

The diameter of nanotubes is controllable by selecting a particular catalyst layer thickness. For example, by varying the thickness of a cobalt layer from 2 nm to 60 nm, the nanotube diameter goes from about 30 nm to about 150 nm. The size of the catalyst islands is determined, as least in part, by the thickness of the catalyst layer, with thin layers leading to smaller diameter islands, and thicker layers leading to larger diameter islands. The range of nanotube diameters typically attainable is 10 to 300 nm. Control runs are easily performed to determine an appropriate catalyst layer thickness for a desired nanotube diameter.

The nanotube length is primarily controlled by the duration of the high frequency PECVD process, but not in a monotonically linear fashion. There are three stages of the process as it affects length—growth, stability, and etch. Specifically, length initially increases for a certain time period (about 5 minutes from the initiation of the process). This growth stage is followed by a period of substantially slowed growth—the stability stage. And then the nanotubes begin to be etched away such that the length is reduced—the etch stage. It appears that at some point during nanotube growth catalyst particles become completely encased by graphitic shells. Once the catalyst is so encased, nanotube growth slows (stability stage), and the etching character of the high frequency PECVD process begins to predominate (etch stage). It is also possible that the increasing length of the nanotubes interferes with the ability of reactive species to reach the catalyst at the bottom of the growing tube, thereby slowing the growth. Thus, for a given set of high frequency PECVD process parameters, the duration will typically be chosen to attain a desired length, without entering into the etch stage. However, it is possible to reach any of the three stages, and it is possible for certain advantages to exist in each. For example, it is possible that moving at least partially into the etch stage will provide nanotubes with open, as opposed to capped, ends, which may be desirable for some applications. Control runs are easily performed to find a suitable process duration to provide a desired length.

In one particularly preferred embodiment aligned, multi-walled carbon nanotubes are grown by pyrolysis of iron(II) phthalocyanine (FePc) under Ar/H₂ at 800-1100° C. (Li, D.-C., Dai, L., Huang, S., Mau, A. W. H. and Wang Z. L. (2000) Chem. Phys. Lett, 316, 349-355).

Commercial suppliers of SWNTs made by continuous process include Carbon Nanotechnologies Inc. (Houston, Tex.). Commercial suppliers of SWNTs made by CVD include Iljin Nanotech Co. Ltd. (Korea). Commercial suppliers of MWNTs made by CVD include Iljin Nanotech Co. Ltd. (Korea).

Having obtained the carbon nanotubes, one or more nucleic acid molecules are attached. Attachment may be via a linker resulting in a covalent coupling, or by an anchor resulting in physical attachment.

The term “linker” includes compounds or molecules which are composed wholly or partly of at least one functional group that are capable of linking the nanotube directly to the nucleic acid molecule.

Suitable linkers for chemical or photochemical attachment include photoetchable linkers, for example, azido compounds such as azido-thymidine, azido adenosine, azido-nitrobenzyloxy succinimide, azidophenyl isothiocyanate, 4-(P-Azido salicylamido) butylamine, 4-(P-Azido salicylamido) butyl-3′ (2′-pyridylthio)propionamide, 4-(P-Azido salicylamido) butyl-maleimide propionamide, and p-azidophenyl glyoxal monohydrate.

It will be appreciated that the linker and the nucleic acid molecule may be optionally interrupted by a spacer.

The term “spacer” includes compounds or molecules that extend the nucleic acid molecule away from the surface of the nanotube and allow for easier hybridization to complementary nucleic acid molecules.

Suitable spacer groups for extending the hydroxyl group either directly attached to the nanotube or on azido compounds photoetched to the nanotubes are described by formula I:
W—S—X I

wherein W is COOH, (—CO)₂O, COCl, halide, SO₃H or ClSO₂, and the like;

S is C₁₋₁₂ alkyl, aryl, aralkyl, C₃₋₁₂ cycloalkyl or polyalkylether, and the like; and

X is COOH, COCl, halo, SO₃H, ClSO₂, CHO, NCO, NCS, SH, OH, NH₂ or maleimide, and the like.

Suitable spacer groups for extending the carboxyl group either directly attached to the nanotube or on azido compounds photoetched to the nanotubes are described by formula Ia:
Wₐ—Sₐ—Xₐ Ia

wherein Wₐ is NH₂, OH, SH or halo, and the like;

Sₐ is C₁₋₁₂ alkyl, aryl, aralkyl, C₃₋₁₂ cycloalkyl or polyalkylether, and the like; and

Xₐ is COOH, COCl, halide, SO₃H, ClSO₂, CHO, NCO, NCS, SH, OH, NH₂ or maleimide, and the like.

Where used, either alone or within other terms such as “aralkyl” and “polyalkylether”, the term “alkyl” embraces linear or branched groups having one to twelve carbon atoms. Examples of such groups include methyl, ethyl, n-propyl, isopropyl, n-butyl, isobutyl, sec-butyl, tert-butyl, pentyl, iso-amyl, hexyl, decyl and dodecyl.

The term “cycloalkyl” embraces saturated carbocyclic groups having three to twelve carbon atoms. Examples of groups include cyclopropyl, cyclobutyl, cyclopentyl and cyclohexyl.

The term “halo” means halogens such as fluorine, chlorine, bromine or iodine.

The term “aryl” means a carbocyclic aromatic system containing one, two or three rings wherein such rings may be attached together in a pendent manner or may be fused. The term “aryl” embraces aromatic groups such as phenyl, naphthyl, tetrahydronaphthyl, indane and biphenyl.

The term “aralkyl” embraces aryl-substituted alkyl groups such as benzyl, diphenylmethyl, triphenylmethyl, phenylethyl, and diphenylethyl.

The term “anchor” includes compounds or molecules with a high affinity for physical adsorption to the surface of the nanotubes.

Suitable anchors for physical attachment to the nanotubes include pyrenebutanoic acid succinimide ester to which the DNA can be attached, acridine phosphoramidite from which the DNA can be built, or a fluorescein derivative from which the DNA can be built or to which the DNA can be covalently attached. Alternatively, the anchor may be an oligonucleotide spacer such as oligo thymidine or oligo guanidine which physically adsorbs to the nanotube walls and from which extends the hybridizing DNA.

The term “nucleic acid” is synonymous with DNA, RNA, and polynucleotides. A “nucleic acid molecule” or “polynucleic acid molecule” refers herein to deoxyribonucleic acid and ribonucleic acid in all their forms, i.e., single and double-stranded DNA, cDNA, mRNA, and the like, which may include modified bases. In one preferred embodiment, the nucleic acid is an oligonucleotide, oligoribonucleotide or an RNA-DNA hybrid molecule comprising nucleotides which may be substituted or modified in their sugar, base or phosphate group, or a PNA molecule.

The nucleotides may be in the form of deoxyribonucleotides, ribonucleotides, deoxyribonucleotide-ribonucleotide hybrids, or derivatives thereof as herein described. Modified bases, sugars or phosphate linkages of nucleotides, such as phosphoramidate, or phosphorothioate linkages in the sugar phosphate chain, may also provide resistance to nuclease attack. Binding affinity may also be optimized in particular circumstances, by providing nucleotides solely in the form of nucleotides, ribonucleotides, deoxyribonucleotides, or combinations thereof.

If the nucleic acid is an oligonucleotide then the respective 3′ and 5′ termini of the oligonucleotides or alternatively the 3′ and 5′ end termini, may be modified to stabilise the nucleic acid from degradation. For example, blocking groups may be added to prevent terminal nuclease attack, in particular 3′-5′ progressive exonuclease activity. By way of example, blocking groups may be selected from substituted or unsubstituted alkyl, substituted or unsubstituted phenyl, substituted or unsubstituted alkanoyl. Substituents may be selected from C₁-C₅ alkyl; halogens such as F, Cl or Br; hydroxyl; amino; C₁-C₅ alkoxy and the like. Alternatively, nucleotide analogues such as phosphorothioates, methylphosphonates or phosphoramidates or nucleoside derivatives (such as alpha-anomer of the ribose moiety) which are resistant to nuclease attack may be employed as terminal blocking groups. The blocking group may be an inverted linkage such as a 3′-3′ thymidine linkage or a 5′-5′ pyrophosphate linkage as in the guanosine cap.

Alternatively, groups that alter the susceptibility of the nucleic acid molecule to other nucleases may be inserted into the 3′ and/or 5′ end of the nucleic acid molecule. For example, 9-amino-acridine attached to the nucleic acid molecule may act as a terminal blocking group to generate resistance to nuclease attack on the nucleic acid molecule.

It will be readily appreciated that a variety of other chemical groups, e.g. spermine or spermidine could be used in a related manner.

The nucleic acid molecule of this invention may be produced by nucleic acid synthetic techniques that are known in the art, and then attached to the nanotube, or synthesised in situ. For example, DNA can be prepared by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; the triester method of Matteucci et al., 1981, J. Am. Chem. Soc. 103:3185-3191 or automated synthesis methods; and the solid support method of U.S. Pat. No. 4,458,066, which publications are each incorporated herein by reference. Synthetic procedures generally involve the sequential coupling of activated and protected nucleotide bases to give a protected nucleotide chain, whereafter protecting groups may be removed by suitable treatment. Preferably the compounds will be synthesized on an automated synthesiser such as those made by Applied Biosystems (a Division of Perkin Elmer), Pharmacia or Millipore.

In addition to being synthesized chemically, nucleic acid molecules with modified nucleotides may be synthesized enzymatically. The phosphodiester bonds of RNA can be replaced by phosphorothioate linkages by in vitro transcription using nucleoside α-phosphorthio triphosphates. T7 RNA polymerase specifically incorporates the Sp isomer of α-phosphorthiotriphosphate with inversion of configuration to produce the Rp isomer of the phosphorothioate linkage. The methods to produce transcripts fully substituted with phosphorothioate linkages adjacent to a given nucleotide, or to produce partially substituted transcripts containing approximately one phosphorothioate linkage per molecule, are described by Ruffner and Uhlenbeck (1990). Conrad et al. (1995) describe methods of using T7 RNA polymerase to produce chimeric transcripts containing ribonucleotides and deoxyribonucleotides (with and without phosphorothioate linkages), and also ribonucleotides and 2′-O-methylnucleotides (with and without phosphorothioate linkages). These methods have been shown to produce transcripts containing up to 50% deoxyribonucleotides, and up to 58% 2′-O-methylnucleotides. Aurup et al (1992) describe methods for using T7 polymerase to produce transcripts containing 2′-fluoro-2′-deoxyuridine, 2′-fluoro-2′-deoxycytidine, and 2′-amino-2′deoxyuridine. (Aurup, 1992; Conrad, 1995; Ruffner, 1990) Further means for producing the nucleic acid molecules of this invention are further discussed below (Sambrook, 1989).

Nucleotides represented in the compounds above comprise a sugar, base, and a monophosphate group or a phosphodiester linkage. Accordingly, nucleotide derivatives or modifications may be made at the level of the sugar, base, monophosphate groupings or phosphodiester linkages. It is preferred that the nucleotides in the compounds above be deoxyribonucleotides, ribonucleotides or RNA/DNA hybrids, however, other substitutions or modifications in the nucleotide, such as PNA, are possible providing that ability to hybridise is not lost.

Nucleotide bases, deoxyribonucleotide bases, and ribonucleotide bases are well known in the art and are described, for example in Principles of Nucleic Acid Structure (Saenger, 1984). Furthermore, nucleotide, ribonucleotide, and deoxyribonucleotide derivatives, substitutions and/or modifications are well known in the art (See, for example, Saenger, 1984; Sober, 1970), and these may be incorporated in the nucleic acid molecule made with the proviso that the ability to hybridise to complementary nucleic acid sequences is not lost.

In addition, a large number of modified bases are found in nature, and a wide range of modified bases have been synthetically produced (See, for example, Saenger, 1984; Sober, 1970). For example, amino groups and ring nitrogens may be alkylated, such as alkylation of ring nitrogen atoms or carbon atoms such as N1 and N7 of guanine and C5 of cytosine; substitution of keto by thioketo; saturation of carbon-carbon double bonds, and introduction of a C-glycosyl link in pseudouridine. Examples of thioketo derivatives are 6-mercaptopurine and 6-mercaptoguanine. Bases may be substituted with various groups, such as halogen, hydroxy, amine, alkyl, azido, nitro, phenyl and the like. The phosphate moiety of nucleotides or the phosphodiester linkages of oligonucleotides are also subject to derivatisation or modifications, which are well known in the art. For example, replacement of oxygen with nitrogen, sulphur or carbon gives phosphoramidates, (phosphorothioates, phosphorodithioates) and phosphonates, respectively. Substitutions of oxygen with nitrogen, sulphur or carbon derivatives may be made in bridging or non-bridging positions.

A further aspect of the invention provides alternative linkages such as an amide, carbamate, thiocarbamate, urea, amine, a sulfonamide, a hydroxylamine, a formacetal, a 3′-thioformacetal, a sulfide, allyl ether, allyl, ether, thioether, PNA (peptide nucleic acid) or an ethylene glycol function to replace the conventional phosphodiester linkage. These modifications may increase resistance towards cellular nucleases

Possible nucleotide modifications include sugar modifications such as 2′ fluoro, 2′ amino, 2′ O-allyl, 2′ C-allyl, 2′ O-methyl, 2′ O-alkyl, 4′-thio-ribose, arabinose, other sugars, or non-circular analogues.

Phosphate modifications may be phosphorothioate (non-bridging), phosphorodithioate (non bridging), 3′ bridging phosphorothioate, 5′ bridging phosphorothioate, phosphoramidates, 3′ bridging phosphoramidate, 5′ bridging phosphoramidate, methyl phosphonate, other alkyl phosphonates or phosphate triesters.

Modifications in base may be purine, 2,6-diaminopurine, 2-aminopurine, O⁶-methylguanosine, 5-alkenylpyrimidines, 5-propyne, inosine, 5-methylcytosine, pseudouridine, a-basic (ribose or deoxyribose).

Some nucleotides may be replaced with the following chemical linkers: 1,3-propane-diol, alkane-diols, or various polymers of (ethyleneglycol, tetraethylene glycol, hexaethyleneglycol).

Other Modifications to the 3′ end may be selected from: 3′-3′ inverted linkage (inverted diester or inverted phosphoramidate), 3′-3′ linked abasic ribose, or end-capped (methoxyethylamine phosphoramidate).

Modified sugars may be synthesized as follows: 2′-deoxy-2′-fluorouridine (Sinha, 1984); 2′-deoxy-2′ fluoro cytidine (Sinha, 1984); 2′-deoxy-2′ fluoroadenosine; synthesis and incorporation into nucleic acid molecule (Olsen, 1991); 2′-deoxy-2′-amino uridine and 2′-deoxy-2′-amino cytidine (Heidenreich, 1994); 2′-O-allyl-(uridine or cytidine or adenosine or guanosine) (Available from Boehringer Mannheim, Mannheim, Germany) or (Badger, 1994). 2′-deoxy-2′-C-allyl-ribonucleotides; 2′-O-methyl ribonucleotides see Review: (Sproat, B. S., 1991A) (also available from Chemgenes, Waltham, Mass. or Glen Research, Sterling, Va.), other 2′-O-alkyl-ribonucleotides, synthesis see (Monia, B. P., 1993; Sproat, B. S., 1991B); α-anomer of uridine, cytidine, adenosine and guanosine, see (Debart, F., 1992 and references therein); other modified sugars, etc. Arabinose (Garbesi, A., 1993); Hexose-thymidine (Augustyns, K., 1992) and linear analogues of sugars (Hendry, 1994).

Modified phosphates may be synthesized as follows: Phosphorothioate; synthesized by modification of oxidation procedure during phosphoramidite synthesis. Reagents commercially available from Perkin Elmer and others, products are mixture of isomers, some methods available for stereospecific synthesis of phosphorothioate, see ref: (Stec, 1991); Phosphorodithioate; (Eldrup, A. B., 1994; Caruthers, 1991; Beaton, 1991); 3′-bridging phosphorothioate; 5′ bridging phosphorothioate; phosphoramidates (non-bridging, oxidize the phosphite triester with solution containing the required amine); (Froehler, B., 1988; Jager, A., 1988; Letsinger, R. L., 1988); 3′ bridging phosphoramidate (NH replaces 3′ O) (Forms very stable duplexes) (Letsinger, 1992; Gryaznov, S. M., 1995; Chen, J. K., 1995); 5′ Bridging Phosphoramidate (NH replaces 5′ 0; thymidine analogue only, weak binder) (Gryaznov, S. M., 1992); Methylphosphonate (reagents are commercially available; Glen Research or Chemgenes Stereospecific; Rp isomers bind stronger: (Savchenko, 1994; Miller, 1991); 5′-deoxy, 5′-methylphosphonate (Szabo, 1995); Other alkyl-phosphonates (Fathi, 1994A; Fathi, 1994B); Phosphate triesters (Summers, 1986).

Replacements for the phosphodiester linkage may be synthesized as follows: For review see (De Mesmaker, 1995) Amides (Chur, 1993; Blommers, 1994; De Mesmaeker, 1993; De Mesmaeker, 1994A; De Mesmaeker, 1994B; Lebreton, 1993; Lebreton, 1994A; Lebreton, 1994B; Idsiak, 1993): Carbamate (Waldner, 1994; Stirchak, 1987; Habus, 1994; Thiocarbamate (Waldner, 1995); Ureas (Waldner, 1994) Amines (De Mesmaeker, 1994C; Caulfield, 1994); Hydroxylamine (Debart, 1992; Vasseur, 1992; Formacetal (Matteucci, 1990; Jones, 1993) Thioformacetal (Jones, 1993); Allyl ether (Cao, 1994); Allyl, Ether, Thioether (Cao, 1994); Alkane (De Mesmaeker, 1994; PNA A selection of binding and antisense properties (Nielsen, 1993A; Hanvey, 1992; Egholm, 1993; Nielsen, 1993B); PNA Synthesis (Egholm, 1992A; Egholm, 1992B); Preparation of purine PNA monomers and oligonucleotides (available commercially from Millipore corporation).

Modified bases may be synthesized as follows: Purine; synthesis and incorporation into nucleic acid molecule (Slim, 1992; Fu, 1992; Fu, 1993); 7-deazaGuanosine, synthesis and incorporation into nucleic acid molecule (Fu, 1993); Inosine, synthesis and incorporation into nucleic acid molecule (Slim, 1992; Fu, 1993) 7-deazaAdenosine, synthesis and incorporation into nucleic acid molecule (Fu, 1992; Seela, 1993). O⁶-methylguanosine, synthesis and incorporation into nucleic acid molecule (Grasby, 1993); 2,6-diaminopurine, synthesis (Sproat, 1991); 2-aminopurine, synthesis and incorporation into nucleic acid molecule (Ng, 1994; Tuschl, 1993); Isoguanosine, synthesis and incorporation into nucleic acid molecule (Ng, 1994; Tuschl, 1993); Xanthosine, synthesis and incorporation into nucleic acid molecule (Tuschl, 1993); 6-azathymidine, 6-aza-2′-deoxycytidine, synthesis and incorporation into oligonucleotides (Sanghvi, 1993); 5-alkenylpyrimidines; 5-propyne (Gilead, Froehler); inosine; 5-methylcytosine; pseudouridine; abasic ribose or deoxyribose.

As discussed above, once the nucleic acid molecules have been synthesised, or otherwise obtained, they may be immobilised onto the nanotube by a variety of methods including those methods normally used for coupling nucleic acids to solid supports. For references describing these methodologies, see Silman, I. H. and Katchalski, E. in Annual Review of Biochemistry, Vol. 35, p. 873 (1966); Melrose, G. J. H., in Review of Pure and Applied Chemistry, Vol. 21, p. 83, (1971); and Cuatrecasas, P. and Anfinsen, C. B., in Methods in Enzymology, Vol. 22, (1971). For example, nucleic acid molecules may be covalently or non-covalently associated with the carbon nanotubes of this invention.

Nucleic acid molecules can be attached to solid supports such as the surfaces of carbon nanotubes by diverse non-covalent interactions including simple non-covalent absorption driven by free energy changes of the system. However, pre-synthesised DNA molecules bound in this way lie flat against the surface of the nanotubes (Tsang, S. C. et al., 1997, Angew. Chem. Int. Ed. Engl., 36, 2198-2200) and the single strands of DNA are most likely unable to hybridise to their complementary DNA strands. Particularly advantageous procedures for physical attachment of nucleic acids to nanotubes, that will allow the physically-attached single-stranded nucleic acid to bind its complementary strand, involve firstly the physical absorption of a non-nucleotide or an oligonucleotide anchor to the nanotubes followed by chemical attachment of pre-made DNA to the anchor or build-up of the DNA from the physically bound anchor as described above.

Nucleic acid molecules can also be attached to solid supports such as carbon nanotubes by covalent coupling of the nucleic acid to the surface. Particularly advantageous procedures for chemical attachment of nucleic acids to nanotubes involve modifying the nanotubes by either direct functionalisation of nanotube tips or active sites on the nanotube walls and/or attachment of a functional linker to the tips and/or the walls of the nanotubes. Both of these methods result in covalent attachment of the nucleic acids to the nanotubes. It will be appreciated that the type of initial treatment and type of attachment are partially dependent upon the end use of the nanotube.

The attachment of the nucleic acid molecule on the tips or at active sites on the walls of the nanotubes generally requires the nanotubes to be functionalised to produce a free carboxyl or hydroxyl group.

The term “functionalised nanotube” is used in its broadest sense and refers to a nanotube having functional group(s) such as hydroxyl, carboxyl and/or aldehyde group(s).

The attachment of the nucleic acid molecule on the walls of the nanotubes i.e., along the sides or length of the nanotube requires the attachment of a linker, for example an azido compound, to the nanotube such that a free functional group such as hydroxyl, carboxyl, amine, hydrazine, aldehyde or a maleimide, can be provided for further growing the DNA in situ from that site or for chemical reaction with a pre-synthesised DNA molecule containing a functional group such as amine, carboxyl or sulfhydryl. The DNA molecule may be functionalised with these groups either on the 5′ or 3′ end, using 5′-modifier phosphoramidite or 3′-modified CPG available from Glen Research.

In the first method, nanotubes are functionalised by chemical treatment with nitric acid and sulphuric acid solution to produce hydroxyl and carboxyl functional groups (see for example: Liu et al., Science 1998, 280, 1253; Chen et al., Science 1998, 282, 95; Hamon et al., Adv. Mater. 1999, 11, 834; Sloan et al., Chem. Commun. 1998, 347.) For example, in one methodology, the nanotube is refluxed in HNO₃ and/or H₂SO₄ to open the nanotube tips and to introduce —COOH and —OH groups at the open ends.

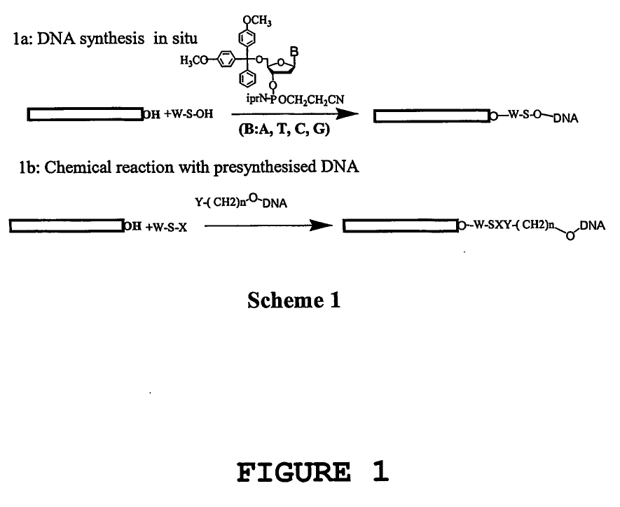

In one embodiment, free hydroxyl groups are predominantly produced (together with carboxyl groups) using milder acid conditions. The nucleic acid(s) are then built up in situ from the hydroxyl groups using the automated DNA synthesizer. In a further embodiment, the hydroxyl group can be reacted with functional group W on a difunctional spacer-molecule W—S—X of formula I as defined above, in order to extend the second functional group X further from the surface of the nanotube and thus allow for attachment of a DNA molecule to X with less steric hindrance from the nanotube. If X is a hydroxyl group, the nucleic acid molecules can be built up in situ using the automated DNA synthesizer (FIG. 1—Scheme 1a). Alternatively, if X is a different functional group such as carboxyl or amine, a pre-synthesised DNA containing a linker with functional group Y can be attached to the extended functional groups on the nanotubes by reaction of Y with X using methods known in the art (FIG. 1-Scheme 1b). For example, when X is a carboxyl group, Y may be an amine group. W—S—X may be, for example, succinic anhydride, or bromoacetic acid.

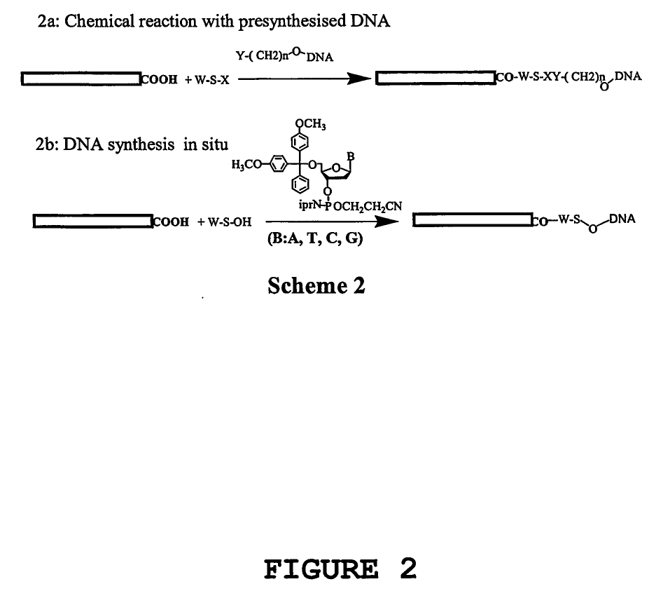

In another embodiment, nanotubes modified with carboxyl groups are attached to the nucleic acid molecule using reaction conditions compatible with carboxyl groups. The nucleic acid molecule may be presynthesised with a linker containing a functional group, for example, an amino linker using any suitable technique known in the art, and this may be covalently attached to the nanotubes via reaction of the amine group on the DNA with the carboxyl groups on the nanotubes to form an amide bond (FIG. 2—Scheme 2a). In a further embodiment, the carboxyl group can be reacted with functional group Wₐ on a difunctional spacer-molecule Wₐ—Sₐ—Xₐ of formula Ia as defined above, in order to extend the second functional group Xₐ further from the surface of the nanotube and thus allow for attachment of a DNA molecule to Xₐ with less steric hindrance from the nanotube. If Xₐ is a hydroxyl group, the DNA can be built-up in situ using phosphoramidite chemistry (FIG. 2—Scheme 2b). If Xₐ is some functional group other than hydroxyl, a pre-synthesised DNA containing functional group Yₐ can be attached to the functional group Xₐ on the nanotube using methods known in the art. (FIG. 2—Scheme 2a). If Xₐ is a carboxyl group, an amino-modified DNA can be used to react with this carboxyl group and form an amide bond, using any suitable known method, such as the hydroxy succinimide active ester method or the acid chloride method.

In the second method, a linker containing functional groups is attached to the walls of the nanotubes by a photochemical reaction. The linker is preferably an azido compound such as azido-thymidine or azidonitrobenzoyloxysuccinimide. It is attached by exposing the nanotube to the azido compound and then irradiating the exposed nanotube with UV light. The azido compound binds non-specifically to the nanotube and provides a free hydroxyl or carboxyl group.

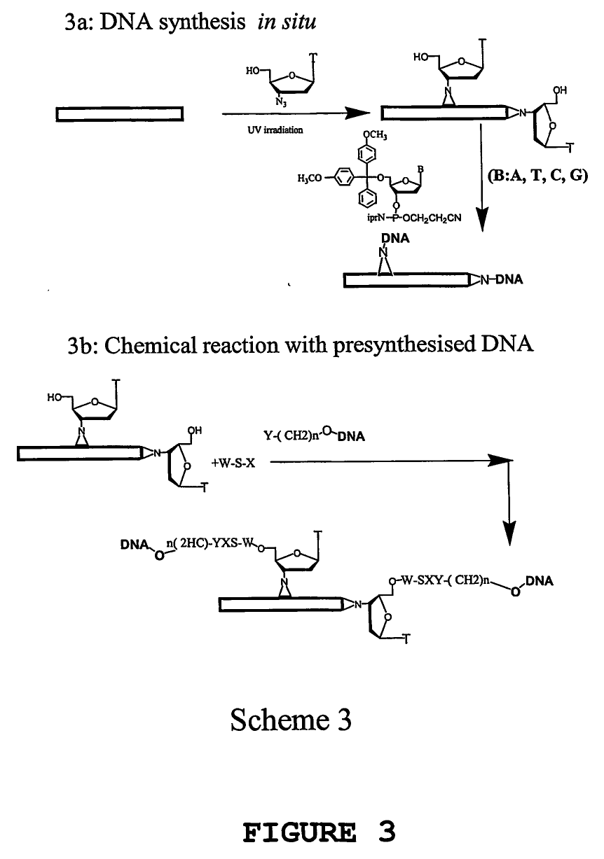

In one embodiment, the free hydroxyl groups on the 5′ position of the AZT provide sites from which nucleic acid molecules can be grown in situ using the automated DNA synthesizer (FIG. 3—Scheme 3a). In a further embodiment, the hydroxyl group can be reacted with functional group W on a difunctional spacer-molecule W—S—X of formula I as defined above, in order to extend the second functional group X further from the surface of the nanotube and thus allow for attachment of a DNA molecule (FIG. 3—Scheme 3b). If X is a carboxyl group, it may be reacted with the amine group of a pre-synthesised amino-modified nucleic acid molecule to form an amide bond using any suitable known method, such as the hydroxy succinimide active ester method or the acid chloride method.

In yet a further embodiment, the nanotubes are functionalised with linkers which are attached to the nanotubes by a photochemical reaction and which contain suitable functional groups including, carboxyl, or activated carboxyl, amine, isothiocyanate or maleimide, for example, azido nitrobenzoyloxysuccinimide, 4-(P-Azidosalicylamido) butylamine, Azidophenyl isothiocyanate, 4-(P-Azido salicylamido) butyl-3′ (2′-pyridylthio)propionamide, 4-(P-Azido salicylamido) butyl-maleimide propionamide, and p-azidophenyl glyoxal monohydrate.

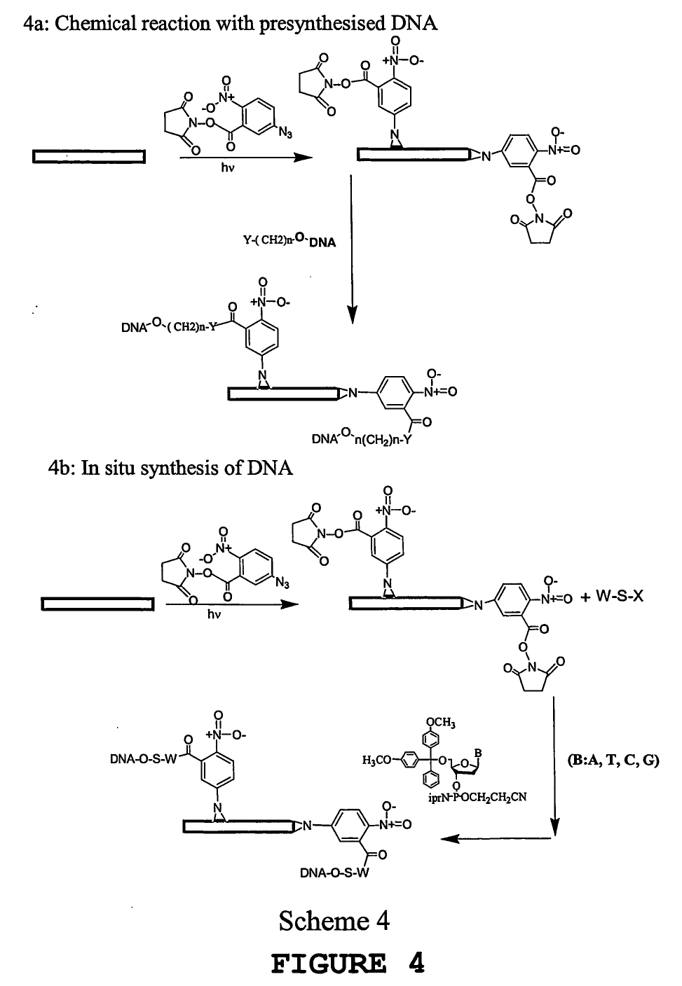

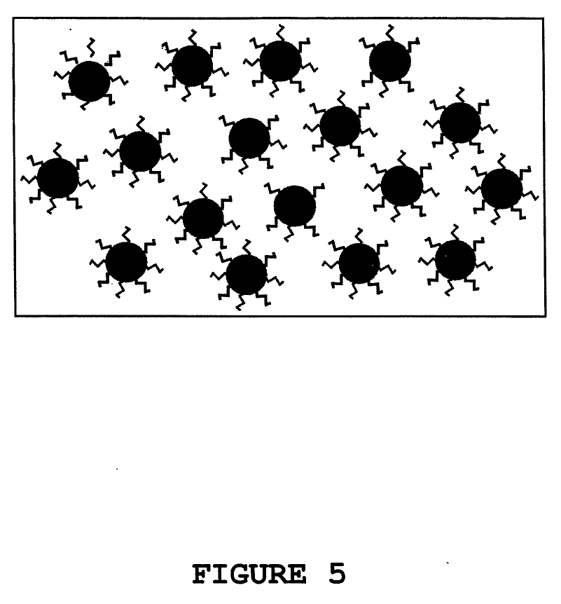

In a further embodiment, the nucleic acid molecule is then reacted with the modified nanotube using reaction conditions compatible with the functional group. This may require the nucleic acid molecule to be presynthesised so as to provide an amino linker or carboxyl linker or sulphydryl linker using any suitable known technique as described herein. For example, a nucleic acid molecule(s) having an amino linker can be covalently linked via an amide, urea or thiourea bond to nanotubes photoetched with azido nitrobenzoyloxysuccinimide, Azidophenyl isocyanate or Azidophenyl isothiocyanate groups, respectively (FIG. 4—Scheme 4a).

In yet a further embodiment, the photoetched functional group is reacted with a difunctional spacer to produce an hydroxyl functional group extended from the surface of the nanotube. For example, the difunctional group may be 2-chloroethanol. The nucleic acid(s) are then built up in situ in an automated DNA synthesiser from the hydroxyl group on the spacer (FIG. 4—Scheme 4b).

Once the desired nucleic acid molecule or molecules are attached to the nanotube, these are capable of being used in a number of ways. For example, if an oligonucleotide is chemically or physically attached to a nanotube either aligned or in random orientation, it may be used to capture a target DNA strand, if the target is substantially complementary to the DNA immobilised on the nanotubes. Two nucleic acid sequences are “substantially complementary” when at least about 85%, preferably at least about 90%, and most preferably at least about 95%, of the nucleotides or ribonucleotides are able to form base-pair matches (adenine with thymine, guanine with cytosine) over the defined length of the nucleic acid sequences. Sequences that are substantially complementary can be identified in a hybridization experiment, for example under “stringent conditions” as defined for that particular system. Defining appropriate hybridization conditions is within the skill of the art. See e.g., Sambrook et al., DNA Cloning, vols. I, II and III. Nucleic Acid Hybridization. However, ordinarily, “stringent conditions” for hybridization or annealing of nucleic acid molecules are those that

(1) employ low ionic strength and high temperature for washing, for example, 0.015M NaCl/0.0015M sodium citrate/0.1% sodium dodecyl sulfate (SDS) at ˜50° C. (the exact temperature will depend on the number of base pairs, with the optimum temperature being 1-2° C. below the melting temperature of the double helix which may be lower than 50° C. for short double helices), or

(2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Another example of “stringent conditions” is use of 50% formamide, 5×SSC (0.75M NaCl, 0.075M sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42° C., with washes at 42° C. in 0.2×SSC and 0.1% SDS.

The hybridisation event may be measured by a change in the electrical or electrochemical properties of a DNA molecule. Patterned or clustered nanotubes, each with a DNA of a different sequence chemically attached, may be used to screen for the presence of several target DNA molecules. The DNA may be immobilised either on the surfaces of the nanotubes and/or the tips. Devices with this configuration could be used as DNA biosensors, DNA arrays or as chips for DNA computers.

In general a biosensor is an analytical device that combines the specificity of a biological sensing element with a transducer to produce a signal proportional to target analyte concentration. Nanotubes with attached nucleic acid molecules act as a receptor in biosensing for the detection of complementary nucleic acid strands. These biosensors would be useful in clinical applications, eg screening for the presence of bacterial or viral nucleic acids, in pharmaceutical applications, agricultural applications, food control, hygiene and environmental monitoring and forensic applications.

The signal detection can result from a change in mass, or reduction in conductivity, or from using electrochemical techniques such as cyclic voltammetry (Garnier et al. 1999, Synth. Met. 100, 89-94), chronopotentiometry (Wang et al. 1997, Biosensors & Bioelectronics, 12, 587-599), electrochemical impedence spectroscopy (Brett et al. 1999, Electrochim. Acta 44, 4233-4299) or by field effect transistor amplification (Souteyrand et al., 1997, J. Phys. Chem. 101, 2980-2985), photocurrent spectroscopy (Lassalle et al, 2001. Biosensors & Bioelectronics, 16, 295-303), from acoustic properties to be detected by piezoelectric quartz crystals (Ketterer et al., 2000, Sensors & Actuators, b 65 (73-75), potentiometric amperometric, or optical transducer such as surface plasmon resonance (Bier et al. Sensors & Actuators, 1997, b 38-39 (78-82)), resonant mirror (Watts et al. 1995, Anal. Chem. 57, 4283-4289, Buckle, et al. 1993, Biosensors and Bioelectronics, 8, 355-363) or raman spectroscopy (Vo-Dinh et al, 1994, Anal. Chem., 66, 3379-3383). The signal detection can result also from detecting fluorescinated DNA molecules or DNA-modified magnetic particles hybridizing to the target DNA.

The signals may be further amplified and processed like other biosensors, known in the art. Similar to other biosensors, DNA biosensors are usually in the form of electrodes, chips and crystals, and hence hybridization on a sensory surface is a solid phase reaction.

The requirements for an ideal detector include high specificity and high sensitivity using a protocol that can be completed in a relatively short time. Moreover, systems that can be miniaturised and automated offer a significant advantage over current technology, especially if detection is needed in the field.

The electrochemical methods of detecting hybridisation events use the principle of electrical circuit completion. It is well known that carbon nanotubes are conductors of electricity. Accordingly, nanotubes are capable of detecting minute changes in conductivity. A DNA modified carbon nanotube of the present invention will have a specific, measurable electrical conductivity profile. Once a hybridisation event has taken place the electrical conductivity profile will change. This change is capable of being detected. For example, International patent application No. WO02/03050 describes a system of detecting hybridisation using the completion of an electrical circuit. However, this patent application also describes the problems inherent in the detection of electrochemical changes. The conventional theory in the electrochemical methods is that it is essential for the reference electrode potential to be very stable and not be affected by chemical changes in the solution. By using carbon nanotubes these problems are avoided.

A further use of the invention described herein is the ability to place dispersed nanotubes in desired locations using the attached nucleic acid molecules as locators. In this case, DNA of complementary sequence to the DNA on the nanotubes is chemically or physically bound to the desired location by writing with DNA ink via pens such as AFM tips or ink-jet printers, or through patterning e.g. using micro- and nano-photolithographic methodologies. The nanotubes are then brought to these locations through DNA-DNA hybridisation. This configuration will have applications in the self-assembly of devices made from carbon nanotubes, including the self-assembly of electronic circuits and devices on the nanometer scale.

Nanotubes chemically or physically modified with DNA may be linked to other DNA-modified nanotubes through DNA-DNA hybridisation either directly via the DNA molecules attached to the nanotubes, or indirectly via bridging DNA molecules with a variety of configurations. The nanotubes may be aligned roughly in parallel, when DNA on the walls of the nanotubes links the nanotubes side-by-side, or when the DNA on the tips of the nanotubes links nanotubes end-to-end. Devices formed by side-by-side linkages may be used as actuators when the nanotubes are laid on a non-expanding substrate. Devices formed by end-to-end linkages, or small bundles of nanotubes linked side-by-side and end-to-end, may be used as nano-scale conductors or semi-conductors, more specifically components in nano-electronic applications, as replacements for damaged nerves in prosthetic applications, or as the bio-electronic interface in bio-electronic devices. Alternatively, the nanotubes may be linked at angles to each other, including at right-angles to each other, when combinations of nanotubes are used with DNA modifications on the surfaces, and on the tips. Such configurations could have applications as transistors or gated devices.

Although the invention has been described with reference to presently preferred embodiments, it should be understood that various modifications can be made without departing from the spirit of the invention. Moreover, the following examples are offered by way of illustration only and are not intended to limit the invention in any manner. All patent and literature references cited herein are expressly incorporated.

### EXAMPLE 1

### Chemical and Physical Attachment of DNA to Carbon Nanotubes

Nucleic acid molecules have been covalently attached to carbon nanotubes using a number of different methods. The different strategies involved (1) aligned nanotubes or dispersed nanotubes, with different functional groups (hydroxyl or carboxyl) introduced predominantly on the tips of the nanotubes by chemical reaction, (2) aligned nanotubes, dispersed nanotubes, or mats of nanotubes, with functional groups introduced on to the nanotubes by photochemical reaction, using photoreactive functional groups such as azidothymidine, azido nitrobenzoyloxysuccinimide. In both strategies, the nucleic acid may be attached either by DNA synthesis in situ or by covalent coupling of pre-synthesised and functionalised DNA molecules to the nanotubes.

(1.1) In situ DNA Synthesis on Aligned, multi-walled Carbon Nanotubes with Functional OH/COOH Groups.

Aligned, MWNTs were grown on a quartz substrate, by pyrolysis of iron(II) phthalocyanine (FePc) under Ar/H₂ at 800-1100° C. (Li, D.-C., Dai, L., Huang, S., Mau, A. W. H. and Wang Z. L. (2000) Chem. Phys. Lett, 316, 349-355). The exposed ends of the nanotubes were sputtered with gold to form a thin gold-foil coating; this gold-foil coating with nanotubes was lifted off the quartz substrate to reveal clean ends of nanotubes free from amorphous carbon deposits. The tips of the nanotubes were functionalised with OH/COOH groups by refluxing the nanotubes in a mixture of HNO₃/H₂SO₄/H₂O for 2-3 h. The nanotubes were washed with several changes of ultra-pure autoclaved water until the pH was >6. Nanotubes sitting on approximately 20-25 mm² of gold foil were then placed in the reaction column of an Applied Biosystems DNA synthesiser, and a DNA molecule of 16 nucleotides was built up on the nanotubes using the phosphoramidite method in accordance with the manufacturer's instructions. The base sequence of the DNA molecule synthesised on the nanotubes was as follows:

5′TAC GCG AAT TGC CAC T3′.

The DNA molecule was attached to the nanotubes at its 3′ end. The progress of the reaction was monitored by the detritylation reaction at each step.

Approximately the same amount of nanotubes without any functional groups was used as a control to determine if the DNA was synthesised by covalent attachment to nanotubes or just simply by physical adsorption of oligonucleotides on the nanotubes.

The DNA-modified nanotubes on gold foil, and the control nanotubes on gold foil, were transferred from the DNA-synthesiser column to a glass vial, and 1.4 mL ammonia solution was added. The glass vial was sealed and then heated at 50-55° C. for 8-10 hours. After cooling, the ammonia solution was removed, and the nanotubes were washed with several changes of ultra-pure, autoclaved water until the pH of the washes was <7. At no time in this procedure were the nanotubes allowed to become dry. The DNA-modified nanotubes were stored under water at 4° C.

To determine the amounts and nature of any DNA molecules that were in the supernatant, and hence not chemically bound to the nanotubes after the synthesis, the ammonia solution and washes were saved, combined, and then rotary-evaporated under vacuum to remove ammonia and concentrate the solution. The concentrated solution was treated with polynucleotide kinase and γ-[32]-P-ATP to attach a [32]-P-phosphate to the 5′ ends of any DNA molecules present in the washes. The mix was electrophoresed on a 15% polyacrylamide gel containing 7M urea, and the pattern was visualised by exposure on a Molecular Dynamics PhosphorImager. Typically the pattern revealed DNA molecules varying in length from 1-2 nucleotides through to full-length, which indicated that some DNA molecules were being synthesised from nucleotides adsorbed on the surface of the nanotubes, and that some or all of these were removed on treatment with ammonia and/or by repeated washing. A small amount of full-length DNA, relative to the total amount of DNA, was in the supernatant taken from the oxidized nanotube sample, whereas a much higher proportion of full-length DNA, relative to the total amount of DNA, was in the supernatant taken from the control nanotube sample. The smaller amount of full-length DNA relative to the total DNA in the supernatant was suggestive of chemical attachment of full-length DNA to the oxidized carbon nanotubes.

(1.2) Attachment of a Pre-Synthesised DNA to Carbon Nanotubes.

DNA with amino linker was made on an Applied Biosystems DNA synthesiser.

The sequence of the 16-nucleotide DNA molecule was:

(5′ NH₂-(CH₂)₆- TAC GCG AAT TGC CAC T 3′)
or
(5′ TAC GCG AAT TGC CAC T (CH₂)₇-NH₂ 3′).

The DNA with the 5′-amino linker was treated with TCA/DCM for 5 min while on the DNA synthesizer to remove the MMT protecting group from the 5′-amino group. The DNA with the 3′-amino linker was treated with a 50/50 mixture of piperidine/DMF for 2 h at RT to remove the Fmoc protecting group from the 3′-amino group. These molecules were left to sit in 1 mL of ammonia solution for 30 min at room temperature to release them from CPG beads.

(1.2a) 2.1 mg of dispersed, oxidized, multi-walled nanotubes (oxidized by refluxing in HNO₃) were suspended in 1.5 ml thionyl chloride and heated to 80° C. for 3 h. The thionyl chloride was then decanted and the nanotubes were washed thoroughly with anhydrous acetonitrile. 40 nmole of 16-mer DNA with an amino hexane linker on the 5′ end and partially deprotected bases was dissolved in 1.5 ml of anhydrous DMF and added to the acid chloride modified nanotube. 5 μl of DIEA was added to the reaction mixture to adjust the pH=9. The suspension mixture was shaken gently under nitrogen atmosphere at RT for 16 h. The DMF solution was decanted after centrifugation of the reaction mixture and precipitation of the nanotubes. The nanotubes were partly suspended very uniformly in DMF and most did not precipitate even at high-speed centrifugation.

The DNA-modified MWNTS were transferred with water to a glass vial. Since it was difficult to pellet the nanotubes, the following procedure was used when changing solutions. The nanotubes were centrifuged at 12,000 rpm for ˜10 minutes. Most of the water was removed, and then the nanotubes were washed with 700 μL ammonia solution. The nanotubes were again centrifuged at 12,000 rpm for ˜10 minutes. Most of this ammonia solution was removed, and replaced with 1.4 mL of fresh ammonia solution. The glass vial was sealed, and heated at 55° C. for 8-10 hours. The black-brown suspension, which formed above the nanotubes in the solid phase, was removed, and saved. The nanotubes were washed with several changes of ultra-pure, autoclaved water until the pH of the washes was <7. At no time in this procedure were the nanotubes allowed to become dry. The DNA-modified nanotubes were stored under water at 4° C.

Sequences of DNA molecules attached to carbon nanotubes were:

NT3′-NH₂3′: 5′ TACGCGAATTGCCACT-(CH₂)₇-NH₂ 3′ +
COOH-NANOTUBES (attachment of 3′ end of DNA to
nanotubes)
and
5′NH₂-NT3′: 5′ H₂N-(CH₂)₆-TACGCGAATTGCCACT 3′ +
COOH-NANOTUBES (attachment of 5′ end of DNA to
nanotubes).

(1.2b) 2.1 mg of dispersed, oxidized SWNTs (Iljin), which had been refluxed in HNO₃ for 3 hr, filtered, washed thoroughly with water and dried, were suspended in 0.6 ml DMF. 10 mg Disuccinimidecarbonate (DSC) dissolved in 0.4 ml DMF was added to the nanotube suspension and sonicated for 1.5 h while shaken gently under nitrogen atmosphere. The black/brown suspension was precipitated by centrifuging the mixture at 14000 rpm for 30 min. 0.5 ml fresh DMF was added to the precipitate, and the mixture was vortexed for 1 min and then centrifuged at 14000 rpm for extra 30 min.

A 16-mer DNA with an amino linker on the 3′ end synthesized by automated DNA synthesis using (Dimethoxytrityloxy-3 fluorenmethoxycarbonylamino-hexane-2-methoxysuccinoyl) long chain alkyl amino-CPG) was released from CPG by treatment in 28% ammonia solution for 20 minutes.

Sequence of DNA molecules attached to carbon nanotubes was:

NT3′-NH₂3′: 5′ TACGCGAATTGCCACT-(CH₂)₇-NH₂ 3′

25 nmol of this DNA with partially deprotected bases was dissolved in 0.5 ml of anhydrous DMF and added to the succinylated modified nanotube. 5 μl of DIEA was added to the reaction mixture to adjust the pH=9. The suspension mixture was shaken gently under nitrogen atmosphere at RT for 16 hr. The sample was more suspended in the DMF after the reaction. Centrifugation did not precipitate the whole nanotube sample. DMF was evaporated off and the water was added to the precipitate. A black suspension was formed, indicative of the attachment of DNA to the nanotubes. The water was removed by rotary evaporation and the DNA was treated in 28% ammonia solution at 55° C. for 8 h. The ammonia solution was removed and the nanotubes were washed three times with water. The samples were store at 4° C. under water.
(1.3) Photoetching of AZT on the Surface of the Nanotubes Followed by In Situ DNA Synthesis.

About 10-50 μg of azidothymidine (AZT) (dissolved in ETOH) was coated on to two samples of aligned, multi-walled nanotubes attached to a piece of gold foil of area about 25 mm². The solution was allowed to evaporate at room temperature. The samples were air-dried and then irradiated with 450 W medium pressure lamp with max output at around 254 nm for 5 mins (112 mW/cm²). The samples were then washed thoroughly with ETOH to remove all the unreacted AZT. The removal of the unreacted AZT was assessed by monitoring the wash solution by HPLC.

The AZT photoetched nanotubes were then placed on a DNA synthesiser as described above and a 16-nucleotide and a 35-nucleotide DNA molecule was synthesised by the phosphoramidite method. As described above, the blocking groups on the synthesised DNA were then removed with ammonia solution.

The DNA molecules were attached to the nanotubes through their 3′ ends. Sequences of DNA molecules attached to carbon nanotubes were:

| | |
|---|---|
| NT3′ (azidoT): | 5′ TACGCGAATTGCCAC(azidoT)- |
| NT3′T₁₈(azidoT): | 5′ TACGCGAATTGCCAC(T)₁₈(azidoT) |

(1.4) Photoetching of a Linker on the Surface of the Nanotubes Followed by Attachment of a Pre-Synthesised DNA Containing a Functional Group.

About 10-50 μg of ANB-NOS (dissolved in DCM) was coated on to aligned, multi-walled nanotubes attached to a piece of gold foil of area about 25 mm² or to a mat of unmodified SWNTs formed by filtration of a suspension of the SWNTs in DMF. The ANB-NOS solution was allowed to evaporate at room temperature. The samples were air-dried and then irradiated with 450 W medium pressure. The samples were then washed thoroughly with DCM to remove all the unreacted azido-linker. The photoetched nanotubes were then reacted with 5′-amino modified DNA (pre-treated with piperidine/DMF to remove the Fmoc group from the 5′ amine group and then deprotected partially for 20 min at RT in ammonia solution to release the DNA from the CPG). The reaction was carried out in a DMF solution for 16 h and the pH was adjusted to 9 by adding DIEA. After coupling, the supernatant was removed and the nanotubes were washed several times with DMF and water. The blocking groups on DNA were then removed by exposure to ammonia solution at 55° C. for 8 h.

The Sequence of the DNA molecule attached to carbon nanotubes was:

5′ NH₂-(CH₂)₆-TACGCGAATTGCCAC

(1.5) Physical Attachment of DNA to Carbon Nanotubes—In Situ DNA Synthesis on Aligned Multi-Walled Nanotubes.

Unmodified, aligned, multi-walled carbon nanotubes on a piece of gold foil approximately 20 mm² in area were placed in a reaction chamber on an Applied Biosystems DNA synthesizer. As a control, a similar amount of gold foil containing aligned, multi-walled carbon nanotubes, modified with azidothymidine by a photochemical reaction as described above, were placed in a second reaction chamber.

A 35-nucleotide DNA molecule (NT3′-T19) was synthesised in each reaction chamber by the phosphoramidite method. As a further control for the physical attachment, a 16-nucleotide DNA molecule (NT3′) was synthesized in an additional reaction chamber. As described above, the blocking groups on the synthesised DNA were then removed with ammonia solution. Sequences of DNA molecules physically attached to carbon nanotubes were:

5′ TACGCGAATTGCCACTTTTTTTTTTTTTTTTTT 3′
and
5′ TACGCGAATTGCCAC 3′

Sequence of DNA molecules chemically attached to the azidothymidine-modified nanotubes was:

5′ TACGCGAATTGCCACTTTTTTTTTTTTTTTTTT(azidoT).

After deblocking, the supernatant ammonia solution and subsequent rinses with water were pooled, rotary-evaporated to dryness under vacuum, redissolved in water, labeled with P³², electrophoresed on a 15% polyacrylamide gel containing 7M urea, and imaged on a Molecular Dynamics PhosphorImager. The image revealed that DNA ranging in length from 1-2 nucleotides to full-length material was in the ammonia solution and/or water rinses from both reaction chambers. This implied that DNA was synthesized in each reaction vessel by physical absorption on to the nanotubes, and that some of this physically adsorbed DNA, if not all, could be removed by washing. Tests to determine if any physically adsorbed DNA remained attached to the nanotubes, and to see if it was in a state where it could bind its complementary strand, are described below in Example 4.
(1.6) Oxidisation of SWNTs Followed by DNA Attachment on the Tips.

SWNTs (10 mg) were oxidised in a concentrated H₂SO₄/30% H₂O₂ (5 ml) aqueous solution for 30 min to form carboxylic acid groups at the nanotube tips and defects on sidewalls. The sample was then diluted with 250 ml of distilled water. The pH of the solution was adjusted to neutral by adding 10 mM sodium hydroxide solution to the aqueous nanotube suspension. Activation of carboxylic acids was carried out by sonicating (0.003 mg of SWNTS in 100 μl of water) by adding 1-ethyl-3-(3-dimethyl amino-propyl) carbodiimide (EDC) (50 mM), for 1 h at room temperature. Following the activation step, the nanotube suspension was centrifuged at 14,000 rpm for 10 minutes to remove the excess EDC. 50 μl of 10 mM sodium phosphate buffer was added to the precipitate and the pH was raised to 8.0. Then 50 μl of 1.346 mM amino-modified oligonucleotides (NT3′-NH₂3′) with the sequence:

5′TACGCGAATTGCCACT-(CH₂)₇-NH2 3′

was added to the SWNT suspension. The suspension was shaken gently for 3 h at room temperature. The suspension was washed, and then centrifuged. The unreacted DNA solution was removed, followed by several washes with deionized water.
(1.7) Functionalisation of SWNTS on the Sidewalls by Photoetching of ANB-NOS in a Chlorobenzene Suspension.

1 mg of SWNTs (Rice University) was suspended in chlorobenzene and was sonicated for 5 h. 3 mg of ANB-NOS, dissolved in 0.2 ml of chlorobenzene, was added to the suspension and mixed thoroughly by sonication/and vortexing the sample for a further 30 min. The homogenous suspension was then transferred to a quartz cuvette filled with nitrogen and sealed tightly. The cuvette was placed at a 5 cm distance from a 450 W medium pressure mercury lamp with the intensity of 30 mW/cm², and irradiated for 15 min. The sample was then filtered through an Isopore membrane with pore size around 50 nm. The SWNTs mat formed by filtration was washed thoroughly with chlorobenzene and then ethanol and dried under air.

A control sample was prepared by filtering a suspension of unmodified SWNTs in chlorobenzene without derivatisation or UV irradiation and filtering the sample through isopore membrane to form a SWNTs mat.

(1.8) Functionalisation of Oxidised SWNTS on the Sidewalls by Photoetching of ANB-NOS in a DMF Suspension Followed by Attachment of DNA.

0.1 mg of dispersed, oxidized SWNTs (Rice University), which was suspended in 1 ml of water, was resuspended in 1 ml DMF by the following process. The suspension in water was centrifuged at 14000 rpm for 30 min and the supernatant was removed. This process was repeated 5 times, adding 1 ml fresh DMF each time in order to replace the water with DMF.

ANB-NOS (2 mg) dissolved in 100 μl of DMF, was added to 900 μl DMF suspension of SWNTs, and vortexed for 1 min to obtain a homogenous suspension. The suspension was then transferred to a quartz cuvette filled with nitrogen and sealed tightly. The cuvette was placed at a 15 cm distance from a 300 W high pressure mercury lamp with the intensity of 112 mW/cm², and irradiated for 10 min. The suspension turned to a dark orange-brown solution. The suspension was transferred to an eppendorf tube and centrifuged at 14000 rpm to precipitate the nanotubes. The supernatant was removed and the precipitate was resuspended in fresh DMF, vortexed and centrifuged. This process was repeated 3 times to remove all the unreacted ANB-NOS. The precipitate was then resuspended in 300 μl DMF (6.6 mg/ml).

A 35-mer DNA with an amino linker on the 3′end (NT3′-T₁₉-NH₂-3′) of the sequence TACGCGAATTGCCAC-T₁₉-(CH₂)₇—NH₂ 3′ (50 μl at 53.4 μM) was added to 20 μl of ANB-NOS derivatised SWNTs and the pH of the suspension was adjusted to 8-9 by adding 50 μl of NaHCO₃/Na₂CO₃ (0.1 mM). The suspension was shaken gently for 18 h at room temperature.

The suspension was precipitated by centrifuging the mixture at 14000 rpm for 30 min. 100 μl of deionised water was added to the precipitate and the suspension was mixed thoroughly. This process was repeated 3 times to remove the unreacted DNA.

(1.9) Patterning of DNA on Nanotubes: Patterning of ANB-NOS on Aligned MWNTs Followed by Attachment of DNA to ANB-NOS.

About 0.5 mg of ANB-NOS (dissolved in 25 μl of acetonitrile) was coated on to aligned multi-walled nanotubes attached to a piece of gold foil of area about 50 mm². The solution was allowed to evaporate at room temperature to obtain a dry film of ANB-NOS covering the nanotube walls. A mask was used to cover partly some area of the nanotubes and expose the non-covered area of the nanotubes to UV irradiation. The sample was then irradiated with 300 W high pressure mercury lamp at 112 mW/Cm² for 5 min. After irradiation, the sample was washed thoroughly with acetonitrile to remove all the unreacted ANB-NOS.

A 16-mer DNA with an amino linker on the 3′end (NT3′-NH₂ 3′) of the sequence TACGCGAATTGCCACT-(CH₂)₇—NH₂ 3′ (50 μl at 104 μM) was added to the nanotube sample and the pH of the suspension was adjusted to 8-9 by adding 50 μl of NaHCO₃/Na₂CO₃ (0.1 mM). The DNA solution covered the whole nanotube sample during the DNA coupling. The suspension was shaken gently for 20 h at room temperature.

After the coupling, the supernatant was removed and the sample was washed several times with deionised water and finally left in 100 μL of water.

### EXAMPLE 2

### Synthesis and Manipulation of DNA Molecules for Attaching to the Nanotubes and for Use in Assays to Determine the Status of DNA Attached to Nanotubes

DNA molecules were synthesised on an Applied Biosystems DNA synthesiser using the phosphoramidite method. The CPG-beads, with DNA attached, were tipped from the DNA-synthesiser columns into a glass vial, and 1-1.4 mL of ammonia solution was added. The glass vial was sealed, and heated at 55° C. for 8-10 hours. After cooling, the solution was transferred to a round-bottom flask, rotor-evaporated under vacuum to remove ammonia, and co-evaporated twice with autoclaved, milliQ water. The DNA was transferred with autoclaved water to an eppendorf tube, concentrated with sec-butanol, washed with ether, and pelleted on dry ice in 0.3M sodium acetate and 80% ethanol. The pellet was washed with 80% ethanol, dried under vacuum, redissolved in water, and stored frozen at −20° C.

The purity of each DNA oligomer was checked by labelling the 5′ end with [32]-P, using γ-ATP-[32]P and polynucleotide kinase, and electrophoresing the material on a 15% polyacrylamide gel containing 7M urea. If the full-length DNA oligomer was less than 98% pure, the oligomer was purified by electrophoresis on a preparative polyacrylamide gel containing 7M urea; gel slices containing the full-length molecule were excised, crushed, and soaked in water for 24 hours. The supernatant containing DNA was extracted, concentrated with sec-butanol, washed with ether, extracted twice with phenol-chloroform, washed with ether, and pelleted on dry ice in 0.3M sodium acetate and 80% ethanol. The pellet was washed with 80% ethanol, dried under vacuum, redissolved in autoclaved, milliQ water, and stored frozen at −20° C.

Concentrations of DNA oligomers were determined by measuring the absorbance at 260 nm and molar extinction coefficients (L. Mole⁻¹ cm⁻¹) of 15400 for Adenosine, 11700 for Guanosine, 7300 for Cytidine, and 8800 for Thymidine.

Sequences of functionalised DNA molecules synthesized for covalent attachment to nanotubes:

5′NH2-NT3′: 5′ NH₂—(CH₂)₆-TACGCGAATTGCCACT

NT3′-NH23′: 5′ TAC GCG AAT TGC CAC T (CH₂)₇—NH₂ 3′

NT3′-T19′NH23′: 5′ TAC GCG AAT TGC CAC (T)₁₉ (CH₂)₇—NH₂ 3′

Sequences of DNA molecules used for assays were:

NT3′:
5′ TACGCGAATTGCCACT 3′
NT3′antisense:
5′ AGTGGCAATTCGCGTA 3′
5′ AGTGGCAATTCGCGTACGGGGCCCCG 3′
Gold2A-SH3′:
5′ AGTGGCAATTCGCGTA-(CH₂)₃-S-[S-(CH₂)₃-OH] 3′
5′ CGGGGCCCCGAGTGGCAATTCGCGTA 3′
Gold3A-SH3′:
5′ AGTGGCAATTCGCGTA(T)₁₉-(CH₂)₃-S-[S-(CH₂)₃-OH] 3′

### EXAMPLE 3

### Determination of DNA Attachment onto Nanotubes

X-ray Photoelectron Spectroscopy (XPS) assays the chemical composition of a 10 nm surface layer. Thus, XPS can be used to determine if DNA is attached to the surface of nanotubes, particularly by detecting the presence of phosphorus, and increased amounts of nitrogen. However, it is not sufficiently sensitive to distinguish between DNA that is chemically attached or strongly physically adsorbed, nor can it reveal if the attached DNA is in a conformation that is able to bind its complementary strand. In order to characterise the DNA-modified nanotubes, XPS (Kratos Ultra Imaging XPS spectrometer, Mg ka at 150 W) was carried out. The results of these analyses are shown in Tables 1-3. The data shown in Table 1 are for the samples prepared in Example 1.1. The data shown in Table 2 are for the samples prepared in Example 1.2a for the 5′-amino modified DNA. In Table 3, the “fully treated” sample, which produced DNA chemically attached to the nanotubes, was prepared as in Example 1.3, and samples B and D, which produced DNA physically adsorbed to the nanotubes, were prepared as in Example 1.5.

To investigate further the extent of photochemical linkage with changing the irradiation time, XPS analysis of unmodified nanotubes was compared to nanotube samples dosed with the same amount of AZT and photoirradiated with a 450 medium pressure mercury lamp (20 mW/cm²) for two different times, 3 min and 9 min (Table 4.

As shown in Tables 1-3, in all except one case, the percentage of O increased and the percentage of C decreased compared to the parent unmodified nanotube sample, as might be expected for DNA attachment to nanotubes; however the oxygen content of the unmodified nanotubes was unexpectedly high, and not constant, and so relative numbers of % P and % N are most likely to be more indicative than absolute numbers for % P, N, O, and C. In all methods used for attaching DNA to the nanotubes, the percentage of N and P increased compared to the parent unmodified nanotube sample, as would be expected for DNA attachment to nanotubes.

As shown in Table 3, the % N and % P increased in both control samples B and D, relative to the unmodified sample A, which was indicative of DNA physically adsorbed on the surface of these nanotubes. However, the fully-treated sample which should produce chemical attachment of DNA to the nanotubes had even higher % P and % N than samples B and D, indicative of chemical attachment and, possibly, some physical adsorption, to the nanotubes.

As shown in Table 4, the percentage of nitrogen element on the surface of the nanotubes has increased with increased irradiation time, which is consistent with the increase of C—N bond energy at 285.97 ev. These two results provide evidence for increasing amounts of AZT chemically attached to the surface of the nanotube with increased irradiation time. The increase of C═O bond energy at 287.97 eV is also consistent with carbonyl groups on the thymine base (on AZT) being attached to the surface.

Also to investigate the photochemical reaction of azide derivatives with SWNTS, two different samples were prepared by photoirradiating SWNTS in two different solvents, as discussed in Examples 1.7 and 1.8, respectively. The XPS data for Examples 1.7 and 1.8 are compared in Tables 5 and 6, respectively. The XPS analysis was carried out on a mat of SWNTs formed by filtering the photoetched suspension of the SWNTs onto an isopore membrane. Both samples were compared with the non-treated control samples, which were suspended in similar solvent systems and were made as a mat by filtering the suspension onto an isopore membrane.

In both photoirradiated SWNTs samples, the percentage of carbon was slightly decreased and the percentage of N was increased compared to the respective unmodified nanotube samples. There were other elements such as Fe, S, Cl, and Si in the sample which were somewhat reduced in both cases after irradiation and photoetching.

Table 5b shows the detailed analysis of XPS data for the sample prepared as discussed in Example 1.7. An additional nitrogen species at bond energy (BE)=406 appeared which is characteristic of the NO₂ group, and a substantial increase at BE=399.4, which is related to the N—C bond of the aziridine group attached to the nanotubes. Consistent with this increase in nitrogen element was the increase in Oxygen as well as the related carbon species (C2, C3, C4) related to C═O and C—O. All these findings support a successful photochemical attachment of ANB-NOS onto SWNT surfaces in solution.

TABLE 1
XPS Data to Determine the Presence of DNA on Oxidized,
Aligned Multi-Walled Carbon Nanotubes where the DNA was
Synthesized in situ, according to the Method Described in
Example 1.1. (Numbers shown % of each Element)

| | Non-Treated | DNA-Attached |
|---|---|---|
| C^{1s} | 76.89 | 73.5 |
| N^{1s} | 3.18 | 5.79 |
| P^{2p} | — | 0.83 |
| O^{1s} | 15.32 | 14.85 |

TABLE 2
XPS Data to Determine the Presence of DNA with 5′-Amine
Linker on Dispersed, Oxidized Multi-Walled Carbon
Nanotubes, where the Sample was Prepared according to the
Method Described in Example 1.2a.

| | Non-treated | DNA-attached |
|---|---|---|
| C^{1s} | 73.03 | 52.66 |
| N^{1s} | 2.41 | 3.90 |
| P^{2p} | — | 0.83 |
| O^{1s} | 23.5 | 35.24 |

TABLE 3
XPS Data to Determine the Presence of DNA Synthesised in
situ on Aligned Multi-Walled Carbon Nanotubes, according
to the Method Described in Example 1.3 (Fully Treated) and
Example 1.5 (Controls B and D).

| | | | | | Fully |
|---|---|---|---|---|---|
| | Control A | Control B | Control C | Control D | treated |
| C^{1s} | 91.86 | 86.09 | 76.97 | 83.16 | 79.96 |
| N^{1s} | 3.43 | 5.46 | 2.34 | 5.68 | 7.08 |
| P^{2p} | 0.02 | 0.70 | — | 0.76 | 1.07 |
| O^{1s} | 3.68 | 6.81 | 20.36 | 9.74 | 10.31 |

A: No treatment
B: No treatment, DNA synthesis
C: AZT added, washed off, no synthesis
D: AZT added, washed off, no irradiation, DNA synthesis
Fully treated: AZT added, irradiated, DNA synthesis

TABLE 4
XPS Analysis of Aligned Multi-Walled Carbon Nanotubes
Photoetched with AZT for Varying Times. (Numbers Show % of
Each Element)

| | Control | Sample A | Sample B |
|---|---|---|---|
| C^{1s} | 84.34 | 90.49 | 88.89 |
| N^{1s} | 1.20 | 1.87 | 3.13 |
| O^{1s} | 14.24 | 7.64 | 7.98 |
| C^{1s} (285.97 ev, C—N) | 3.62 | 4.98 | 5.41 |
| C^{1s} (286.57 ev, C—O) | 5.43 | 7.60 | 5.22 |
| C^{1s} (287.97 ev, C═O) | 2.76 | 3.53 | 4.57 |

Control: Aligned MWNts with no treatment
Sample A: Aligned MWNts treated with AZT and exposed to 3 min irradiation
Sample B: Aligned MWNts treated with AZT and exposed to 9 min irradiation

TABLE 5a
XPS data for Single wall nanotubes photoetched with ANB-
NOS in chlorobenzene solution prepared according to the
method described in Example 1.7 (numbers show % of each
element)

| | Non-treated | Photoethched |
|---|---|---|
| C^{1S} | 88.1 | 77.7 |
| N^{1S} | 1.6 | 4.17 |
| O^{1S} | 8.5 | 14.8 |

TABLE 5B
XPS data for single wall nanotubes photoetched with ANB-
NOS in chlorobenzene solution prepared according to the
method described in Example 1.7, X/C: atomic ratios
relative to C

| | |
|---|---|
| SWNT | SWNT |
| (Control) | (Modified) |

| | BE (eV) | X/C | BE (eV) | X/C |
|---|---|---|---|---|
| C^{1s} (C1) | 284.40 | 0.928 | 284.40 | 0.722 |
| C^{1s} (C2) | 285.92 | 0.053 | 285.69 | 0.202 |
| C^{1s} (C3) | 288.18 | 0.008 | 288.53 | 0.058 |
| C^{1s} (C4) | 291.12 | 0.011 | 291.11 | 0.018 |
| O^{1s} (O1) | 531.62 | 0.059 | 532.12 | 0.150 |
| O^{1s} (O2) | 533.18 | 0.035 | 533.74 | 0.037 |
| O^{1s} (O3) | 536.40 | 0.003 | 536.60 | 0.003 |
| N^{1s} (N1) | 399.85 | 0.016 | 399.93 | 0.036 |
| N^{1s} (N2) | 402.24 | 0.002 | 401.67 | 0.012 |
| N^{1s} (N3) | | | 405.83 | 0.006 |
| CL^{2P} (CL1) | 197.89 | 0.002 | 197.80 | 0.002 |
| CL^{2P} (CL2) | 200.47 | 0.005 | 200.50 | 0.026 |
| FE^{2p} | 708.5 | 0.006 | 707.5 | 0.005 |
| S^{2p} | 168.2 | 0.003 | 167.7 | 0.001 |

TABLE 6
XPS data for single-wall nanotubes photoetched with ANB-
NOS in DMF solution, prepared according to the method
described in Example 1.8. Numbers show % of each element.

| | Non-treated | Photoetched |
|---|---|---|
| C^{1S} | 80.041 | 77.893 |
| N^{1S} | 1.259 | 4.028 |
| O^{1S} | 18.681 | 17.779 |

### EXAMPLE 4

### Assays to Quantify Amounts, and Locations, of DNA Attached to the Nanotubes and to Determine if the Attached DNA is in a Functional State for Hybridization

It is possible that a DNA molecule that is chemically attached at one end to carbon nanotubes may be lying strongly adsorbed on the nanotubes at its “free” end, and hence not be in a conformation that is able to bind to its complementary DNA strand when this is added. We have developed two assays to determine if the chemically attached DNA is in a useful state. The ³²P assay is easier to perform, and also produces quantitative data on the number of DNA molecules of complementary sequence which are hybridized to the DNA molecules attached to the nanotubes. The gold-nanoparticle assay is a striking visual assay that reveals the sites of attachment of the bound DNA molecules, and it also produces a minimal estimate of the numbers of DNA molecules that have bound their complementary strands.

³²p Assay

This assay tests if the DNA, which is chemically attached to the carbon nanotubes, is able to hybridise to its complementary strand and also is able to distinguish its complementary strand from other DNA molecules.

Complementary, and non-complementary, DNA molecules, with radioactive (³²P) phosphate groups attached to their 5′ ends, were added to the DNA-modified nanotubes, and unmodified nanotubes as controls. The amount of radioactivity remaining on the nanotubes after extensive washing permits the determination of the numbers of added DNA molecules which are bound to DNA molecules chemically attached to the carbon nanotubes, with corrections for non-specific binding.

Attaching the radioactive label to the DNA. 80 pmol of DNA oligomer were added to 1 μl polynucleotide kinase and 4 μl γ-[³²P]-ATP, in a total volume of 20 μl kinase buffer, and the mixture was allowed to react for 30-45 minutes at 37° C. The reaction was stopped with EDTA. The mixture was extracted twice with phenol-chloroform, washed once with ether, and then the DNA was pelleted from 0.3M sodium acetate, 80% ethanol, on dry ice. The DNA pellet was washed with cold 80% ethanol, dried under vacuum, dissolved in 80 μl autoclaved milliQ water to form a 1 μM solution and then stored frozen at −20° C. The base sequence of DNA molecules chemically attached to carbon nanotubes by the azidothymidine method was either:

5′ TACGCGAATTGCCACT (NT3′)
or
5′ TACGCGAATTGCCACTTTTTTTTTTTTTTTTTTTT (NT3′-T₁₉).

The base sequences of DNA molecules ³²P-labelled on their 5′ ends were:

5′ AGTGGCAATTCGCGTA (NT3′AS)

the antisense strand. NT3′AS has the base sequence which is complementary to the DNA attached to the nanotubes, and so it should bind to the DNA on the nanotubes if the latter is in a functional state.

5′ TACGCGAATTGCCACT (NT3′)

the sense strand. NT3′ has the same base sequence as the DNA attached to the nanotubes, and so it should not bind to the DNA on the nanotubes.
Experiment A. Determining the Extent of Specific and Non-Specific Binding of 5′-End ³²P-Labelled DNA to DNA-Modified Multi-Walled, Aligned Carbon Nanotubes.

Samples 1 and 2 are aligned MWNT on gold foil, to which DNA (NT3′-T₁₉) is chemically attached by the azidothymidine method. Samples 3 and 4 are unmodified, aligned MWNT on gold foil. 5′-end ³²P-labelled DNA molecules (NT3′AS, or NT3′) were added to the nanotube samples as indicated below.

| | NT3′ AS | NT3′ |
|---|---|---|
| Nanotubes-T₁₉-NT3′ | sample 1 | sample 2 |
| Nanotubes | sample 3 | sample 4 |

Method: The DNA-modified carbon nanotubes were stored under sterile water at 4° C. Prior to starting the experiment, all samples of nanotubes were soaked in several changes of sterile water at 37° C.

Water on all nanotube samples was replaced over a period of 1 hour with 3 changes of 50-100 μl 2×SSC, 0.1% SDS at 37° C. Following this equilibration, 5 μl of 1 μM ³²P-labelled DNA solution was added (NT3′AS to samples 1 and 3, and NT3′ to samples 2 and 4), and the samples were gently rocked for 5 hours at 37° C. The supernatant containing unbound ³²P-labelled DNA was removed, and the samples were washed with 2×SSC, 0.1% SDS, until no counts could be detected in the washings (4-6 washings).

The samples were transferred on to a glass plate and were covered with plastic film (Saran wrap). The samples were exposed to a PhosphorImager screen, and counts were quantified on a Typhoon 8600 Variable Mode Imager (Molecular Dynamics) using Image Quant software and Storage Phosphor mode.

To account for differential ³²P labeling of NT3′ and NT3′AS, the counts per NT3′ and NT3′AS molecules were quantified on the Phosphor Imager from stock solutions with known concentrations. The area of each nanotube sample was measured under an optical microscope. Sample sizes varied from 4 to 8 mm². The total counts for each sample were then converted to nanomoles of ³²P-labelled DNA per mm², and are given in Table 7. Note that some of the edges of the nanotube samples were jagged, and so the measured areas are estimates. However, the numbers in Table 8 are sufficiently accurate to reveal trends.

Experiment B. Determining the Effect of Express Hyb on the Binding of 5′-End ³²P-Labelled DNA to DNA-Modified Multi-Walled, Aligned Carbon Nanotubes.

Samples 1 and 2 are aligned MWNT on gold foil, to which DNA (NT3′) is chemically attached by the azido Thymidine method. Samples 3 and 4 are unmodified, aligned MWNT on gold foil. 5′-end ³²P-labelled DNA molecules were added to the nanotube samples as indicated below.

| | NT3′ AS | NT3′ |
|---|---|---|
| Nanotubes-NT3′ | sample 1 | sample 2 |
| Nanotubes | sample 3 | sample 4 |

Method: The DNA-modified carbon nanotubes were stored under sterile water at 4° C. Prior to starting the experiment, all samples of nanotubes were soaked in several changes of sterile water at 37° C.

Water was removed from all carbon nanotube samples, and then ˜50 μl ExpressHyb (hybridization solution from Clontech (Palo Alto, Calif.)) were added to all samples. The samples were left to equilibrate with gentle rocking for 2 hours at 37° C.; the ExpressHyb was replaced twice with fresh solution during this period. Then 5 μl of 1 μM ³²P-labelled DNA solution was added (NT3′AS to samples 1 and 3, and NT3′ to samples 2 and 4), and the samples were gently rocked for 7 hours at 37° C. Unreacted ³²P-labelled DNA solution and ExpressHyb was removed, and the samples were washed 4 times with 2×SSC, 0.1% SDS, at which point no counts could be detected in the washings. The samples were transferred on to a glass plate and were covered with plastic film (Saran wrap). The samples were exposed to a PhosphorImager screen, and counts were quantified on a Typhoon 8600 Variable Mode Imager (Molecular Dynamics) using Image Quant software and Storage Phosphor mode.

To account for differential ³²P labeling of NT3′ and NT3′AS, the counts per NT3′ and NT3′AS molecule were quantified on the Phosphor Imager from stock solutions with known concentrations. The area of each nanotube sample was measured under an optical microscope. Sample sizes varied from 7 to 11 mm². The total counts for each sample were then converted to nanomoles of ³²P-labelled DNA per mm², and are given in Table 8 below. Note that some of the edges of the nanotube samples were jagged, and so the measured areas are estimates. However, the numbers in Table 8 are sufficiently accurate to reveal trends.

TABLE 7
Nanomoles/mm² of ³²P-labelled DNA (either NT3′ AS or NT3′)
remaining on aligned MWNT after hybridization and washing.

| | MWNT- | MWNT- | | |
|---|---|---|---|---|
| | DNA + | DNA + | MWNT + | MWNT + |
| | NT3′ AS | NT3′ | NT3′ AS | NT3′ |
| Experiment A (no | 172 | 95 | 138 | 193 |
| ExpressHyb) | | | | |
| Experiment B | 90 | 6 | 58 | 29 |
| (with ExpressHyb) | | | | |

The two columns on the right hand side of Table 8 show the extent of physical adsorption of DNA on unmodified MWNT. The presence of Express Hyb, as in Experiment B. reduces considerably the physical adsorption of DNA on unmodified nanotubes (138 compared with 58, 193 compared with 29).

Data in the last column (MWNT+NT3′) compared with those in the second column (MWNT-DNA+NT3′, where the attached NT3′ on the nanotubes should not hybridise to the added NT3′) indicate that physical adsorption is significantly reduced when the nanotubes are modified with DNA (193 compared with 95, and 29 compared with 6), and that this is further reduced, substantially, when Express Hyb is also present (95 compared with 6, column 2).

Thus, there is very little physical adsorption, or non-specific binding, of DNA molecules to DNA-modified nanotubes in the presence of Express Hyb.

Hence, data in the first column of Table 8 indicate that NT3′AS is binding specifically to the DNA (NT3′T19 or NT3′) which is chemically attached to the nanotubes. This specific binding is further enhanced, relative to the low remaining level of non-specific binding, when Express Hyb is present (compare column 1 (90) and column 2 (6) in Experiment B).

Thus, specific binding of the DNA target strand to its complementary DNA strand which is chemically attached to carbon nanotubes is 15-fold higher than the level of non-specific binding, in the hybridizing conditions used here (i.e. in the presence of Express Hyb).

Visual Assay Using DNA-Modified Gold Nanoparticles and Transmissiom Electron Microscopy (TEM)

We have several means of determining if DNA is attached to carbon nanotubes, and if that DNA is in a functional state. For example, analysis by XPS indicates if the DNA is present on the surface of the nanotubes either by physical adsorption or chemical attachment. Analysis with radioactively-labeled DNA, with a base sequence complementary to that of the DNA strands bound to the nanotubes, indicates if the bound DNA is able to bind its partner strand and how much is bound.

It would also be highly useful if we could see exactly where the DNA is chemically attached on the nanotubes, since for some purposes we would like DNA to be on the tips of the nanotubes (e.g. for end-to-end linkages) and for others we would like DNA to be on the sides (e.g. for maximum DNA loading for sensors, or for side-by-side linkages of nanotubes). Therefore we have developed a visual assay using gold nanoparticles and TEM. For this assay, DNA molecules, with base sequence complementary to those attached to the nanotubes, are synthesized with a terminal linker containing a di-thiol group, through which chemisorption to gold nanoparticles occurs. The DNA strands on these modified gold nanoparticles bind to their partner DNA strands on the nanotubes, and thus bring the gold nanoparticles very close to the site of DNA attachment on the nanotubes. The locations of the gold nanoparticles are readily imaged by TEM.

In the following experiments, the base sequence of DNA molecules bound through the dithiol group to gold nanoparticles is either:

5′ AGTGGCAATTCGCGTA-(CH₂)₃-S-[S-CH₂)₃-OH] 3′ (Gold2A-SH3′)
or
5′ AGTGGCAATTCGCGTATTCATCCTCAACAT-(CH₂)₃-S-[S-CH₂)₃-OH] 3′ (Gold3A-SH3′).

The base sequence of DNA molecules chemically attached through their 3′ ends to carbon nanotubes is either:

5′ TACGCGAATTGCCACT-nanotube (NT3′)
or
5′ TACGCGAATTGCCACT(T)₁₈-nanotube (NT3′-T₁₉)
or
5′ TACGCGAATTGCCACT-(CH₂)₇-NH₂ (NT3′-NH₂3′).

The base sequence of Gold2A-SH3′ is complementary to that of NT3′ and NT3′-NH₂3′, and to the 16 nucleotides at the 5′ end of NT3′-T₁₉. The base sequence of the 16 nucleotides at the 5′ end of Gold3A-SH3′ is complementary to that of NT3′ and NT3′-NH₂3′, apd to the 16 nucleotides at the 5′ end of NT3′-T₁₉.

The following procedure prepares gold nanoparticles of diameter ˜14 nm.

All glassware (500 ml conical flask and 250 ml storage bottle) and Teflon stirrer were first washed in aqua regia (HCl:HNO₃=3:1 by volume), and rinsed very well with milliQ water.

200 ml of milliQ water in a clean 500 ml conical flask was heated and gently stirred with a clean Teflon magnetic stirrer. While the water was being stirred and heated, 2 ml of 1% (W/V) gold chloride solution was added. When the solution was boiling vigorously, 5 ml of 1% (W/V) sodium citrate was added as quickly as possible. The solution turned from pale yellow to colourless. The heat was reduced, but the solution was kept on the boil. Over a period of several minutes, the solution changed colour from clear to dark blue, to purple, to red. Once the solution was red, the heat was further reduced and the solution was kept boiling for about 10 minutes.

The solution was removed from the heat, and allowed to cool. A cover over the opening of the conical flask prevented dust contamination.

The average diameter and the size distribution of the gold nanoparticles was determined by imaging using a Transmission Electron Microscope. Typically, the diameter was approximately 16(±1) nm.

Determining the concentration of a solution of gold nanoparticles
Extinction coefficient (at 520 nm)=2.4×10⁸ M⁻¹ cm⁻¹
Concentration (M)=A_{(˜520nm)}/extinction coefficient

Typically, the undiluted sample of a solution of gold nanoparticles, made by the above procedure, has an absorbance maximum of ˜1.2 at 519 nm. So, a typical concentration is ˜5 nM.

Attachment of Single-Stranded DNA to Gold Nanoparticles

Small-scale preparation (to make ˜700 μl of 17 nM DNA-modified gold nanoparticle solution)

This procedure essentially follows that described by Storhoff, J. J., Elghanian, R., Mucic, R. C., Mirkin, C. A., and Letsinger, R. L. (1998) J. Am. Chem. Soc. 120, 1959-1964.

2.2 ml of a 5.0 nM solution of gold nanoparticles were mixed with 9680 pmol (40 μl of a 242 μM solution) of Gold2A-SH3′ in a Teflon tube (Nalge). The tube was wrapped in aluminium foil to keep out light.

The solution in the Teflon tube was rocked very gently overnight (˜19-22 hours). At this stage the colour of the solution was red. Then, 26 μl of 1M sodium phosphate buffer (pH 7.0) and 260 μl 1.0M NaCl solution were added to the DNA and gold nanoparticle solution in the Teflon tube (to make approximate final concentrations of 10 mM buffer and 0.1M NaCl), and the solution was again gently mixed for 48 hours.

The solution was centrifuged at 12,000 rpm for 30 minutes. The colourless supernatant was removed leaving a small volume of dark-red solution containing the DNA and gold nanoparticles. This dark red solution was washed with 1.6 ml of 0.1M NaCl, 10 mM phosphate buffer, and centrifuged at 12,000 rpm for 30 minutes. Again the colourless supernatant was removed. The washing, centrifuging, and removal of supernatant was repeated.

640 μl of 0.3M NaCl, 10 mM phosphate buffer, 0.01% azide was added to the dark red solution to make ˜17 nM solution of DNA-modified gold nanoparticles.

Large-scale preparation (to make ˜3 ml of 17 nM gold nanoparticle solution where the gold nanoparticles are modified with varying quantities of DNA)

10 ml of 5 nM gold nanoparticles were mixed with:

17593 pmol (7.27 μl of 2.42 mM Gold2A-SH3′), DNA strands:Au particles=352 or

44044 pmol (18.2 μl of 2.42 mM Gold2A-SH3′), DNA strands:Au particles=880 or

88088 pmol (36.4 μl of 2.42 mM Gold2A-SH3′), DNA strands:Au particles=1762.

After gently mixing overnight, 110 μl M sodium phosphate buffer and 110 μl 1M NaCl were added to the solution and the mixture was gently rocked for 48 hours. The solution was centrifuged at 12,000 rpm for 30 minutes. The colourless supernatant was removed leaving a small volume of dark-red solution containing the DNA and gold nanoparticles. This dark red solution was washed with 10 ml of 0.1M NaCl, 10 mM phosphate buffer, and centrifuged at 12,000 rpm for 30 minutes. The colourless supernatant was removed, and the washing, centrifuging, and removal of supernatant was repeated. The ˜0.3 ml of dark red DNA-modified gold solution was adjusted to ˜3 ml with 2.7 ml of 0.3M NaCl, 10 mM phosphate buffer, 0.01% azide solution to form a ˜17 nM Au (DNA modified) nanoparticle solution.

Visual Assay

This assay tests if the DNA which was attached to the carbon nanotubes was:

(i) able to hybridise to its complementary strand

(ii) able to distinguish its complementary strand from other DNA molecules; and

(iii) showed where the chemically attached DNA is located on the nanotubes.

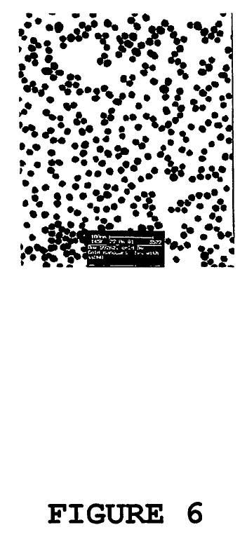

A schematic drawing of gold nanoparticles with single-stranded DNA molecules attached is shown in FIG. 5, and a TEM image of gold nanoparticles functionalised with DNA is shown in FIG. 6.

Complementary, and non-complementary, DNA molecules, bound to gold nanoparticles through dithiol groups on their 3′ ends, were added to the DNA-modified nanotubes, and unmodified nanotubes as controls. The locations of the gold nanoparticles on the nanotubes were visualized using Transmission Electron Microscopy.

Experiment C. Binding Gold Nanoparticles to Carbon Nanotubes Through 16-Nucleotide DNA Molecules.

Samples 1 and 2 were aligned MWNT on gold foil, to which DNA (NT3′) was chemically attached by the azidothymidine method. Sample 4 was unmodified, aligned MWNT on gold foil. Sample 3 was dispersed, oxidized nanotubes. Gold nanoparticles, or DNA-modified (Gold2A-SH3′) gold nanoparticles, were added to the nanotube samples as indicated in the table below.

| | Au-(Gold2A-SH3′) | Au |
|---|---|---|
| Nanotubes-NT3′ | sample 2 | sample 1 |
| Nanotubes | sample 4 | sample 3 |

Method: The carbon nanotube samples were equilibrated in ˜50 μl ExpressHyb (hybridization solution from Clontech (Palo Alto, Calif.)) at 50° C. for 30 minutes. The ExpressHyb solution was exchanged twice. Sample 3 appeared to swell in volume, with the nanotubes becoming very dispersed, and forming a brownish-black solution. The other samples had nanotubes attached to gold foil, and did not become dispersed.

50 μl of 5 nM gold nanoparticle solution was added to each of samples 1 and 3, and 25 μl of 17 nM DNA-modified gold nanoparticle solution was added to each of samples 2 and 4. The mixtures were vortexed, centrifuged, and left to slowly rock at 37° C. for 20 hours.

Pale red supernatants containing free gold nanoparticles or free DNA-modified gold nanoparticles were removed from samples 1, 2 and 4. The samples were washed with 5 μl of 2×SSC, 0.1% SDS, centrifuged, and supernatants were again removed. This washing procedure was repeated twice more, at which stage supernatants were colourless. Finally, the samples were washed twice with 0.1M NaCl, 10 mM sodium phosphate buffer. Supernatants remained clear.

Sample 3 was centrifuged for 5 minutes. Most nanotubes settled to the bottom of the tube. The black-brown supernatant was removed and the remaining nanotubes were washed as described above, but with extended times for centrifuging.

50 μl 0.1M NaCl, 10 mM sodium phosphate buffer were added to all four samples, in preparation for imaging by TEM.

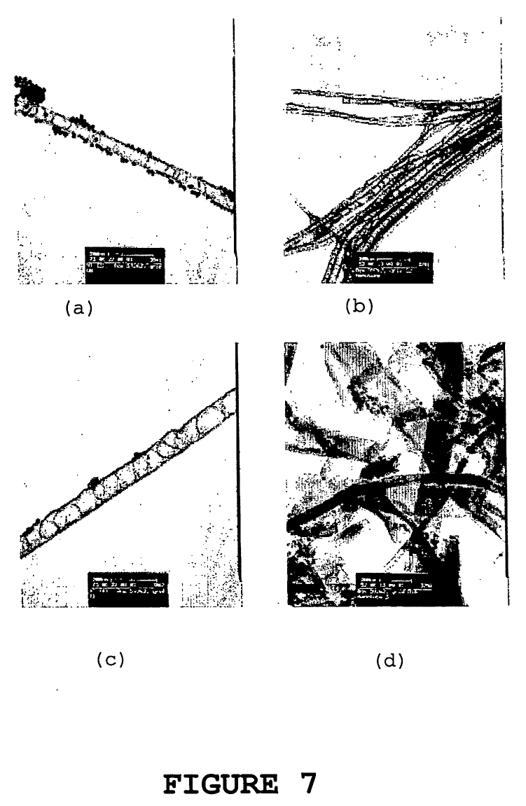

TEM images of each sample are shown in FIG. 7. Gold nanoparticles pepper the surfaces of nanotubes in Sample 2 (FIG. 7(a)), revealing that the azido-thymidine method results in chemical attachment of DNA over the surfaces of nanotubes, as expected. There are no gold nanoparticles on nanotubes in the control sample 1 (FIG. 7(b)), indicating that gold nanoparticles are not attracted to DNA-modified nanotubes under the experimental conditions used. Occasional nanotubes in control Sample 4 have a very small number of gold nanoparticles on their surface, indicating that DNA on the gold particle may be physically adsorbed on the nanotubes; one such nanotube is shown in FIG. 7(c). These gold nanoparticles do not seem to be as discrete as those in FIG. 7(a), but more blurred as if they have partially “melted”. There are gold nanoparticles scattered through Sample 3 (FIG. 7(d)) which consists of the oxidized nanotubes which formed a brownish-black mixture in ExpressHyb. These gold nanoparticles do not appear to be peppering the surfaces of nanotubes, as in Sample 2, but rather are mainly caught up in what appears to be amorphous material.

Experiment D. Binding Gold Nanoparticles to Carbon Nanotubes Using DNA Molecules Longer than 16 Nucleotides.

The above experiment was repeated, this time including longer DNA molecules on both the nanotubes and the gold nanoparticles. A T₁₉ (=TTTTTTTTTTTTTTTTTTT) extension on the DNA allows the remaining 16 nucleotides, which form the double helix and join the nanotubes to the gold nanoparticles, to extend away from the surface of the nanotubes, and hence further into solution. The experimental procedure was similar to that for Experiment C, except that the washings with 2×SSC, 0.1% SDS were replaced by three washings with 0.1M NaCl, 10 mM sodium phosphate buffer. The final washings were colourless.

Samples I, J and K are multi-walled aligned carbon nanotubes on gold foil, to which DNA (NT3′-T₁₉) is chemically attached by the azidothymidine method. Samples L, M and N are unmodified, aligned multi-walled nanotubes. Gold nanoparticles, or DNA-modified (either Gold2A-SH3′ or Gold3A-SH3′) gold nanoparticles, were added to the nanotube samples as indicated below. Note that the DNA molecules Gold2A-SH3′ and Gold3A-SH3′ have 16 and 30 nucleotides, respectively.

| | Au-Gold2A | Au-Gold3A | Au |
|---|---|---|---|
| Nanotubes-T₁₉-NT3′ | Sample I | Sample J | Sample K |
| Nanotubes | Sample L | Sample M | Sample N |

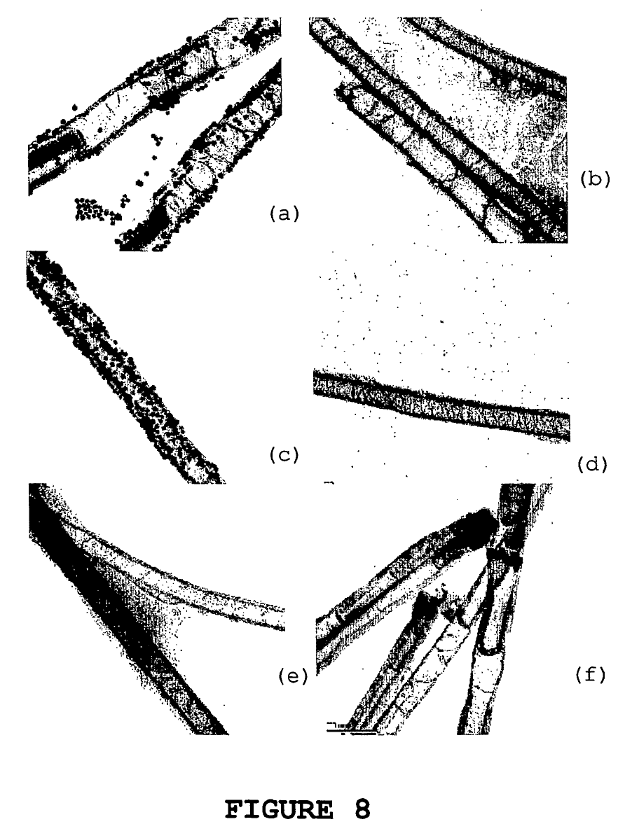

TEM images are given in FIG. 8. These show gold nanoparticles all over the surfaces of nanotubes in Samples I (FIG. 8(a) and J (FIG. 8(c), indicating that DNA on the gold nanoparticles is able to bind complementary strands which are chemically attached to the nanotubes. In the controls, there are no gold nanoparticles, indicating that gold nanoparticles are not attracted to DNA-modified nanotubes (Sample K, FIG. 8(e)), DNA-modified gold nanoparticles do not bind non-specifically (through physical adsorption) to unmodified nanotubes (Samples L (FIG. 8(b) and M (FIG. 8(d)), and gold nanoparticles do not attach to unmodified nanotubes (Sample N, FIG. 8(f)), under the experimental conditions used. The azido-thymidine method of chemical attachment of DNA to nanotubes results in DNA being placed all over the surfaces of the nanotubes, as indicated by the gold nanoparticles in Samples I and J. These results essentially confirm all of those obtained in Experiment C.
Experiment E. Binding DNA-Modified Gold Nanoparticles to DNA-Modified Single-Walled Carbon Nanotubes.

DNA (NT3′-NH₂3′, NT3′ with an amine linker on the 3′ end) was reacted with single-walled nanotubes (commercial sample from ILJIN) which had been functionalised with carboxyl groups on the tips. Using this reaction, the DNA was expected to become chemically attached to the tips of the nanotubes via an amide bond.

Approximately equal volumes of DNA-modified SWNT (Samples C and D) and un-modified SWNT (Samples E and F) were pipetted into eppendorf tubes, and excess water was removed. 50 μl ExpressHyb was added to each sample. (It appeared that the ExpressHyb partially solubilised material in each sample (this could be amorphous carbon)). The samples were gently rocked for about 15 hours at room temperature, and then at 45° C. for 1 hour. The samples were centrifuged at 10,000 rpm for 4 minutes. The ExpressHyb was removed, and 50 μl of an ˜10 nM freshly-made mixture of DNA-modified gold nanoparticles in ExpressHyb was added to samples C and E, while a freshly-made solution of unmodified gold nanoparticles in ExpressHyb was added to samples D and F, as indicated below.

| | Au-(Gold2A-SH3′) | Au |
|---|---|---|
| SWNT-NT3′ | Sample C | sample D |
| SWNT | Sample E | Sample F |

The samples were left to sit at 45° C. for 15 min, and then were gently rocked at room temperature for 8 hours. The samples were centrifuged at 10,000 rpm for 3 minutes, and supernatants were removed. The supernatant removed from Sample C was colourless (indicating that most of the DNA-modified gold nanoparticles were bound to the nanotubes), while the supernatants from Samples D, E and F were pink (indicating that most of the gold nanoparticles had not reacted with the nanotubes).

The samples were washed three times with 80 μl of 0.1M NaCl, 10 mM phosphate buffer to remove excess ExpressHyb and unbound gold nanoparticles.

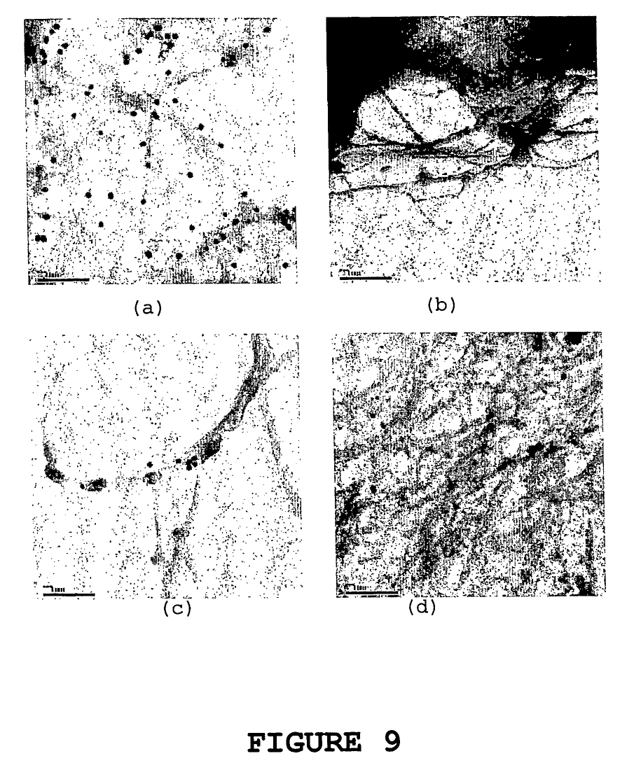

TEM images of the samples are shown in FIG. 9. The TEM images reveal that the single-walled nanotubes are not isolated, but rather form bundles of nanotubes which in turn are matted with other bundles. A large number of discrete gold nanoparticles are seen in sample C (FIG. 9(a)), while a few isolated gold nanoparticles are seen in samples E (FIG. 9(c)) and F (FIG. 9(d)). The much larger number of gold nanoparticles in Sample C, compared with the controls, indicates that DNA chemically attached to the nanotubes is binding specifically to the DNA on the gold nanoparticles. The few nanoparticles in samples E and F may be physically trapped in the nanotube mats. Since the nanotubes are bundled, it is not possible to locate precisely the tip of any single nanotube, and hence to see if the gold nanoparticles are positioned there. However, the TEM image of sample C (FIG. 9(a)) is quite different from those of samples I and J in FIGS. 8(a) and 8(c), respectively, where the DNA is attached to the surfaces of multi-walled nanotubes, and so it is likely that DNA is on the tips of the single-walled nanotubes in sample C.

Experiment F. Visual Assay to Determine if DNA Physically Attached to Nanotubes by In Situ Synthesis from an Oligothymidine Linker is Able to Hybridise Its Complementary Strand.

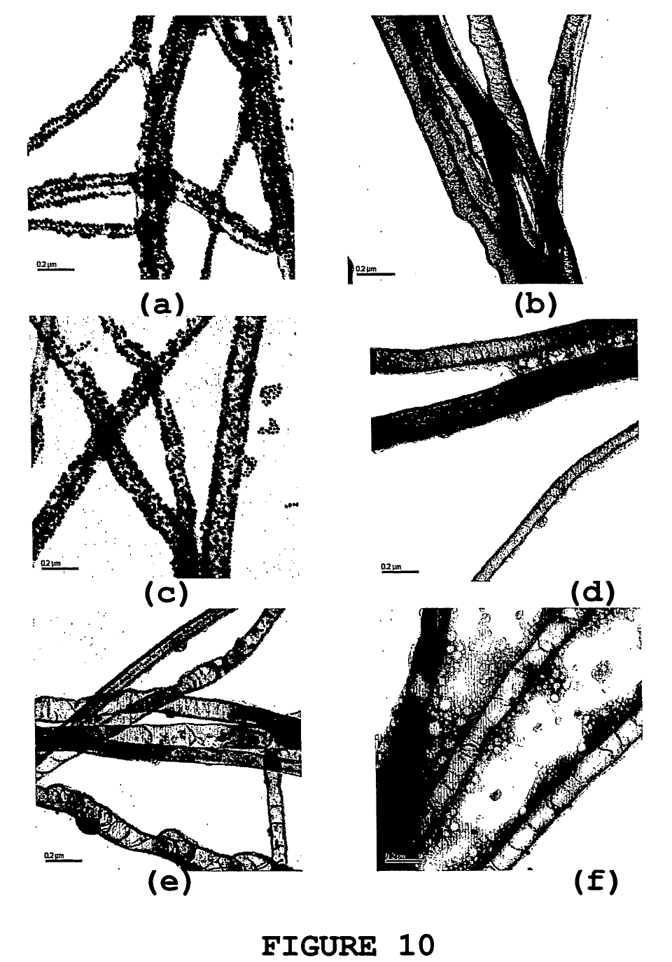

In order to check if any physically adsorbed DNA remained attached to the nanotubes (after in situ synthesis, removal of protecting groups from the bases and extensive washing), and to see if it was in a state where it could bind its complementary strand, the unmodified nanotubes that had been through in situ DNA synthesis to make NT3′T19 (Sample 1), the azidothymidine-modified nanotubes that had been through the same in situ DNA synthesis (Sample 2), and unmodified, aligned multi-walled nanotubes that had not been through the DNA synthesis (Sample 3) were assayed using the gold-nanoparticle/TEM assay as described below. Briefly, each sample was pre-incubated in three exchanges of ExpressHyb at 37° C., before a freshly-prepared solution of ExpressHyb containing gold-nanoparticles modified with DNA (Gold2A-SH3′) was added to each. For further controls, a freshly prepared solution of ExpressHyb containing gold nanoparticles (without DNA) was added to separate samples of each of the above which had also been pre-incubated in three exchanges of ExpressHyb at 37° C. All six samples were left to rock gently overnight at 37° C. Then, the ExpressHyb and unreacted gold-DNA or gold nanoparticles were removed, and the samples were washed three times with 100 μl of 0.1M NaCl, 10 mM sodium phosphate buffer, before resuspending in 50 μl 0.05M NaCl, 10 mM sodium phosphate buffer, and imaging by TEM. The TEM images are shown in FIG. 10. The similar appearance of gold nanoparticles in FIG. 10a) (sample 1, DNA physically attached to nanotubes) and 10(c) (sample 2, DNA chemically attached to nanotubes) reveals that DNA synthesized on unmodified, aligned multi-walled nanotubes is sufficiently strongly attached to the nanotubes by physical adsorption to remain in place through stringent and vigorous washings, and heating for prolonged periods at 37° C., and that this DNA is able to bind to its complementary strand on the gold nanoparticles. No gold nanoparticles appear in other controls.

It is possible that some of the thymidine nucleotides, which would form part of the T19 chain in the full-length molecule NT3′T19, may be laying against the walls of the nanotubes in Sample 1, and hence would not be available for hybridization to complementary DNA. This was not tested here, since only 16 nucleotides at the 5′ end of the DNA on the nanotubes was used for base pairing with DNA on the gold nanoparticles.

Experiment G: Binding DNA-Modified Gold Nanoparticles to DNA-Modified Mats of SWNTs.

Mats of SWNTs were functionalised with DNA molecules of different lengths (NT3′T₁₉NH₂3′ (35 nucleotides) and NT3′NH₂3′ (16 nucleotides)) by first photoetching the mats with ANB-NOS and then linking the DNA molecules with amine linkers to carboxyl groups on the succinimide moieties, as described in Example 1.4. Gold nanoparticles of 16 nm diameters were bound to DNA molecules (Gold3A-SH3′) as described above in Example 4.

Two samples of DNA-modified SWNT mats and one sample of an unmodified SWNT mat, all of approximately equal size, were placed in eppendorf tubes and incubated with ExpressHyb at 36° C. for an hour. The supernatants were removed. 50 μl of a pre-mixed solution containing equal volumes of ˜15 nM, 16 nm diameter, gold nanoparticles modified with DNA (Gold3A-SH3′) were added to each sample. The samples were gently rocked at 36° C. overnight. The samples were then centrifuged at 8000 rpm for 30 minutes, and supernatants were removed. The samples were washed three times with 80 μl of 0.1M NaCl, 10 mM sodium phosphate buffer pH 7, and stored in 50 μl of 0.05M NaCl, 10 mM sodium phosphate buffer pH 7.

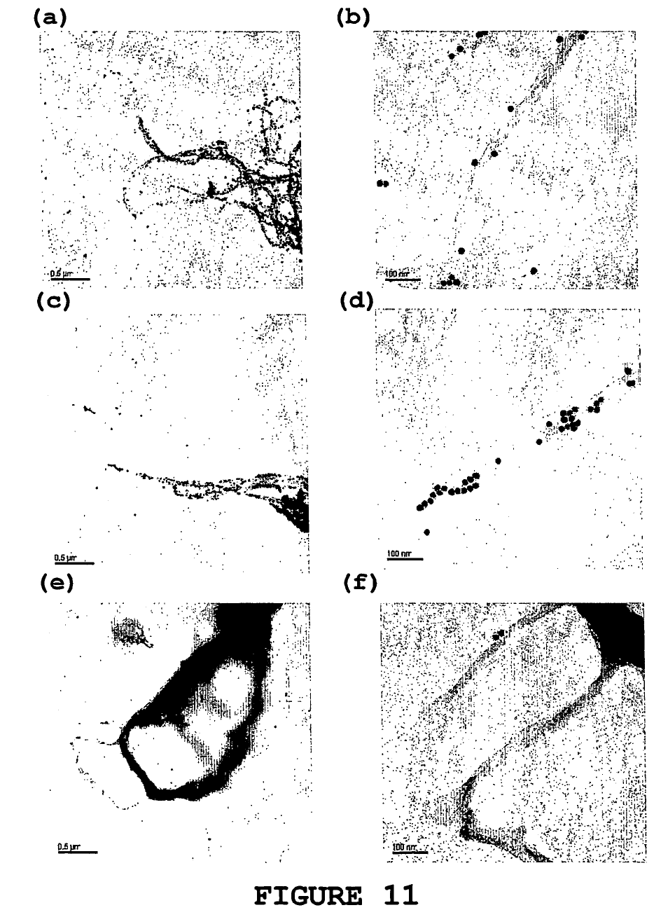

TEM images of the samples are shown in FIG. 11. FIGS. 11(a) and (b) show nanotubes taken from SWNT mats modified with DNA molecules of 35 nucleotides, while FIGS. 11(c) and (d) show nanotubes taken from SWNT mats modified with DNA molecules of 16 nucleotides. FIGS. 11(e) and (f) show nanotubes taken from mats of unmodified SWNTs. The large number of gold nanoparticles at the surfaces of the SWNTs, shown as being present in small bundles in FIG. 11(a)-(d), reveal that DNA was successfully attached to the SWNTs in mats using the ANB-NOS method, and that the sites of attachment are the walls of the SWNTs. The control (FIG. 11(e)-(f)) shows very few gold nanoparticles at the surfaces of the nanotubes, indicating that there is little non-specific physical adsorption of DNA-modified gold nanoparticles to the nanotubes, under the conditions of the experiment.

Experiment H. Checking the Attachment of Pre-Made DNA Molecules to Aligned MWNTs Using the ANB-NOS Method.

Vertically-aligned MWNTs on gold foil were functionalised with 35-nucleotide DNA molecules (NT3′T₁₉NH₂3′) by first photoetching the nanotubes with ANB-NOS and then coupling the amine groups on the linkers of the DNA molecules to carboxyl groups on the succinimide moieties, following the method described in Example 1.4. Gold nanoparticles of 16 nm diameter were bound to DNA molecules (Gold3A-SH3′) as described above in Example 4.

A sample of DNA-modified MWNTs on gold foil, and a sample of unmodified MWNTs on gold foil, of approximately equal size, were placed in eppendorf tubes and incubated with ExpressHyb at 36° C. The supernatants were removed. 50 μl of a pre-mixed solution containing equal volumes of ˜15 nM, 16 nm diameter, gold nanoparticles modified with DNA (Gold3A-SH3′) were added to each sample. The samples were gently rocked at 36° C. overnight. The supernatants were removed. The samples were washed three times with 80 μl of 0.1M NaCl, 10 mM sodium phosphate buffer, pH 7, and stored in 50 μl of 0.05M NaCl, 10 mM sodium phosphate buffer, pH 7 at 4° C.

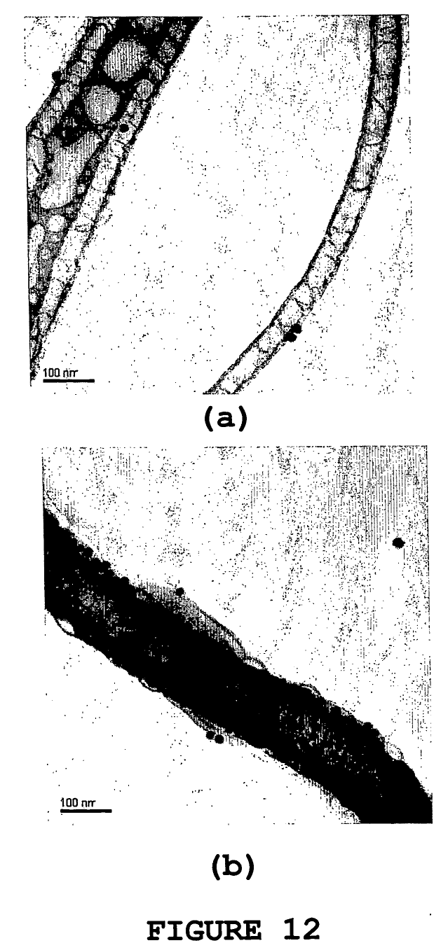

TEM images of the samples are shown in FIG. 12. FIG. 12(a) shows unmodified MWNTS, while 12(b) shows DNA-modified MWNTS. The significantly higher numbers of DNA-modified gold nanoparticles lying close to the surfaces of the MWNTs in (b), compared with (a), indicate that the DNA on the gold nanoparticles is hybridizing with DNA on the surfaces of the MWNTs, and therefore that the attachment of DNA to MWNTs by the ANB-NOS method was successful.

Experiment I. Binding DNA-Modified Gold Nanoparticles to Complementary DNA at the Tips of SWNTs, with Possible Tip-To-Tip Linkage of SWNTs Mediated by DNA-Modified Gold Nanoparticles

DNA molecules with 3′ amine linkers of sequence

5′ TACGCGAATTGCCACT-(CH₂)₇-NH₂ 3′ (NT3′NH₂3′)

were coupled to oxidised SWNTs dispersed in aqueous solution, according to the method described in Example 1.6. We used the gold nanoparticle assay to check where the DNA was attached to the SWNTs. The DNA modified SWNTs were soaked in three exchanges of 50 μl ExpressHyb for 15 minutes each. The samples were centrifuged and ExpressHyb was removed. A pre-mixed solution of 5 μl of ˜15 nM gold nanoparticles (˜16 nm diameter) modified with DNA (Gold3A-SH3′ of sequence 5′ AGTGGCAATTCGCGTATTCATCCTCAACAT-(CH₂)₃—S-[S—(CH₂)₃—OH] 3′) and 45 μl ExpressHyb was added to the DNA-modified SWNT sample. The mixture was rocked gently overnight at 34° C. The supernatant was removed, and the sample was washed three times with 100 μl 0.1M NaCl, 10 mM sodium phosphate buffer, pH 7, and then stored at 4° C. in 0.05M NaCl, 10 mM sodium phosphate buffer, pH 7.

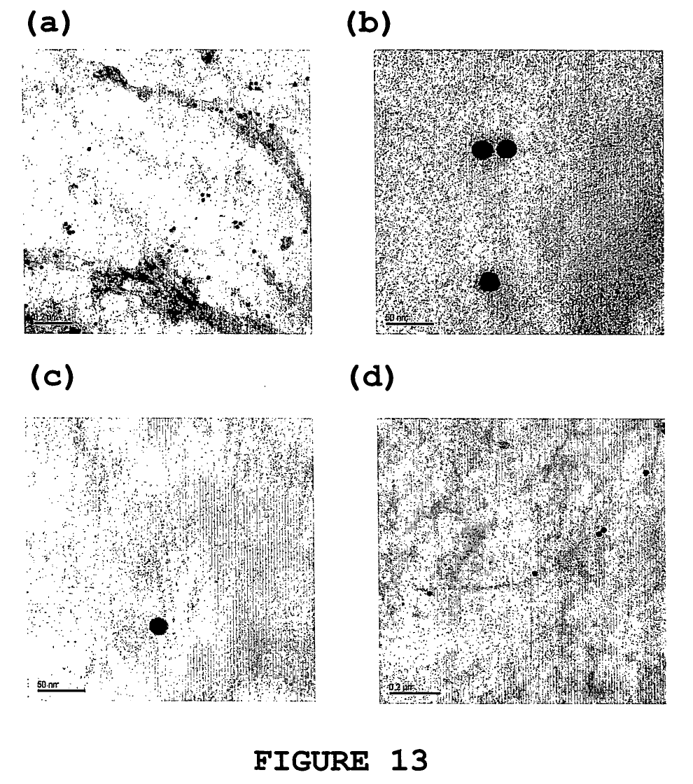

TEM images of the SWNTs are shown in FIG. 13. Generally the SWNTs are not isolated but occur in bundles of two or more nanotubes. Therefore it is difficult to see exactly where one nanotube starts and ends, and hence where the gold nanoparticles are binding on the SWNTs through DNA hybridisation. However, the TEM images for this experiment show a pattern of attachment of gold nanoparticles to the SWNTs (FIG. 13(a)) that is different from those shown for the experiment depicted in FIGS. 11(a) and (b) where the gold nanoparticles are most likely attached through DNA hybridisation to the walls of the SWNTs. Thus the sites of DNA attachment in this experiment is most likely at the tips, as indicated in FIG. 13(b) and possibly at defects in the sidewalls (FIG. 13(c)), of the SWNTs.

When a single gold nanoparticle modified with single-stranded DNA (Gold3A-SH3′) binds to the tip of a DNA-modified SWNT, there remain many single-stranded DNA molecules (Gold3A-SH3′) on the gold nanoparticle that are free to bind to complementary DNA (NT3′NH₂3′) on the tips (or at defects in the sidewalls) of other SWNTs. Such binding would result in the linking of SWNTs end-to-end (if the linking is through DNA attached on the tips) and side-by-side (if the linking is through DNA attached at wall defects). Such a possibility is shown in FIG. 13(d) where a number of nanotubes in very small bundles appear to be linked end to end by gold nanoparticles. A similar image would be obtained if the gold nanoparticles were simply bound through DNA hybridisation to the tips of several SWNTs within a bundle. It is not possible to distinguish between these two possibilities with images of SWNTs at this resolution.

Experiment J. Visual Assay to Determine the Extent of Attachment of DNA to Aligned MWNT, Down the Length of the Nanotubes, and Throughout the Sample.

The TEM images of gold nanoparticles with nanotubes shown in the above experiments contain just a few representative samples of nanotubes taken from a much larger sample. To determine how representative these images are of the larger sample, we made cross-sections of a sample of vertically-aligned, DNA-modified MWNTs on gold foil to which we had hybridised DNA-modified gold nanoparticles of 16 nm diameter.

DNA (NT3′T₁₉NH₂3′) was attached to aligned MWNTs on gold foil using the ANB-NOS method as described in Example 1.4. Gold nanoparticles of 16 nm diameter were functionalised with DNA (Gold3A-SH3′) as described above in Example 4. As described above in Experiment H, the DNA-modified MWNTs were incubated in ExpressHyb, and then DNA-modified gold nanoparticles in ExpressHyb were added and allowed to bind over a period of 24 hours, followed by multiple washing.

To make the thin cross-section from the nanotube sample, the following procedure was developed. A sample of approximately 1 mm by 3 mm was placed in a glass specimen jar. The sample was dehydrated through a series of changes of increasing ethanol concentrations (50%, 70%, 95%, 100%, 100%, 100%) for 15 minutes each. Then the sample was placed in 1:1 ratio of 100% ethanol:resin for 4 hours, followed by placement in 100% resin overnight. A rubber mould was filled with fresh resin, and the sample was placed in the mould. This was left in an oven at 37° C. for six days to cure the resin. The block was trimmed, and then ultra-thin sections were cut using a diamond knife. Sections ˜150 nm thick were best when viewed with the TEM.

The resin was made from 25 ml Epon, 15 ml araldite, 55 ml DDSA (hardener) and 1.7 ml DMP-30 (accelerator) mixed together.

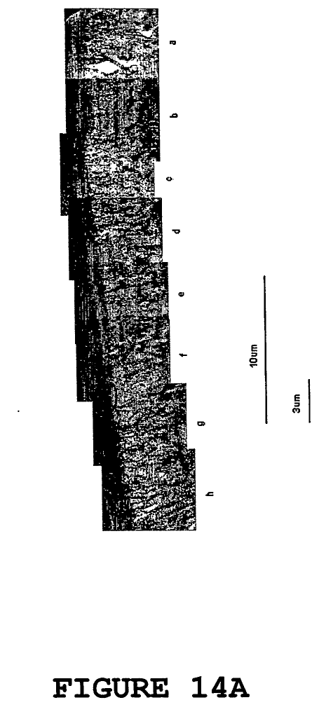

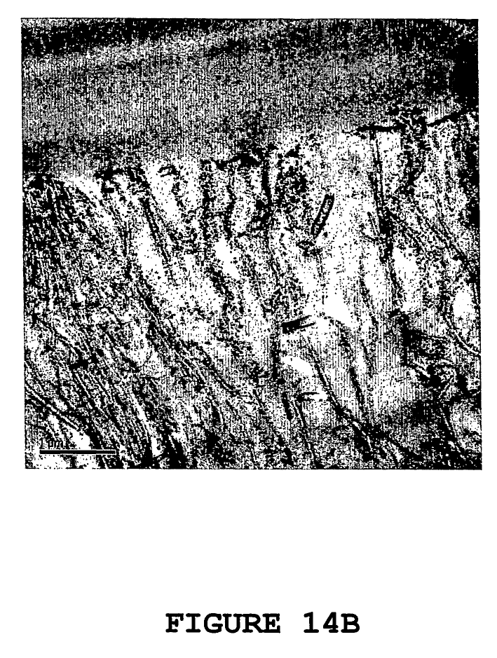

FIG. 14(A) shows overlays of consecutive TEM images taken across a thin cross-section of a sample of DNA-modified aligned MWNTs to which DNA-modified gold nanoparticles had been added. The gold nanoparticles are seen close to the surfaces of the MWNTs across the breadth of the sample. In addition, they appear close to the nanotubes all down the lengths of the nanotubes. Thus the images reveal that DNA has been successfully attached to the walls of the MWNTs extensively throughout the sample.

The gold nanoparticles seem to be present in slightly higher concentrations at the tips of the nanotubes, than on the walls. Some of the nanotubes appear broken in the image, presumably having been cut by the diamond knife when the sample was prepared. In this sample, most nanotubes have uniform diameter, although a few have much larger diameters than the average.

### EXAMPLE 5

### The Development of Self-Assembled, Nano-Scale Devices

This experiment demonstrates the use of DNA to precisely locate carbon nanotubes between two gold electrodes, and to measure the current-voltage properties of this completed, self-assembled circuit.

Aligned, multi-walled carbon nanotubes are prepared by pyrolysis of iron(II) phthalocyanine (FePc) under Ar/H₂ at 800-1100° C., checked for amorphous carbon impurity using Scanning Electron Microscopy, cleaned-up by plasma treatment, and transferred from the quartz onto a gold-foil substrate (as described by Li, D.-C., Dai, L., Huang, S., Mau, A. W. H. and Wang Z. L. (2000) Chem. Phys. Lett, 316, 349-355). Single-walled nanotubes, purchased from two commercial sources (Iljin Nanotech Co. Ltd. (Korea), and Carbon Nanotechnologies Inc. (Houston, Tex.)) are checked for purity using TEM or SEM, cleaned up, tested in various solvents and given ultrasonic treatments for various times to de-bundle and cut the nanotubes into lengths less than 1 μm. The quality of the nanotube samples so prepared are checked using SEM and/or TEM, as appropriate. The tips of the single-walled nanotubes are functionalised with carboxylic acid groups by refluxing the nanotubes in HNO₃/H₂SO₄ solution.

DNA oligonucleotides with an amino linker (NT3′-NH₂3′, and NT3′-NH₂5′), and with a thiol linker (Gold2A-SH3′) are synthesized using the Applied Biosystems DNA synthesiser. The oligonucleotides are labelled with P³² to check purity. The oligonucleotides with amino linkers are chemically attached to the sides of unmodified SWNT using the ANB-NOS method. These oligonucleotides with amino linkers are also chemically attached via an amide bond to the tips of SWNT functionalised with carboxyl groups. Oligonucleotides (NT3′-T19) are built up from azido-thymidine (previously bound by photochemical reaction to the walls of the MWNT) using the Applied Biosystems DNA synthesizer. Oligonucleotides (NT3′-T19), physically attached to the MWNT, are also built up by synthesis in the Applied Biosystems DNA synthesizer, starting with unmodified MWNT.

The MWNT and SWNT samples are analysed by XPS to determine the extent of chemical and/or physical attachment of DNA. Gold nanoparticles, of diameter approximately 15 nm are prepared as described above. The oligonucleotide with a thiol linker, Gold2A-SH3′, is bound to the gold nanoparticles as described above. The locations of the DNA molecules on the MWNT and SWNT are determined by hybridizing these DNA molecules with the complementary DNA molecules on the gold nanoparticles and visualizing the locations of the gold nanoparticles using TEM, as described above.

A gold coating is sputtered over a silicon substrate. The gold is patterned to create a simple electrode system which serves as the test system plus control. Non-specific sticking of DNA-modified carbon nanotubes to the silicon substrate is checked by imaging with AFM. Where necessary, the silicon substrate is covered with a self-assembled monolayer of molecules with exposed negatively charged functional groups in order to reduce non-specific binding of DNA-modified nanotubes.

Using a mask, single-stranded DNA molecules (pre-made, with thiol linker) are spotted on to the gold electrodes in the desired locations. The mask is removed, and the chip is dipped into MAAD (mercapto-acetic-acid-dimer) to cover remaining gold surfaces with a negatively-charged self-assembled monolayer. The carbon nanotubes are positioned across the gold electrodes using hybridization between the DNA on the modified nanotubes and their complementary DNA molecules bound to the gold electrodes. The control is unmodified nanotubes. AFM is used to image the results.

The positioning of nanotubes in a defined pattern on the gold electrodes demonstrates in principle that DNA may be used as an agent for self-assembling devices.

Current-voltage curves for the two-electrode system spanned by a nanotube or group of nanotubes are measured. The measurements are repeated for the control where two electrodes are not spanned by a nanotube.

If conductance is not sufficiently high, refinements may be made in subsequent work by improving the conductance of the DNA (for example by using metal-bound phosphorothioated DNA, or by incorporating gold nanoparticles at the junctions, or by silver-coating the DNA).

### EXAMPLE 6

### The Development of Self-Assembled Nanoscale Electronic Devices, Using DNA-Directed Assembly

This Example describes progress made towards positioning a DNA-modified nanotube across two gold electrodes with the purpose of assembling a nanoscale circuit or device. Nanotubes hold great promise as components in nanoscale electronic devices because of their great mechanical strength, their high thermal conductivity, and their electronic properties. Nanotubes may show metallic or semi-conducting properties.

(6.1) Fabrication of Electrodes.

Since the nanotube is required to span two electrodes, the distance between the electrodes must be compatible with the length of the nanotube. This distance may be of the order of 10 nm to several microns.

The electrodes were made on a Au/Nb bilayer thin film by standard photolithography processes in a clean room. The processes mainly consisted of two parts: thin film deposition, and electrode patterning.

Thin film deposition. A single crystal MgO (001) substrate of area 10 mm×10 mm was used to deposit the Au/Nb thin film. The substrate had a roughness of less than 2 nm. The Au/Nb bilayer thin film was deposited on the MgO surface using a DC magnetron sputtering technique. The Nb film was first deposited and then the Au film was deposited without breaking the vacuum. The thickness of the films was controlled by the working pressure, time of deposition and the sputtering power. The thicknesses of the Nb and Au films are 20 and 25 nm respectively.

(6.2) Electrode Patterning.

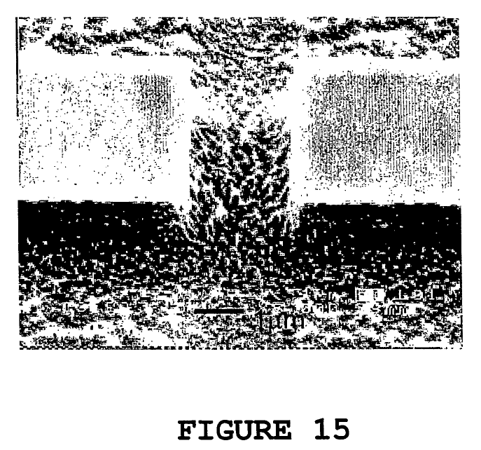

The bilayer thin film was patterned into electrodes in the form of a microbridge with a gap between the two ends of the electrode. The patterning of this structure was done by standard photolithography processes. Briefly, a positive resist layer was spun on top of the film. A chromium mask with the desired pattern was used to transfer its pattern into the photoresist using UV exposure and chemical development. The photoresist mask was then used as a mask to transfer the pattern into the films by ion beam etching. Energetic Argon ions were used to etch away the unwanted parts of the films, leaving the electrode parts protected by the photoresist. After the etching process, the photoresist was stripped off chemically to complete the electrode patterning process. A scanning electron microcroscopy (SEM) image of the gap between the gold electrodes on Nb film is shown in FIG. 15.

(6.3). Binding DNA to Gold Electrodes.

Conditions for binding functional DNA molecules to gold surfaces through disulphide linkers were developed using gold-coated glass slides. In a typical experiment, 1 nL to 1 μl drops of DNA of base sequence

5′ AGTGGCAATTCGCGTA-(CH₂)₃-S-[S-(CH₂)₃-OH] 3′
(Gold2A-SH3′, 16 nucleotides)
or
5′ AGTGGCAATTCGCGTATTCATCCTCAACAT-(CH₂)₃-S-[S-(CH₂)₃-OH] 3′

(Gold3A-SH3′, 30 nucleotides) in binding buffer were placed on gold-coated glass slides freshly cleaned in piranha solution, washed well with water, and air-dried. The slide was placed for a set time in a sealed plastic dish with a humid environment (water-soaked filter paper) to prevent the drop of DNA from drying out. The drop of DNA was washed well with water to remove unbound DNA. The slide was then placed into a solution of 6-mercapto-1-hexanol (Aldrich) in ethanol to allow the mercaptohexanol to bind to the gold surface free of DNA and thus limit non-specific binding of DNA molecules with complementary base sequence to these places. The slide was washed first with ethanol and then water to remove excess mercaptohexanol.

To find the conditions for binding DNA to gold surfaces which produced optimal binding of complementary DNA (to be attached to nanotubes), free ³²P-labelled DNA was added to the DNA bound to the gold surfaces, and the extent of hybridization or non-specific binding was monitored by quantifying the radioactivity. To do this, the slide was placed in a well, and covered with a solution of 1 μM DNA (spiked with radioactively-labeled DNA of the same sequence) in 0.89M NaCl, 10 mM Tris, pH 7.6, 1 mM EDTA, and left to gently rock for 1-2 hours. The DNA in this solution was labeled on its 5′ end with ³²P, as described in Example 4 above. The ³²P-labeled DNA had either the same base sequence (5′ AGTGGCAATTCGCGTA 3′, NT3′AS) as the DNA bound to the gold, or the complementary base sequence (5′ TACGCGAATTGCCACT, NT3′). ³²P-labeled NT3′AS tested for non-specific binding of DNA to the gold-bound DNA, while ³²P-labeled NT3′ tested for specific binding. Quantification of the radioactive counts, remaining on the gold surface after washing, permitted optimization of several parameters for maximum retention of the complementary DNA on the DNA bound to the gold surface. Radioactive counts on the samples were quantified by exposing the samples after washing and drying to a Phosphor screen, and scanning and quantifying on a Typhoon 8600 Variable Mode Imager (Molecular Dynamics) using Image Quant software and Storage Phosphor mode.

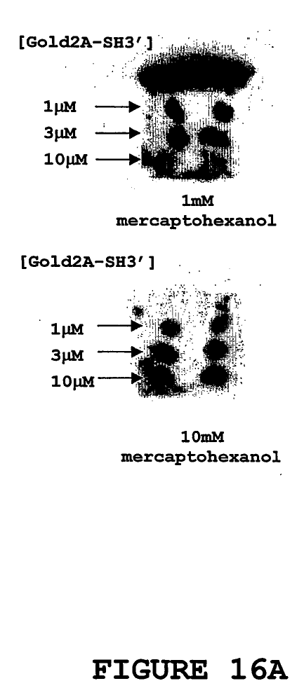

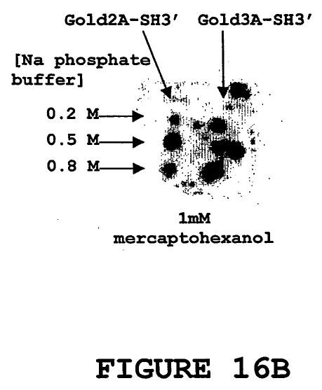

Various parameters in the above procedure were investigated, some of which are shown in FIGS. 16A & 16B. The optimal conditions are as follows. Optimal gold-binding buffer: 1 μM DNA (with disulphide linker) in 0.5M sodium phosphate buffer, pH 7. Time for binding DNA to gold surface: 30 minutes. Time for binding 1 mM mercaptohexanol in ethanol to gold surface: 30 minutes. The conditions for binding the shorter DNA molecule (Gold2A-SH3′, 16 nucleotides), which provide optimal hybridizing conditions for the ³²P-labelled DNA of complementary sequence, are more restricted than for immobilizing the longer DNA molecule (Gold3A-SH3′, 30 nucleotides), as shown in FIG. 16(B).

(6.4). Preparation of DNA-Modified MWNTs and SWNTs.

Since the nanotube is required to span the two electrodes, the length of the nanotube must be compatible with the distance between the electrodes. The lengths of nanotubes in a heterogeneous sample may vary considerably. Therefore, it is advantageous to use nanotubes with DNA attached to their walls, rather than their tips, in order to more readily locate the nanotubes exactly where required on the electrodes. This reduces the requirements for making nanotubes of precisely controlled lengths to match the spacing between complementary DNA spots on the two electrodes. In addition, the surface coating of the negatively-charged DNA molecules on the walls of the nanotubes acts as a surfactant to keep the nanotubes dispersed in the aqueous solutions required for DNA hybridisation.

MWNTs with DNA coating their walls were made by the method described in Example 1.4. The MWNTs were released from the gold foil by sonicating from 5-30 minutes in water, or in a solution of 0.1M NaCl, 10 mM sodium phosphate pH 7. The larger the diameter of the MWNTs, the longer must be the DNA attached to the surfaces of the MWNTs for the DNA to be effective in assembling the MWNT on the electrodes.

SWNTs with DNA coating their walls were made by the methods described in Example 1.8.

MWNTs and SWNTs were made with DNA attached to their tips by the methods described in Examples 1.2a and 1.2b and 1.6.

(6.5). Measuring the Current-Voltage Curve by Scanning Tunnelling Spectroscopy from a DNA-Modified MWNT Lying on a Gold Surface.

Nanotube samples suitable for STM (Scanning Tunnelling Microscopy) studies were prepared by spin coating nanotube suspensions on to Au(111) surfaces. Unmodified MWNT suspensions were in methanol, and DNA-modified MWNT suspensions were in MilliQ water. The DNA attached to the walls of the MWNTs by the ANB-NOS method described in Example 1.4 was NT3′NH₂3′ with base sequence 5′ TACGCGAATTGCCACT-(CH₂)₇—NH₂ 3′.

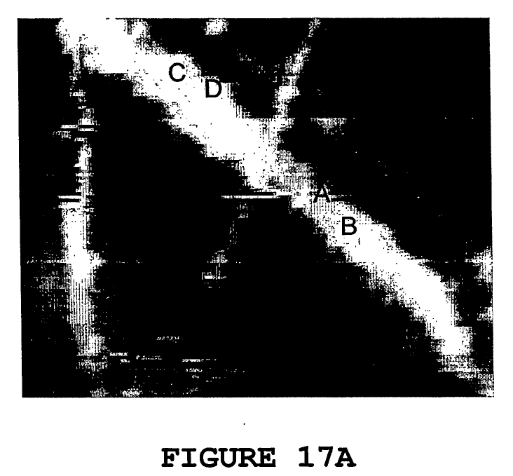

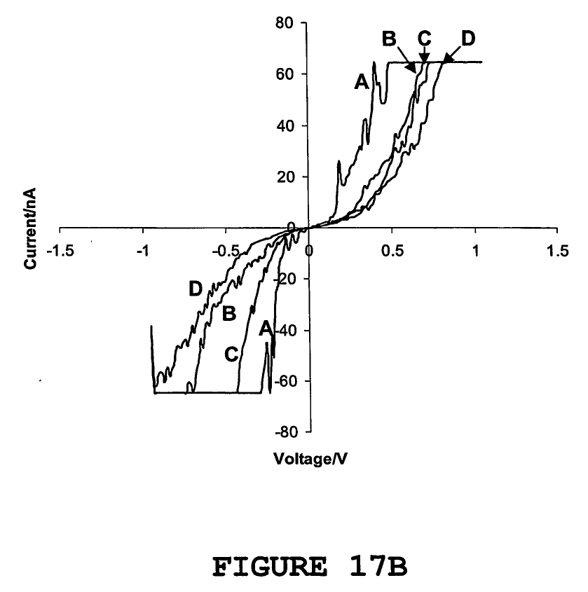

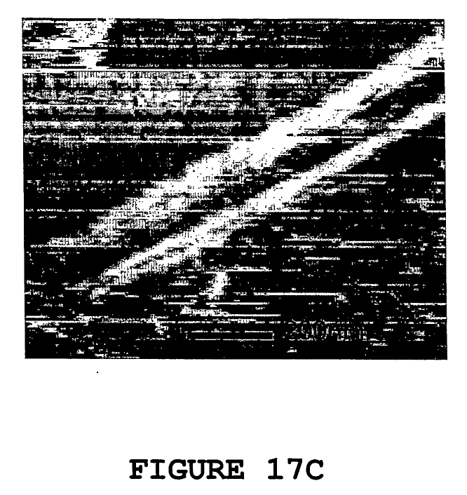

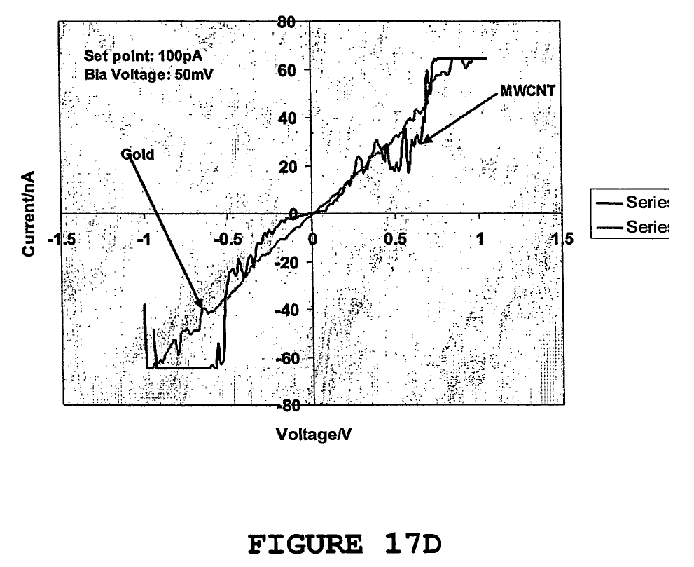

STM imaging studies were performed with a Topometric Discover 2010 STM using Pt/Ir tips in constant current mode with the bias voltage (V) applied to the tip. The resolution and calibration of the STM were confirmed in situ by imaging the atomic lattice and steps of the Au(111) substrate surface. Imaging set points were typically 50 mV at 100 pA. A STM image of some DNA-modified MWNTs on a Au(111) surface is shown in FIG. 17(a). A STM image of unmodified MWNTs on a Au(111) surface is shown in FIG. 17(c).

STS (Scanning Tunnelling Spectroscopy) measurements were made by averaging 5-10 current versus voltage (I-V) curves at specified locations on individual MWNTs. The precise nature of the electrical contacts between the STM tip, the MWNT and the gold surface are experimentally difficult to determine. To ensure the reliability of these measurements, we routinely checked that clean areas of the Au(111) substrate exhibited characteristic I-V curves. The I-V measurements for a DNA-modified MWNT were performed at four positions indicated by the symbols A, B, C and D on the MWNT in FIG. 17(a), and their corresponding I-V curves are displayed in FIG. 17(b). Each of these I-V curves was reproducible for a given tip position. The semi-conducting current-voltage characteristics observed for the DNA-modified MWNT (FIG. 17(b)) are similar to those measured for an unmodified MWNT (FIG. 17(d)), indicating that DNA attached to the surface of the nanotube has little effect on the electronic properties measured in this experiment.

(6.6). Measuring the Current-Voltage Curve Down the Length of a DNA-Modified MWNT by STS.

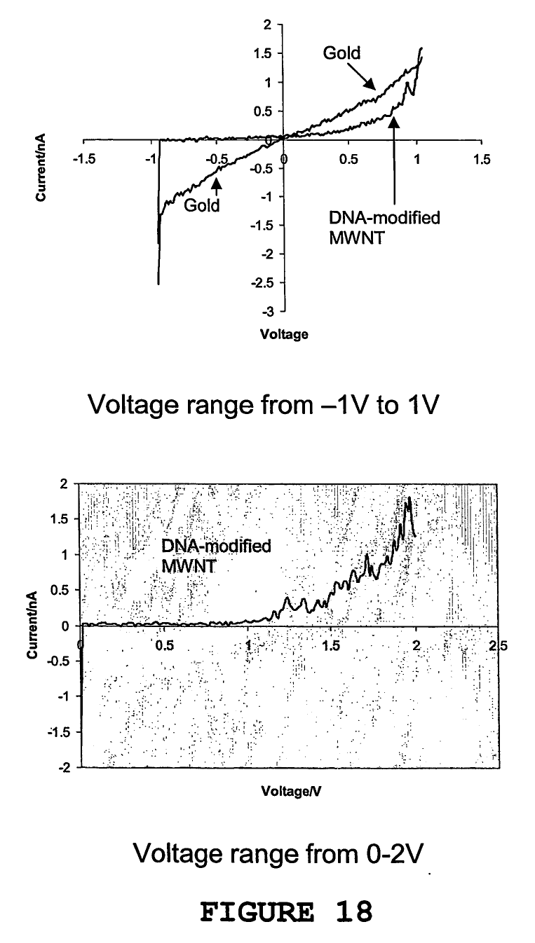

These experiments were performed in a similar manner to those described in 6.5 above, except that in this case the STM tip was driven forward into the nanotubes lying on the flat gold surface until a metallic contact was formed. Upon retraction of the tip beyond the normal tunnelling range, continuous electrical contact indicated that a nanotube now spanned the gap between tip and the surface, and hence current-voltage measurements could be made down the length of the nanotube. The current-voltage curves obtained are shown in FIG. 18. The two DNA-modified MWNTs which were investigated have the properties of a semi-conductor.

(6.7). DNA-Directed Placement of DNA-Modified Nanotubes Between Two Gold Electrodes.

Gold electrodes are made as described above. As described in 6.3, DNA molecules with disulphide or thiol linkers are bound to the ends of two freshly-cleaned gold electrodes separated by less than the length of the DNA-modified nanotubes. The drops of DNA may be placed accurately at the electrode ends using glass pipettes pulled finely to deliver nanolitre droplets, by modified inkjet printing, by dip-pen lithography (modified AFM tips), or by masks. DNA-modified SWNTs or MWNTs are prepared as described in above. The DNA attached to the nanotubes has a base sequence which is complementary to all or part of the DNA bound to the gold electrodes. A drop of DNA-modified nanotubes (either SWNT or MWNT) dispersed in a solution of between 0.1 and 0.5M NaCl, 10 mM sodium phosphate buffer, pH 7, is placed over the gap between two gold electrodes and the complementary DNA bound to the gold electrodes. The system is gently rocked for several hours to allow hybridisation to occur between the DNA on the nanotubes and the complementary DNA on the electrodes. This results in nanotubes being placed between the electrodes. Current-voltage curves may be measured by connecting the other ends of the gold electrodes in a standard circuit. The semi-conducting properties of nanotubes assembled into such a device would make the devices useful as field effect transistors, and DNA sensors.

### EXAMPLE 7

### DNA Sensor

(a) DNA Sensor (Electrochemical Sensor)

The procedure to assemble an electrochemical sensor is described below. This procedure involves the use of an electroactive indicator, which intercalates the double stranded DNA. Transition metal complexes such as Co and Ni complexes, or reagents such as Daunomycin hydrochloride (Marrazza G. et al., 1999, Biosensors & Bioelectronics 43-51) can be used as intercalators. The immobilized DNA on the electrode, which can be made from aligned MWNTs or mats of SWNTs, is incubated in the sample solution containing the target DNA and the electroactive indicator for 10 min and then washed with buffer solution using 0.02 M phosphate buffer pH 7.0, with 0.05 M NaCl. The potential value of the anodic peak for the transition metal complex or daunomycin obtained in differential pulse voltammetry is used to detect the presence and the amount of the complementary DNA. Alternatively, the hybridization process is monitored with a chronopotentiometric transduction mode.

(b) DNA Sensor (Optical Sensor) Based on Surface Plasmon Resonance (SPR)

Surface plasmon Resonance (SPR) biosensors can be generally constructed as miniature integrated sensors. (Furlong E. et. al. (1998). Biosensors and Bioelectronics 13, 1117-1126). Construction of a SPR-based DNA sensor which is portable and inexpensive is described below.

The DNA modified aligned MWNTS on gold foil is introduced into the sensor chamber and the entire structure is encased in clear epoxy. The flow cell restricts the delivery of the reagents and analyte to the region of the sensor surface producing the SPR signal. The sensor contains a light emitting diode, a P-polariser, a thermistor and a linear photodiode detector array. The change in refractive index of the surface is monitored over time as either the analyte DNA or analyte DNA and oligonucleotide probe, which is attached to a fluorophore or colloidal gold, hybridizes to the DNA probe bound to the nanotubes.

It is well known that the refractive index is proportional to the density of the molecules captured on the surface, either adsorbed or attached, and therefore the change in the refractive index measures specific binding of the analyte to the capture molecule. Therefore the use of high surface density material such as aligned MWNTs will have advantages over flat surfaces such as gold. Also, the specificity and sensitivity of this device is enhanced when an additional DNA probe, attached to colloidal particles, binds the captured analyte. The large increase in the surface density causes greater changes in refractive index.

(c) DNA Sensor Based on a Piezoelectric Transducer.

DNA-modified MWNTs on gold foil can be attached to the crystal surface of a Quartz crystal microbalance. A change in mass can be detected when the analyte DNA binds the probe DNA attached to the nanotubes. The change in mass can be enhanced by hybridizing a second DNA probe, bound to a colloidal particle, to the captured analyte DNA. The basics of the QCM is reviewed in detail by Collings et. al 1997, Rep. Prog. Phys., 60, 1397-1445.

(d) DNA Sensor Based on a Superconducting Quantum Interference Device (SQUID) Magnetometer

In this sensor, the target DNA is hybridized to DNA probes attached to aligned MWNTs on gold foil, or to SWNT mats, placed in a cell. After washing, a second oligonucleotide probe, attached to a magnetic bead, is added and hybridized to the captured target DNA. After stringent washing to remove excess DNA-modified magnetic beads, the cell is swiped through the magnetic field of a SQUID. The degree of hybridization is measured by the SQUID detecting the change in magnetic field produced by the captured magnetic beads.

(e) DNA Array

Vertically aligned MWNTs, or patterned and aligned MWNTs, on gold foil are placed on a support. The nanotubes are photoetched with a nucleotide that has a photocleavable blocking group. A mask is placed accurately on the array and the photocleavable protecting groups are removed from the desired locations by photoirradiation. Presynthesised DNA can then be coupled to the exposed nucleotides. The process is repeated for attaching DNA molecules of other sequences to other defined locations. Alternatively, nucleotides can be added step by step to the deprotected nucleotides in desired locations by repeated use of photo-irradiation and masking steps. By this process DNA molecules of desired sequence are built in situ in desired locations.

Analyte DNA is added to the array, and is detected by any of the methods described above for a DNA sensor. These methods of detection are in addition to the methods widely used, which includes detection of fluorescent molecules.

Conventional DNA array technology can only immobilize one DNA molecule per surface site. The use of vertically aligned MWNTs for a DNA array would result in a single site on the array surface effectively immobilizing hundreds of thousands of DNA molecules. In addition, the erect nature of the nanotubes ensures that the immobilized DNA is held off the array surface to create a three dimensional array, and thus the DNA is also highly available for hybridization. This combination of high loading and high availability of DNA molecules on the surface of individual nanotubes means that very few nanotubes would suffice for each DNA sequence and therefore the DNA array could be miniaturised to the extreme.

### EXAMPLE 8

### DNA-Directed Assembly of Multiple Monolayers of Nanoparticles of Different Sizes and/or Composition on the Surfaces of Carbon Nanotubes, for Changing the Surface Properties of the Nanotubes

Our visual assay using DNA-modified gold nanoparticles and Transmission Electron Microscopy reveals where functional DNA has been attached to the nanotubes. In this assay, single strands of DNA (of base sequence X) are first bound to the surfaces of gold nanoparticles, and are then hybridized to the single strands of DNA (of base sequence Y) attached to the nanotubes. For hybridization to occur, all or part of X must have a base sequence complementary to that of all or part of Y. This results in gold nanoparticles covering the surfaces of the nanotubes, in the experiments where we have attached strands of DNA all over the surfaces of the nanotubes. The coverage of gold nanoparticles on the surfaces of the nanotubes can affect several surface properties, such as refractive index, reflectivity, and surface plasmon resonance.

The surface properties of the nanotubes may be altered further by binding more DNA-modified nanoparticles of different size or different composition to the remaining free single strands of DNA on the nanoparticles covering the surfaces of the nanotubes. Here, the base sequence of the DNA strands on the nanoparticles of different size or composition is Z, where all or part of Z has the base sequence complementary to all or part of X, and Z may have the same base sequence as Y.

In this manner, several layers of DNA-modified nanoparticles of different size, shape or composition may be added sequentially to the walls of the nanotubes, to finely tune the surface properties of the nanotubes for any desired purpose. The structure of the multiple layers may be controlled by the surface coverage of the DNA attached to the walls of the nanotubes. If the DNA is attached over the surface of the nanotubes at high density, the first and subsequent layers of nanoparticles will be attached uniformly to provide an even coverage. If the DNA is attached with low density of coverage over the nanotubes, the first layer of nanoparticles will bind with low packing density to the nanotubes, and nanoparticles added subsequently will bind in clumps centred around these sites.

Two solutions of gold nanoparticles, of diameters 16 nm and 38 nm, were made according to the method described by Frens (1973) (Frens, G. Nature 241, 20-22 (1973) “Controlled nucleation for the regulation of the particle size in monodisperse gold suspensions”.) The diameters of the gold nanoparticles were measured using TEM. DNA molecules (Gold3A-SH3′) of sequence

5′ AGTGGCAATTCGCGTATTCATCCTCAACAT-(CH₂)₃-S-[S-CH₂)₃-OH] 3′

were bound to gold nanoparticles of 16 nm diameter as described earlier in Example 4. DNA molecules (Gold2B-SH3′) of base sequence

5′ TACGCGAATTGCCACT-(CH₂)₃-S-[S-(CH₂)₃-OH] 3′

were bound to gold nanoparticles of 38 nm diameter. These DNA-modified gold nanoparticles were made by mixing 1 ml of 4.2 nM gold nanoparticles of 38 nm diameter with 35.8 μl of 279 μM Gold2B-SH3′. After 24 hours, 100 μl 1M NaCl and 10 μl 1M sodium phosphate buffer pH 7 was added, and the solution was rocked gently for 48 hours. The mixture was centrifuged at 10,000 rpm for 30 minutes and the supernatant was removed. The gold nanoparticles were washed with 500 μl 0.1M NaCl, 10 mM sodium phosphate buffer pH 7, the sample was centrifuged, and the supernatant removed. The gold nanoparticles were dispersed in 310 μl 0.1M NaCl, 10 mM sodium phosphate buffer pH 7, to form a grape-coloured solution with concentration ˜13 nM.

DNA molecules (NT3′T₁₉NH₂3′) with amine linkers at the 3′ ends were attached to the surfaces of vertically-aligned MWNTs photoetched with ANB-NOS, as described in Example 1(4). (The same method, as described in Example 1(4), can be used to attach DNA with either a 3′ or 5′ amine linker to nanotubes photoetched with ANB-NOS.) The base sequence of the oligonucleotide attached to the MWNTs was

5′ TACGCGAATTGCCAC(T)₁₉-(CH₂)₇-NH₂ 3′.

The DNA-modified, vertically-aligned MWNTs on gold foil were briefly sonicated to detach them from the foil. The nanotubes dispersed well in water. A mixture of 40 μL of the DNA-modified MWNT solution and 50 μl ExpressHyb was rocked gently at 30° C. for 30 minutes. The mixture was centrifuged for 15 minutes, the supernatant was removed, and the DNA-modified MWNTs were re-dispersed in 50 μl ExpressHyb. After gently rocking for a further 30 minutes, the sample was again centrifuged for 15 minutes, the supernatant was removed, and the DNA-modified MWNTs were dispersed in a pre-mixed solution containing 30 μl ExpressHyb and 20 μl of ˜15 nM gold nanoparticles, of diameter 16 nm, which had been previously bound to Gold3A-SH3′. The mixture of DNA-modified MWNTs with DNA-modified gold nanoparticles was rocked gently overnight at 30° C.

Next day, the sample was centrifuged for 10 minutes, the supernatant was removed, and the sample was washed twice with 50 μl ExpressHyb to remove excess DNA-modified gold nanoparticles of 16 nm diameter. The sample was then dispersed in a pre-mixed solution containing 20 μl of ˜13 nM gold nanoparticles, of diameter 38 nm, which had been previously bound to Gold2B-SH3′.

The mixture of 38 nm DNA-modified gold nanoparticles with 16 nm DNA-modified gold nanoparticles bound to DNA-modified MWNTs was rocked gently at 34° C. for 6 hours, then centrifuged, and the supernatant was removed. The sample was washed four times with 100 μl 0.1M NaCl, 10 mM sodium phosphate buffer pH 7 to remove ExpressHyb and excess DNA-modified gold nanoparticles of 38 nm diameter. The sample was stored in 50 μl 0.05M NaCl, 10 mM sodium phosphate buffer pH 7.

The sample was visualized by TEM by placing 1.5 μl of solution containing the sample on a 200-mesh copper grid prepared with carbon film and left to air dry. The grid was placed in the TEM (Philips CM100) and digital images were taken with a Gatan Dual Vision camera.

Note that the 16 nucleotides at the 5′ end of Gold3A-SH3′, attached through the 3′ ends to gold nanoparticles of 16 nm diameter, can hybridise with the 16 nucleotides at the 5′ end of NT3′T₁₉NH₂3′, attached through their 3′ ends to the MWNTs. The 16 nucleotides at the 5′ end of Gold2B, attached through the 3′ ends to gold nanoparticles of 38 nm diameter, can hybridise with the 16 nucleotides at the 5′ end of Gold3A-SH3′ bound to the gold nanoparticles of 16 nm diameter, and, through this hybridization, the larger gold nanoparticles of 38 nm diameter can bind to the smaller gold nanoparticles of 16 nm diameter which coat the surface of the MWNTs. The DNA molecule bound to the larger gold nanoparticles cannot hybridise directly with the DNA attached to the nanotubes, since the base sequences are not compatible.

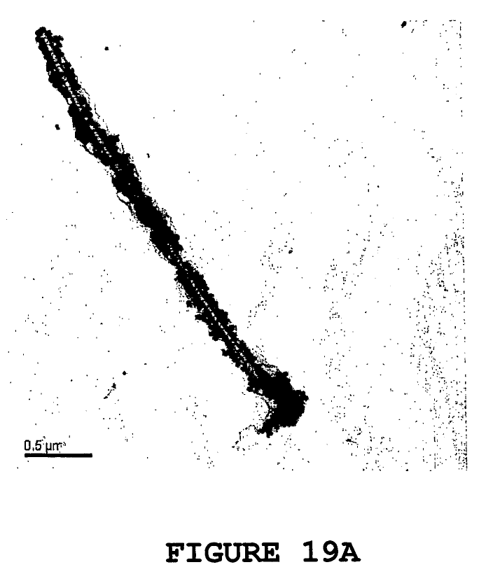

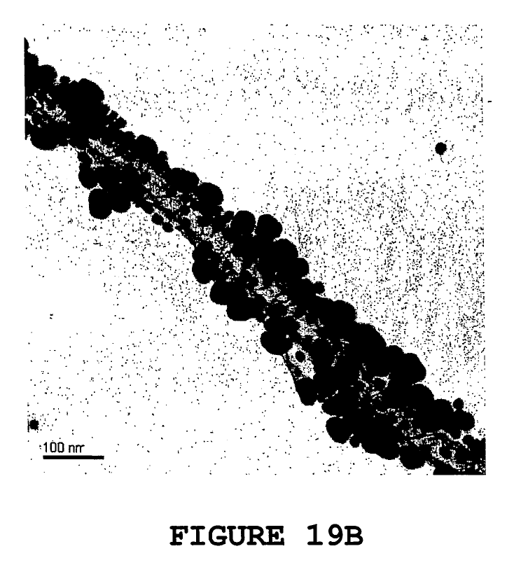

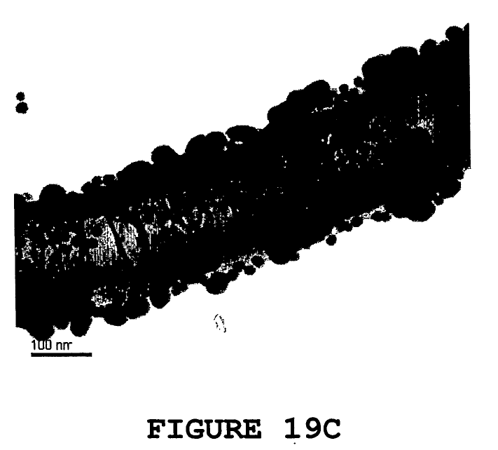

Images of MWNTs, coated with a layer of smaller gold nanoparticles (16 nm) which are in turn coated with a layer of larger gold nanoparticles (38 nm), are shown in FIGS. 19A, B & C.

### EXAMPLE 9

### Determining the Extent of Specific and Non-Specific Binding of 5′-End ³²P-Labelled DNA to Mats of DNA-Modified SWNTS, where the SWNT Mats had been Photo-Etched with Azidosuccinimide to which Pre-Made DNA was Subsequently Coupled

SWNTs in a mat, formed by filtering unmodified SWNTs dispersed in DMF, were photo-etched with ANB-NOS, and coupled to DNA molecules (NT3′NH₂3′) of base sequence

5′ TACGCGAATTGCCACT-(CH₂)₇-NH₂ 3′

following the method described in Example 1.4. The sample was washed well with several changes of MilliQ water to remove any unreacted NT3′NH₂₃′. The sample was cut with a scalpel into two pieces. One piece (a) had an area of approximately 8 mm², while the second piece (b) had an approximate area of 10 mm². Each piece was placed in a separate eppendorf tube, and 50 μl of a 50% mix of ExpressHyb in water was added to each. After rocking gently for 30 minutes at 34° C., the ExpressHyb mixture was replaced twice by 50 μl of 90% ExpressHyb in water with rocking for 30 minutes. The ExpressHyb solution was removed.

To sample (a) was added 60 μl of 0.1 μM ³²P-labelled DNA (NT3′) in 90% ExpressHyb, and to sample (b) was added 60 μl of 0.1 μM ³²P-labelled DNA (NT3′AS) in 90% ExpressHyb. The base sequence of NT3′ is

5′ TACGCGAATTGCCACT 3′,

which is identical to the base sequence of NT3′NH₂3′ which is attached to the nanotubes, and so sample (a) is a control to check for non-specific binding. The base sequence of NT3′AS is 5′AGTGGCAATTCGCGTA which is complementary to that of the DNA attached to the nanotubes, and so sample (b) determines if the DNA attached to the nanotubes is functional.

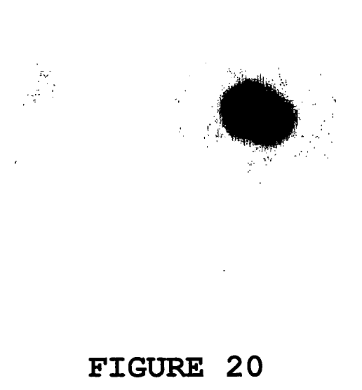

The samples were gently rocked at 34° C. for 5 hours, after which the supernatant was removed, and the samples were washed several times with 70 μl 2×SSC, 0.1% SDS, until the supernatants contained no radioactivity. The samples were transferred to a glass plate, covered with plastic film, exposed to a PhosphorImager screen, and imaged on a Typhoon 8600 Variable Mode Imager (Molecular Dynamics) using Storage Phosphor mode and Image Quant software. The images are shown in FIG. 20, where sample (a) is on the left and sample (b) is on the right. The amount of radioactivity on sample (a) is much less than on sample (b), indicating that the level of non-specific binding of ³²P-labelled non-complementary DNA to mats of SWNTs functionalised with DNA is very low compared with the level of specific hybridisation between ³²P-labelled complementary DNA and DNA attached to the SWNT mats.

These results comparing the specific and non-specific binding of DNA show that DNA-modified SWNT mats can serve as suitable substrates for DNA sensors and DNA arrays.

1. A nanotube with one or more nucleic acid molecule(s) attached thereto.

2. A method of chemically modifying a nanotube comprising either:
(i) a) chemically attaching at least one linker attached to one or more nucleic acid molecules to an optionally functionalised nanotube, wherein said linker consists wholly or partly of a functional group with the proviso that when the nanotube is functionalised with CO₂H, then the linker is not a primary aliphatic alkyl amine; and
b) synthesising at least two nucleic acid molecules, by sequential addition of nucleotides in situ, starting from said one or more nucleic acid molecules; or
(ii) a) chemically attaching at least one linker attached to one or more nucleic acid molecule to an optionally functionalised nanotube, wherein said linker consists wholly or partly of a functional group; and
b) synthesising at least two nucleic acid molecules, by sequential addition of nucleotides in situ, starting from said one or more nucleic acid molecules; or
(iii) a) chemically attaching at least one linker to an optionally functionalised nanotube, wherein said linker consists wholly or partly of a functional group; and
b) attaching one or more nucleic acid molecules to said optionally functionalised nanotube via said functional group on said linker; or
(iv) a) chemically attaching at least one linker to an optionally functionalised nanotube, wherein said linker consists wholly or partly of a functional group; and
b) synthesising one or more nucleic acid molecules, by sequential addition of nucleotides in situ, starting from said functional group on said linker

3. A method of chemically modifying a nanotube comprising either:
(i) a) photochemically attaching at least one linker attached to one or more nucleic acid molecules to an optionally functionalised nanotube, wherein said linker consists wholly or partly of a functional group; and
(b) synthesising at least two nucleic acid molecules by sequential addition of nucleotides in situ, starting from said one or more nucleic acid molecules; or
(ii) a) photochemically attaching at least one linker to an optionally functionalised nanotube, wherein the linker consists wholly or partly of a functional group; and
b) attaching one or more nucleic acid molecules to said optionally functionalised nanotube via said functional group on said linker; or
(iii) a) photochemically attaching at least one linker to an optionally functionalised nanotube, wherein the linker consists wholly or partly of a functional group; and
b) synthesising one or more nucleic acid molecules, by sequential addition of nucleotides in situ, starting from said functional group on said linker.

4. A method of physically modifying a nanotube comprising either:
(i) (a) physically adsorbing at least one anchor attached to one or more nucleic acid molecules to the surface of an optionally functionalised nanotube, wherein said anchor consists wholly or partly of a functional group;
b) synthesising at least two nucleic acid molecules by sequential addition of nucleotides in situ, starting from said functional group on said anchor; or
(ii) a) physically adsorbing at least one anchor to the surface of an optionally functionalised nanotube, wherein said anchor consists wholly or partly of a functional group; and
b) chemically attaching one or more nucleic acid molecules to said functional group on said anchor adsorbed on the optionally functionalised nanotube; or
(iii) a) physically adsorbing at least one anchor attached to one or more nucleic acid molecules to the surface of an optionally functionalised nanotube, wherein said anchor consists wholly or partly of a functional group;
b) synthesising one or more nucleic acid molecules, by sequential addition of nucleotides in situ, starting from said functional group on said anchor.

5. A method of linking nanotubes comprising the steps of:
a) attaching a first nucleic acid molecule of a first base sequence to a first optionally functionalised nanotube; and
b) hybridising the first nucleic acid molecule with a second nucleic acid molecule of a second base sequence attached on a second optionally functionalised nanotube, wherein the base sequence of the second nucleic acid molecule is substantially complementary to the base sequence of the first nucleic acid molecule.

6. A method of linking nanotubes comprising the steps of:
a) attaching a first nucleic acid molecule of a first base sequence to optionally functionalised nanotubes; and
b) hybridising the first nucleic acid molecule with a second nucleic acid molecule which comprises a base sequence substantially complementary to the first base sequence and further comprises a second or a third base sequence which is/are not complementary to the first base sequence, but is/are complementary to each other.

7. A method of linking nanotubes comprising the steps of:
a) attaching a first nucleic acid molecule of a first base sequence to a first optionally functionalised nanotube;
b) attaching a second nucleic acid molecule of a second base sequence to a second optionally functionalised nanotube;
c) hybridising the first nucleic acid molecule to a third nucleic acid molecule which comprises a base sequence substantially complementary to the base sequence of the first nucleic acid molecule and which further comprises at least 5 nucleotides which are not complementary to the base sequence of the first or second nucleic acid molecules;
d) hybridising the second nucleic acid molecule to a fourth nucleic acid molecule which comprises a base sequence substantially complementary to the base sequence of the second nucleic acid molecule and which further comprises at least 5 nucleotides which are not complementary to the base sequences of the first or second nucleic acid molecules; wherein the base sequences of the third and fourth nucleic acid molecules are substantially complementary such that under stringent hybridisation conditions said third and fourth nucleic acid molecules hybridise thereby linking said first and second optionally functionalised nanotubes.

8. A method of linking nanotubes comprising the steps of:
a) providing a plurality of optionally functionalised nanotubes with attached nucleic acid molecules, wherein said nucleic acid molecules have the same or different base sequences;
b) exposing said optionally functionalised nanotubes to a nucleotide strand which comprises a base sequence substantially complementary to one or more of the base sequences of said nucleic acid molecules; and
c) incubating said optionally functionalised nanotubes and nucleotide strand under appropriate hybridisation conditions wherein said optionally functionalised nanotubes are linked via hybridisation of the nucleic acid molecules with the nucleotide strand.

9. A plurality of linked nanotubes.

10. Linked nanotubes produced by a method according to claim 5.

11. A method for directing nanotubes to specific targets comprising the steps of:
a) attaching a first nucleic acid molecule of a first base sequence to optionally functionalised nanotubes;
b) attaching a second nucleic acid molecule of a second base sequence which is substantially complementary to the first base sequence to a target; and
c) hybridising said first and second nucleic acid molecules.

12. A method for directing nanotubes to specific targets comprising the steps of:
a) attaching a first nucleic acid molecule of a first base sequence to an optionally functionalised nanotube;
b) attaching a second nucleic acid molecule of a second base sequence to a target;
c) exposing said nanotube and target to a third nucleic acid molecule which comprises a base sequence which is substantially complementary to both the first and second nucleic acid molecules; and
d) incubating said optionally functionalised nanotube and target under appropriate hybridisation conditions wherein said optionally functionalised nanotube and target are linked via hybridisation of the first and second nucleic acid molecule via the third nucleic acid molecule.

13. A method for directing nanotubes to specific targets comprising the steps of:
a) attaching a first nucleic acid molecule of a first base sequence to an optionally functionalised nanotube;
b) attaching a second nucleic acid molecule of a second base sequence to a target;
c) hybridising the first nucleic acid molecule to a third nucleic acid molecule which comprises a base sequence substantially complementary to the base sequence of the first nucleic acid molecule and which further comprises at least 5 nucleotides which are not complementary to the base sequence of the first or second nucleic acid molecules;
d) hybridising the second nucleic acid molecule to a fourth nucleic acid molecule which comprises a base sequence substantially complementary to the base sequence of the second nucleic acid molecule and which further comprises at least 5 nucleotides which are not complementary to the base sequences of the first or second nucleic acid molecules; wherein the base sequences of the third and fourth nucleic acid molecules are substantially complementary such that under stringent hybridisation conditions said third and fourth nucleic acid molecules hybridise thereby directing said optionally functionalised nanotube to said target.

14. A method for directing nanotubes to specific targets comprising the steps of:
a) attaching a first nucleic acid molecule of a first base sequence to optionally functionalised nanotubes;
b) attaching a second nucleic acid molecule of a second base sequence to a target, where the second base sequence is not complementary to the first base sequence, and where the second base sequence may or may not be the same as the first base sequence, and
c) adding a third nucleic acid molecule which has in one part a base sequence substantially complementary to the base sequence of the first nucleic acid molecule and in another part a base sequence substantially complementary to the base sequence of the second nucleic acid molecule; and
d) hybridising the third nucleic acid molecule to the first and the second nucleic acid molecules, thus linking the optionally functionalised nanotube to the target.

15. A method for directing nanotubes to specific targets comprising the steps of:
a) attaching a first nucleic acid molecule of a first base sequence to optionally functionalised nanotubes; and
b) hybridising the first nucleic acid molecule with a second nucleic acid molecule which comprises a base sequence substantially complementary to the first base sequence and further comprises a second or a third base sequence which is/are not complementary to the first base sequence, but is/are complementary to each other.

16. A nucleic acid sensor comprising a nanotube with one or more nucleic acid molecule(s) attached thereto, wherein the base sequence of the said attached nucleic acid molecule is substantially complementary to all or a portion of the base sequence of the nucleic acid molecules being detected.

17. A sensor according to claim 16, wherein the sensor consists of an array of groups of one or more nanotubes, each group having one or more nucleic acid molecules of the same base sequence attached to each nanotube in the group, and where the base sequence of the nucleic acid molecules attached to the nanotubes in one group differs from those in other groups so that a number of different target DNA molecules may be detected.

18. A DNA array consisting of an array of groups of one or more nanotubes, each group having one or more nucleic acid molecules of the same base sequence attached to each nanotube in the group, and where the base sequence of the nucleic acid molecules attached to the nanotubes in one group differs from those in other groups so that a number of different target DNA molecules may be detected.

19. An actuator comprising one or more nanotubes with one or more nucleic acid molecule(s) attached thereto and a membrane support to which the DNA-modified nanotubes are attached.

20. A conductor comprising one or more nanotubes with one or more nucleic acid molecule(s) attached thereto.

21. A conductor according to claim 20, wherein the conductor is a nanowire comprised of nanotubes linked together via nucleic acid hybridisation.

22. A conductor according to claim 21, wherein the nanowire further comprises nanoparticles or coating of conductive material.

23. A method according to claim 2, wherein the nanotubes are carbon nanotubes.

24. A method according to claim 23, wherein carbon nanotubes are grown using a method selected from the group consisting of Arc discharge method, chemical vaporisation deposition method (CVD), plasma enhanced chemical vaporisation deposition method (PECVD), laser ablation/vaporization, pyrolysis, thermal chemical vapour deposition, electrolysis and flame synthesis, or a combination of thereof.

25. A method according to claim 2, wherein the first, second, third or fourth nucleic acid molecules are selected from the group consisting of DNA, cDNA, RNA, oligonucleotide, oligoribonucleotide, modified oligonucleotide, modified oligoribonucleotide and peptide nucleic acid (PNA), or hybrid molecules thereof.

26. A method according to claim 2, wherein the nucleic acid molecule is an oligonucleotide, oligoribonucleotide or RNA-DNA hybrid molecule thereof.

27. A method according to claim 2, wherein the nucleic acid molecule is an oligonucleotide.

28. A method according to claim 2, wherein the nucleic acid molecule is synthesised in a DNA synthesiser or produced by enzymatic digestion or enzymatic polymerisation and then attached onto the optionally functionalised nanotube.

29. A method according to claim 28, wherein the nucleic acid is attached by reacting the nucleic acid molecule with an optionally functionalised nanotube modified with a functional group or with a nanotube physically modified with an anchor containing a functional group.

30. A method according to claim 2, wherein the nucleic acid molecule is synthesised in situ onto a functionalised nanotube or onto a nanotube physically modified with an anchor containing a functional group.

31. A method according to claim 29, wherein the synthesised nucleic acid molecule is attached to a nanotube modified with carboxyl groups either by oxidation or by photo-irradiation of an azido linker containing carboxyl groups.

32. A method according to claim 31, wherein the carboxyl group on the nanotube or on the azido linker forms an amide bond with 5′ or 3′amino modified DNA.

33. A method according to claim 32, wherein the amide bond is extended by incorporating a spacer between the DNA and the linker by using difunctional reagents or non-standard amino acids (C3-C12).

34. A method according to claim 33, wherein the difunctional reagent is one or more amino acids.

35. A method according to claim 34, wherein the non-standard amino acid is 11-amino undecanoic acid.

36. A method according to claim 2, wherein the nucleic acid molecule is synthesised in situ by either oxidizing nanotubes to form hydroxyl groups or attaching functional hydroxyl groups to the nanotubes using photochemical reaction of azido compounds.

37. A method according to claim 36, wherein the azido compound is azido thymidine or azidoadenosine.

38. A method according to claim 2, wherein the DNA is physically attached to the nanotube via a covalent linkage to an anchor which is physically adsorbed to the surface of the nanotube.

39. A method according to claim 38, wherein the DNA is either pre-synthesised or synthesised in situ.

40. A method according to claim 38, wherein the anchor typically contains a hydrophobic domain which interact strongly with the hydrophobic walls of the nanotube, and a functional group to which the DNA can be attached or built-up from.

41. A method according to claim 38, wherein the anchor is an oligonucleotide spacer which physically adsorbs to the nanotube walls and from which extends the hybridizing DNA.

42. A method according to claim 41, wherein the oligonucleotide spacer is oligo thymidine or oligo guanidine.

43. A method according to claim 2, wherein the nucleic acid molecule is attached to the walls(s), side(s) and/or tip(s) of the nanotube.

44. A method according to claim 2, wherein the carbon nanotubes are linked end-to-end, side-to-side, or combinations thereof.

45. A method according to claim 44, wherein the linking process utilises the unique self-annealing properties of nucleic acids.

46. A method according to claim 45, wherein the linking process involves the attachment of a single-stranded nucleic acid molecule to the side or end of a first nanotube, and the attachment of a complementary single-stranded nucleic acid molecule to the side or end of a second nanotube, wherein, under appropriate hybridisation conditions, the nucleic acid molecules hybridise together thereby linking the nanotubes.

47. A method according to claim 46, wherein the linking process involves the attachment of a first single-stranded nucleic acid molecule to the side or end of carbon nanotubes.

48. A linked nanotube according to claim 9 or claim 10, further comprising other nanoparticles.

49. A linked nanotube according to claim 48, wherein the nanoparticles are selected from the group consisting of spheres, rods and octahedrons.

50. A linked nanotube according to claim 49, wherein the nanoparticles are made of gold, silver, and cadmium sulphide (CdS).

51. One or more nanotubes comprising one or more nucleic acid molecule(s) attached thereto further comprising a plurality of nanoparticles hybridized to said nucleic acid molecules by complementary nucleic acid molecules attached to said nanoparticles, wherein said nanotubes are coated with nanoparticles.

52. A method of coating one or more nanotubes with nanoparticles comprising the step of exposing one or more nucleic acid molecule(s) attached to said nanotubes to nanoparticles comprising a plurality of complementary nucleic acid molecules attached thereto, wherein said nanoparticles hybridize to the nucleic acid molecules on the surface of the nanotube(s) as well as self-annealling to other nanoparticles thereby forming one or more coated nanotubes.
